SEQUENCE LISTING (49 sequences)

SEQ ID NO: 1 - protein, 23 aa, Influenza virus
SLLTEVETPIRNEWGCRCNDSSD

SEQ ID NO: 2 - protein, 23 aa, Influenza virus
SLLTEVETPIRNEWECRCNGSSD

SEQ ID NO: 3 - protein, 22 aa, Influenza virus
SLLTEVETPIRNEGCRCNDSSD

SEQ ID NO: 4 - protein, 23 aa, Influenza A virus
SLLTEVETPIRNEWGCRCNGSSD

SEQ ID NO: 5 - protein, 23 aa, Influenza A virus
SLLTEVETPTKNEWECRCNDSSD

SEQ ID NO: 6 - protein, 333 aa, Influenza A virus
QNLPGNDNSTATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEGFNWTGVTQNGGSYACKRGPDNGFFSRLNWLYKSESTYPVLNVTMPNNGNFDKLYIWGVHHPSTDKEQTNLYVQASGRVTVSTKRSQQTIIPNVGSRPWVRGLSSRISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGLFG

SEQ ID NO: 7 - protein, 18 aa, Influenza A virus
SKAFSNCYPYDVPDYASL

SEQ ID NO: 8 - protein, 498 aa, Influenza A virus
MASQGTKRSYEQMETDGERQNATEIRASVGKMIDGIGRFYIQMCTELKLSDYEGRLIQNSLTIERMVLSAFDERRNKYLEEHPSAGKDPKKTGGPIYKRVDGKWMRELVLYDKEEIRRIWRQANNGDDATRGLTHMMIWHSNLNDTTYQRTRALVRTGMDPRMCSLMQGSTLPRRSGAAGAAVKGIGTMVMELIRMIKRGINDRNFWRGENGRKTRSAYERMCNILKGKFQTAAQRAMMDQVRESRNPGNAEIEDLIFSARSALILRGSVAHKSCLPACVYGPAVASGYDFEKEGYSLVGIDPFKLLQNSQVYSLIRPNENPAHKSQLVWMACHSAAFEDLRLLSFIRGTKVSPRGKLSTRGVQIASNENMDTMESSTLELRSRYWAIRTRSGGNTNQQRASAGQISVQPTFSVQRNLPFDKSTIMAAFTGNTEGRTSDMRAEIIRMMEGAKPEEVSFRGRGVFELSDEKATNPIVPSFDMSNEGSYFFGDNAEEYDN

SEQ ID NO: 9 - protein, 15 aa, Influenza A virus
RLIQNSLTIERMVLS

SEQ ID NO: 10 - protein, 12 aa, Influenza A virus
TYQRTRALVRTG

SEQ ID NO: 11 - protein, 190 aa, Cowpea Chlorotic Mottle Virus (CCMV)
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 12 - protein, 213 aa, Cowpea Mosaic Virus (CPMV)
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPLLKFRFRDIERSKRSVMVGHTATAA

SEQ ID NO: 13 - protein, 374 aa, Cowpea Chlorotic Mottle Virus (CCMV)
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 14 - DNA, 53 bp, Synthetic Primer
cggggatcctgtcactcttgacagaggtagaaacaccgatacgtaatgaatgg

SEQ ID NO: 15 - protein, 568 aa, Influenza A virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 16 - DNA, 1614 bp, Influenza A virus
atggagaagatagttctcttgtttgccatcgtcagtttggtcaaatcagatcagatttgtataggataccatgcaaacaacagtaccgaacaagttgacacaatcatggagaagaatgtaacagtgactcacgcccaggacattcttgagaagacccacaatggcaagctttgcgacttggatggtgttaagccactcattcttcgtgattgttctgtggcaggttggcttctcggaaacccaatgtgtgacgagttcatcaacgttccagagtggtcttacatcgtcgagaaggcaaaccctgtgaatgatctttgctacccaggagacttcaacgactacgaggaattgaaacatctcttgtctaggatcaaccactttgagaagattcagatcattcctaagtcctcttggtcttcacatgaggcaagccttggtgtgtcatccgcctgcccttatcaaggaaagtcatctttcttcagaaatgttgtgtggcttatcaagaagaactctacatatccaaccatcaagaggagctacaacaacacaaaccaggaagatctcttggtgctctggggaattcatcatccaaatgacgcagcagagcaaactaagctttaccagaaccctacaacttacatctccgtgggcacttctacactcaatcagagacttgtgccaaggattgctactaggtcaaaggttaacggacaatcaggtcgtatggagttcttctggacaatcttgaagccaaacgatgccatcaacttcgagtcaaatggaaacttcatcgctccagagtacgcttacaagattgtgaagaaaggagatagtaccatcatgaagtctgaactcgagtacggaaactgcaacaccaagtgtcagactccaatgggagctatcaatagctctatgccatttcacaacattcaccctttgacaataggagaatgccctaagtacgtgaagagcaacaggctcgtcctcgcaactggtttgagaaacagtccacaaagagaacgtagacgtaagaagagaggattgttcggtgcaattgccgggttcatcgaaggaggctggcagggtatggtggatggttggtatgggtatcatcacagtaatgagcaaggatcaggatatgctgcagacaaagaaagcacccagaaagcaatagatggagtcactaacaaagtcaattccataatcgacaagatgaacacacagttcgaagctgttggacgtgagttcaacaaccttgagaggaggattgagaatcttaacaagaagatggaagatgggttcttggacgtgtggacttacaatgctgaattgttagttcttatggagaacgaaagaactctcgacttccatgattctaacgtgaagaacttgtacgacaaggtgcgtcttcaacttcgtgataacgctaaagagctcgggaacggttgctttgagttctatcacaagtgtgacaatgagtgcatggaatctgttagaaatggaacttacgattaccctcagtattcagaggaggcaaggctcaagagagaagagatctccggcgtgaagttggagagcattggtatctaccaacatcatcaccatcaccactaa

SEQ ID NO: 17 - protein, 537 aa, Influenza A virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQHHHHHH

SEQ ID NO: 18 - protein, 352 aa, Influenza A virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRHHHHHH

SEQ ID NO: 19 - DNA, 1059 bp, Influenza A virus
atggagaagatagttctcttgtttgccatcgtcagtttggtcaaatcagatcagatttgtataggataccatgcaaacaacagtaccgaacaagttgacacaatcatggagaagaatgtaacagtgactcacgcccaggacattcttgagaagacccacaatggcaagctttgcgacttggatggtgttaagccactcattcttcgtgattgttctgtggcaggttggcttctcggaaacccaatgtgtgacgagttcatcaacgttccagagtggtcttacatcgtcgagaaggcaaaccctgtgaatgatctttgctacccaggagacttcaacgactacgaggaattgaaacatctcttgtctaggatcaaccactttgagaagattcagatcattcctaagtcctcttggtcttcacatgaggcaagccttggtgtgtcatccgcctgcccttatcaaggaaagtcatctttcttcagaaatgttgtgtggcttatcaagaagaactctacatatccaaccatcaagaggagctacaacaacacaaaccaggaagatctcttggtgctctggggaattcatcatccaaatgacgcagcagagcaaactaagctttaccagaaccctacaacttacatctccgtgggcacttctacactcaatcagagacttgtgccaaggattgctactaggtcaaaggttaacggacaatcaggtcgtatggagttcttctggacaatcttgaagccaaacgatgccatcaacttcgagtcaaatggaaacttcatcgctccagagtacgcttacaagattgtgaagaaaggagatagtaccatcatgaagtctgaactcgagtacggaaactgcaacaccaagtgtcagactccaatgggagctatcaatagctctatgccatttcacaacattcaccctttgacaataggagaatgccctaagtacgtgaagagcaacaggctcgtcctcgcaactggtttgagaaacagtccacaaagagaacgtagacgtaagaagagacatcatcaccatcaccactaa

SEQ ID NO: 20 - protein, 207 aa, Influenza A virus
MEKIVLLFAIVSLVKSGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQHHHHHH

SEQ ID NO: 21 - DNA, 624 bp, Influenza A virus
atggagaagatagttctcttgtttgccatcgtcagtttggtcaaatcaggattgttcggtgcaattgccgggttcatcgaaggaggctggcagggtatggtggatggttggtatgggtatcatcacagtaatgagcaaggatcaggatatgctgcagacaaagaaagcacccagaaagcaatagatggagtcactaacaaagtcaattccataatcgacaagatgaacacacagttcgaagctgttggacgtgagttcaacaaccttgagaggaggattgagaatcttaacaagaagatggaagatgggttcttggacgtgtggacttacaatgctgaattgttagttcttatggagaacgaaagaactctcgacttccatgattctaacgtgaagaacttgtacgacaaggtgcgtcttcaacttcgtgataacgctaaagagctcgggaacggttgctttgagttctatcacaagtgtgacaatgagtgcatggaatctgttagaaatggaacttacgattaccctcagtattcagaggaggcaaggctcaagagagaagagatctccggcgtgaagttggagagcattggtatctaccaacatcatcaccatcaccactaa

SEQ ID NO: 22 - protein, 22 aa, Influenza virus
SLLTEVETPIRNEECRCNGSSD

SEQ ID NO: 23 - protein, 22 aa, Influenza A virus
SLLTEVETPIRNEGCRCNGSSD

SEQ ID NO: 24 - protein, 22 aa, Influenza A virus
SLLTEVETPTKNEECRCNDSSD

SEQ ID NO: 25 - protein, 565 aa, Influenza A virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFDDYEELKHLLSRINHFEKIQIIPKSSWSSHEASLGVSSACPYQGKSSFFRNVVWLIKKNSTYPTIKRSYNNTNQEDLLVMWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCR

SEQ ID NO: 26 - DNA, 1732 bp, Influenza A virus
ggactagtaggaggtaacttatggagaaaatcgtcctgttgtttgccattgtctccctggtgaagagcgaccagatttgcatcggctatcacgcgaacaattccaccgaacaagtggatacgatcatggagaagaatgtgaccgtcacccacgctcaggatattctggagaagacgcataacgggaaactctgtgacttggatggggttaagccgctgattctgcgcgattgttcggtggccggctggctgctgggcaacccaatgtgcgatgaatttatcaacgtgcccgagtggagctacattgtcgagaaggccaatcccgttaacgacttgtgctaccctggtgatttcgacgactacgaagaactgaagcacctgttgtcccgcattaatcacttcgagaaaatccagatcatcccgaaatcgagctggagcagccatgaagcctcgctcggtgtgagttccgcctgtccgtaccagggcaagtcgtccttcttccgtaacgtggtgtggctgattaagaagaactccacttacccgaccattaagcggagctacaacaacaccaaccaagaagacttgttggtgatgtggggtatccatcaccccaacgacgccgccgagcaaaccaaactgtaccagaatcctacgacttacatctcggtcggcaccagcaccctgaaccaacgcttggttccgcgcatcgcgactcgcagcaaagtcaacggccagagtgggcgtatggaattcttttggaccatcctgaagccaaacgatgcgatcaacttcgaatcgaatggcaacttcattgccccggaatacgcctacaagatcgtgaagaaaggggactcgaccatcatgaagtcggagctggaatacggcaactgcaacacgaaatgccagacgccgatgggcgccatcaactccagcatgccgtttcataacattcacccattgactatcggcgaatgcccgaaatacgtcaagtccaatcgtctggtcctggcgaccggtctgcgcaacagcccgcagcgcgaacgtcgccgtaagaaacggggcctgttcggtgccatcgctggcttcatcgagggcggctggcagggcatggtcgacggctggtatggctaccatcacagcaacgagcagggcagtggttacgccgctgacaaggaaagcacccaaaaggccatcgacggcgtgacgaacaaggtgaactccattatcgacaagatgaacacgcagttcgaagccgtcggccgtgagttcaacaacctggaacgccgcatcgaaaacttgaacaagaagatggaagacggtttcttggacgtctggacctataatgcggaattgctggttctgatggaaaacgaacgcaccctggactttcatgactcgaacgtgaagaacctgtatgataaagtccgtctgcagctgcgcgacaacgccaaggaactgggtaacggctgctttgaattttaccataaatgtgacaatgagtgcatggaaagtgtgcgcaacggcacctatgattatccgcagtacagtgaagaggcacgtctgaagcgtgaggaaattagcggcgttaaattggagagcatcgggatctatcagatcctcagcatctacagcaccgtggccagcagcttggccctggccatcatggtcgctggcctctcgctgtggatgtgcagcaacggttccctgcagtgccgctgataatagctcgagtt

SEQ ID NO: 27 - DNA, 1732 bp, Influenza A virus
ggactagtaggaggtaacttatggaaaagattgtgctgttgttcgccatcgtgagtctggtgaaatcggaccaaatctgcatcggctaccacgctaataacagcaccgaacaagtcgacaccatcatggagaagaacgtcactgtgacgcatgcccaagatatcttggaaaagacccataacggcaagctgtgcgacctggacggtgtgaagccgttgatcctgcgcgactgctccgtcgcgggttggctgttgggcaacccgatgtgcgatgagttcattaacgtcccggaatggagctatatcgtcgagaaggcgaatcccgtcaacgacctgtgttaccctggcgatttcgatgattacgaagagctgaaacatctgctgagccgcatcaaccacttcgagaagatccaaatcatcccgaagagcagttggagcagccacgaagcctccctgggcgtttcgtcggcctgcccctatcaggggaagtcgtcctttttccgcaacgtggtctggctgatcaaaaagaacagtacctatcctactatcaagcgcagttacaacaacactaaccaagaagacctgttggtcatgtggggcattcatcatcccaacgacgcggccgagcagaccaagttgtaccagaacccgaccacgtatatcagcgtggggacgtccaccctcaatcagcgtctggtgccgcgcatcgcgacccgtagcaaggtgaacgggcagtcgggccggatggagttcttttggactatcctgaagccgaacgacgcaatcaacttcgagtcgaatggtaacttcattgccccagagtatgcttacaagatcgtgaaaaagggcgactcgactatcatgaagagcgaactggagtacgggaactgtaacaccaaatgtcaaaccccgatgggcgcaatcaacagctcgatgcccttccataatatccatccgctgaccattggtgagtgcccgaagtacgtcaaatcgaaccggttggtgctggccactggcctccgtaactcgccgcagcgggaacgtcgccgtaagaaacgcggtttgttcggcgccattgcagggttcatcgagggcggctggcagggcatggtcgatggttggtacgggtaccaccactccaacgaacaaggcagcggctacgcggcggataaagaaagtacccagaaggctatcgacggcgtcaccaacaaagtgaacagcatcatcgataagatgaacacgcagttcgaagccgtgggccgtgagttcaacaacctcgaacggcgcatcgagaacctgaacaaaaagatggaagatggcttcctggatgtctggacctataatgccgagctgctggtgctgatggaaaacgagcgtaccctggactttcacgattcgaatgtgaagaatctgtacgacaaagtccggttgcagctgcgcgacaacgcgaaagagctgggcaacggctgtttcgagttctaccataagtgcgacaacgagtgtatggagtccgtgcgcaacggcacgtatgattatcctcagtattccgaagaggcccgcttgaaacgtgaagaaatcagcggcgtgaagctggagagcatcggcatctatcaaatcttgagcatctatagcaccgtggcgtcgtcgctggccctcgcgatcatggttgccggcctgagcctgtggatgtgcagcaacggctcgctgcaatgccgctgataatagctcgagtt

SEQ ID NO: 28 - DNA, 1732 bp, Influenza A virus; identical to SEQ ID NO: 26

SEQ ID NO: 29 - protein, 515 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPVNDLCYPGDFDDYEELKHLLSRINHFEKIQIIPKSSWSSHEASLGVSSACPYQGKSSFFRNVVWLIKKNSTYPTIKRSYNNTNQEDLLVMWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQ

SEQ ID NO: 30 - DNA, 54 bp, Synthetic Primer
cgcaggatcccatctgaagaatcattacaacgacagccccattcattacgtatc

SEQ ID NO: 31 - DNA, 53 bp, Synthetic Primer; identical to SEQ ID NO: 14

SEQ ID NO: 32 - DNA, 50 bp, Synthetic Primer
cgcaggatcccatctgaagagccattacaacgacattcccattcattacg

SEQ ID NO: 33 - DNA, 54 bp, Synthetic Primer
gatcctgcgcctgatccagaacagcctgaccatcgaacgcatggtgctgagcgg

SEQ ID NO: 34 - DNA, 54 bp, Synthetic Primer
gatcccgctcagcaccatgcgttcgatggtcaggctgttctggatcaggcgcag

SEQ ID NO: 35 - DNA, 45 bp, Synthetic Primer
gatcctgacctaccagcgcacccgcgctctggtgcgcaccggcgg

SEQ ID NO: 36 - DNA, 45 bp, Synthetic Primer
gatcccgccggtgcgcaccagagcgcgggtgcgctggtaggtcag

SEQ ID NO: 37 - DNA, 63 bp, Synthetic Primer
gatcctgagcaaggctttcagcaactgctacccgtacgacgtgccggactacgctagcctggg

SEQ ID NO: 38 - DNA, 63 bp, Synthetic Primer
gatccccaggctagcgtagtccggcacgtcgtacgggtagcagttgctgaaagccttgctcag

SEQ ID NO: 39 - DNA, 69 bp, Synthetic Primer
atggatagctagcactcctcctgctagtctgctgaccgaagtggaaaccccgattcgcaacgaaggctg

SEQ ID NO: 40 - DNA, 64 bp, Synthetic Primer
tgcctgtgacgtctgaaaatggatcgctgctatcgttgcagcggcagccttcgttgcgaatcgg

SEQ ID NO: 41 - DNA, 30 bp, Synthetic Primer
gcgcgatatcaacaatggagaagatagttc

SEQ ID NO: 42 - DNA, 46 bp, Synthetic Primer
gcgctccggatttagtggtgatggtgatgatgtctcttcttacgtc

SEQ ID NO: 43 - DNA, 70 bp, Synthetic Primer
gcgatatcaacaatggagaagatagttctcttgtttgccatcgtcagtttggtcaaatcaggattgttcg

SEQ ID NO: 44 - DNA, 44 bp, Synthetic Primer
gcgctccggatttagtggtgatggtgatgatgttggtagatacc

SEQ ID NO: 45 - DNA, 41 bp, Synthetic Primer
ggactagtaggaggtaacttatgaaactgaaacgtttgatg

SEQ ID NO: 46 - DNA, 39 bp, Synthetic Primer
gtgatagccgatgcaaatctggtcggccaccgcgttggc

SEQ ID NO: 47 - DNA, 39 bp, Synthetic Primer
gccaacgcggtggccgaccagatttgcatcggctatcac

SEQ ID NO: 48 - DNA, 37 bp, Synthetic Primer
ccgctcgagtcattactgatagatcccgatgctctcc

SEQ ID NO: 49 - DNA, 41 bp, Synthetic Primer; identical to SEQ ID NO: 45

(19) United States
(12) Patent Application Publication
(10) Pub. No.: US 2009/0117144 A1
(43) Pub. Date: May 7, 2009

(54) Recombinant flu vaccines

(76) Inventors: Lada Rasochova, Del Mar, CA (US); Nghiep Dang, Ventura, CA (US); Philip P. Dao, San Diego, CA (US); Jamie P. Phelps, Aurora, CO (US); Jason Radam, San Diego, CA (US)

Correspondence Address: TRASKBRITT, P.C.\Dow Global Technologies Inc., PO Box 2550, SALT LAKE CITY, UT, 84110, US

(21) Appl. No.: 11/489,691

(22) Filed: Jul. 19, 2006

Related U.S. Application Data
(60) Provisional application No. 60/700,601, filed on Jul. 19, 2005.

(51) Int. Cl. A61K 39/145; C07K 14/00; A61P 37/04

(52) U.S. Cl. 424/192.1; 530/326

(57) ABSTRACT

The present invention provides compositions for use as vaccines against the influenza virus, and rapid methods of producing such compositions. The composition include i) at least one peptide derived from an influenza virus, wherein the peptide is fused to a capsid protein derived from a plant virus forming a recombinant capsid fusion peptide and ii) at least one isolated antigenic protein or protein fragment derived from a human or avian influenza virus. The isolated antigenic protein or protein fragment derived from the human or avian influenza virus can be conjugated to the surface of the recombinant capsid fusion peptide.

### CROSS REFERENCE TO RELATED APPLICATIONS

This application claims priority to U.S. Provisional Application No. 60/700,601, filed Jul. 19, 2005.

### FIELD OF THE INVENTION

The present invention is directed to the production and assembly of multivalent influenza virus vaccines utilizing isolated influenza antigenic proteins or protein fragments derived from human and/or avian influenza viruses combined with an adjuvant comprising a chimeric virus like particle carrier containing a viral capsid protein derived from a eukaryotic or prokaryotic cell genetically fused to human and/or avian influenza virus antigenic peptides. The present invention is also directed to novel antigenic peptides, and compositions containing such peptides, derived from influenza proteins.

### BACKGROUND OF THE INVENTION

A course of vaccinations is one of the most effective and efficient ways to protect animals and humans from infections by pathogenic agents. In general, vaccines are designed to provide protective immunity from a pathogenic agent by eliciting a host immune response to the antigenic proteins, peptides or other immunogenic structures contained in the vaccine, thus reducing the potential for successful infection upon exposure of the host to the pathogenic agent.

The influenza virus, is a member of the Orthomyxoviridae family, and includes three subtypes classified by their core proteins, designated influenza A, influenza B, and influenza C. Influenza A viruses infect a range of mammalian and avian species, whereas types B and C are essentially restricted to human infection. Influenza A viruses are generally responsible for annual epidemics and occasional pandemics, whereas influenza B viruses cause outbreaks every 2-4 years, but are not generally associated with pandemics. Virus strains are classified according to host species of origin, geographic site, year of isolation, serial number, and, for influenza A, by serological properties of subtypes of haemagglutinin and neuraminidase.

The influenza virus is a segmented negative-sense RNA virus essentially composed of nine proteins: matrix (M1); proton-ion channel (M2); hemagglutinin (HA), neuraminidase (NA); nucleoprotein (NP); polymerase basic protein 1 (PB1); polymerase basic protein 2 (PB2); polymerase acidic protein (PA); and nonstructural protein 2 (NP2). The HA, NA, M1, and M2 proteins are membrane associated proteins, with the HA and NA proteins being glycoproteins responsible for viral attachment and entry into the host cell, respectively. Fifteen classes of hemagglutinin antigens, classified H1-H15, and 9 classes of neuraminidase antigens, classified N1-N9, have been identified in influenza A viruses.

The HA protein initializes viral attachment to the cell by binding to a host cell surface receptor that contains sialic acid. The hemagglutinin of human influenza viruses preferentially binds to sialic acid receptors containing α2,6-galactose linkages, whereas avian influenza viruses preferentially bind to cells containing α2,3-galactose linkages. These binding preferences correlate with the predominance of sialic acid α2,6-galactose linkages on human epithelial cells, and α2,3-galactose linkages on avian intestinal epithelial cells. See, for example, Rogers G N, Paulson J C, Daniels R S, Skehel J J, Wilson I A, Wiley D C (1983) “Single amino acid substitutions in influenza haemagglutinin change receptor binding specificity,” Nature 304: 76-78; Connor R J, Kawaoka Y, Webster R G, Paulson J C (1994) “Receptor specificity in human, avian and equine H2 and H3 influenza virus isolates,” Virology 205: 17-23; Ito T, Suzuki Y, Mitnaul L, Vines A, Kida H, Kawaoka Y (1997) “Receptor specificity of influenza A viruses correlate with agglutination of erythrocytes from different animal species,” Virology 227:492-99. Although the molecular mechanisms responsible for receptor-binding specificity are poorly defined, it is believed that influenza hemagglutinin of avian origin must acquire human receptor-binding specificity to generate influenza strains capable of sustained human-to-human transmission. See, for example, Stephenson I, K G Nicholson, J M Wood, M C Zambon, and J M Katz (2004) “Confronting the avian influenza threat: vaccine development for a potential pandemic,” The Lancet Infectious Diseases 4:499-509. Site-directed mutagenesis studies have shown that only one or two amino acid mutations are required for this change. See Matrosovich M, Tuzikov A, Bovin N, et al. (2000) “Early alterations of the receptor binding properties of the H1, H2 and H3 avian influenza virus hemagglutinins after their introduction into mammals,” J Virol 74: 8502-12.

Once attachment occurs, the NA protein initiates receptor mediated endocytosis, and host cell/viral membrane fusion. The HA protein then undergoes a conformational change in the acidic environment of the endosome, and, along with the M2 protein, mediates the release of M1 proteins from nucleocapsid-associated ribonucleoproteins (RNPs), which are then directed to the cell nucleus for viral RNA synthesis.

The M2 protein is a 97 amino acid non-glycosylated transmembrane protein. Lamb R A, Lai C-J, Choppin P W (1981) “Sequences of mRNAs derived from genome RNA segment 7 of influenza virus: collinear and interrupted mRNAs code for overlapping proteins,” PNAS 78:4170-4; Lamb R A, Zebedee S L, Richardson C D (1985) “Influenza virus M2 protein is an integral membrane protein expressed on the infected-cell surface,” Cell 40:627-33. It forms homotetramers in the viral membrane of the virus particle, but at comparatively low numbers when compared to HA and NA. However, they are present in high density in the plasma membrane of the infected cell. Zebedee S L, Lamb R A (1988) “Influenza A virus M2 protein: monoclonal antibody restriction of virus growth and detection of M2 in virions,” J Virol 62:2762-72.

The M2 protein is believed to facilitate the release of RNP complexes from the viral membrane after fusion. It exhibits proton transport activity that reduces the pH within transport vesicles during egress of viral transmembrane proteins from the ER to the plasma membrane, preventing a premature acid induced conformational change in HA. See Mozdzanowska K et al (2003) “Induction of influenza type A virus specific resistance by immunization of mice with a synthetic multiple antigenic peptide vaccine that contains ectodomains of matrix protein 2,” Vaccine 21:2616-2626; Steinhauer D A, Wharton S A, Skehel J J, Wiley D C, Hay A J (1991) “Amantadine selection of a mutant influenza virus containing an acid-stable hemagglutinin glycoprotein: evidence for virus-specific regulation of the pH of glycoprotein transport vesicles,” Proc Natl Acad Sci 88:11525-9; Pinto L H, Holsinger L J, Lamb R A (1992) “Influenza virus M2 protein has ion channel activity,” Cell 69:517-28; Zhimov O P (1990) “Solubilization of matrix protein M1/M from virions occurs at different pH for orthomyxo- and paramyxoviruses,” Virology 176:274-9.

The M2 protein contains a 23 amino-acid long ectodomain (M2e) that is highly conserved amongst influenza type A viruses capable of infecting humans. In fact, the 9 N-terminal amino acids are totally conserved across the infectious human strains of the virus, and there is only a minor degree of structural diversity is shown in the first 15 N-terminal amino acids. Zebedee S L, Lamb R A (1988) “Influenza A virus M2 protein: monoclonal antibody restriction of virus growth and detection of M2 in virions,” J Virol 62:2762-72; Ito T, Gorman O T, Kawaoka Y, Bean W J, Webster R G (1991) “Evolutionary analysis of the influenza A virus M gene with comparison of the M1 and M2 proteins,” J. Virol. 65:5481-8.

Generally, avian influenza viruses are incapable of efficient replication in humans. Beare A S, Webster R G (1991) “Replication of avian influenza viruses in humans,” Arch Virol 119: 37-42. However, it is known that some subtypes of avian influenza viruses can replicate within the human respiratory tract. There have been a number of confirmed cases of transmission of avian influenza virus to humans. See Stephenson I, K G Nicholson, J M Wood, M C Zambon, and J M Katz (2004) “Confronting the avian influenza threat: vaccine development for a potential pandemic,” The Lancet Infectious Diseases 4:499-509; WHO disease alert (2004) “Confirmed human cases of avian influenza H5N1,” http://www.who.int/csr/disease/avian_influenza/en/; Hien T T, Liem N T, Dung N T, et al (2004) “Avian influenza (H5N1) in 10 patients in Vietnam,” N Engl J Med 350: 1179-88. The ability of certain types of avian influenza viruses to infect humans increases the pool of species that can provide an environment for avian/human reassortant virus emergence.

In general, two types of influenza vaccines exist, the inactivated whole influenza viral vaccine and the inactivated subvirion viral vaccine. The whole viral vaccine contains intact, inactivated virions, while the subvirion vaccine contains most of the viral structural proteins and some of the viral envelope proteins. These viral vaccines are composed annually of a trivalent blend of influenza type A and influenza type B strains predicted to be in circulation among the human population for a given flu season. The WHO reviews vaccine composition biannually and updates antigenic content depending on prevalent circulating subtypes to provide antigenically well-matched vaccines. For example, for the 2004-2005 flu season, the trivalent composition comprised the A/New Caledonia/20/99 (H1N1); A/Wyoming/03/2003 (H3N2), which is an A/Fujian/411/2002-like virus; and B/Shanghai/361/2002-like virus (i.e. B/Jiangsu/10/2003 or B/Jilin/20/2003). Examples of such vaccines include Fluzone (Connaught), Fluvirin (Chiron), and Flu-Shield (Wyeth-Lederle). Recently, MedImmune has developed a live attenuated influenza vaccine for intranasal delivery, FluMist, which has received approval from the FDA for commercial usage in the United States. These vaccines generally produce a strain-specific humoral response, have reduced efficacy against antigenically drifted viruses, and are ineffective against unrelated strains. See Stephenson I, Nicholson K G, Wood J M, Zambon M C, and Katz J M (2004) “Confronting the avian influenza threat: vaccine development for a potential pandemic,” The Lancet: Infectious Diseases 4:499-509.

The inactivated and attenuated viruses utilized in the above described vaccinations are produced in the allantoic cavity of embryonated chick eggs. This production method is time consuming, taking up to 6 months to produce and can be highly vulnerable to contamination. In 2004, contamination in the production of the influenza virus by Chiron resulted in a highly publicized and controversial shortage of flu vaccine. The contamination was discovered in August of 2004, too late for the manufacturers to generate new batches of vaccine for that season. In addition, the current production methods require anticipating the particular strain or strains that are most likely to emerge during the flu season. Such a requirement, in conjunction with the current production methods, limit the ability to modify production of an influenza vaccine to target an unexpected viral strain.

Thus, there is a need for improved vaccines that can be rapidly produced and can be easily modified to allow vaccination against newly emerging viruses.

### SUMMARY OF THE INVENTION

The present invention provides compositions for use as vaccines against a virus, particularly an influenza virus comprising i) at least one peptide derived from an influenza virus fused to at least one capsid protein derived from a plant virus forming a recombinant capsid fusion peptide, wherein the recombinant capsid fusion peptide is capable of assembly to form a virus or virus like particle, and ii) at least one isolated antigenic protein or protein fragment derived from a human or avian influenza virus. Such a strategy utilizes the immunogenic aspect of a virus or virus like particle in combination with antigenic proteins or protein fragments to produce a vaccine that may provide broader protective immunity against human and/or avian influenza viruses.

In one aspect of the present invention, the peptide derived from an influenza virus fused to the plant capsid protein is a conserved influenza viral epitope. In one embodiment, the conserved epitope is a conserved human influenza virus epitope. By utilizing a conserved influenza epitope as an antigenic insert for the virus or virus like particle, the core component of the composition need not be re-engineered on a yearly basis. Instead, only the isolated antigenic protein or protein fragment need change as new strains of influenza virus emerge. Because the antigenic proteins or protein fragments can be recombinantly produced, the composition can be rapidly produced for use as a vaccine to elicit an immune response in a human or animal against newly emergent influenza strains.

In a specific embodiment, the conserved influenza peptide is derived from the M2 protein. In one embodiment, the M2 derived peptide is selected from the group consisting of SEQ ID Nos: 1-5, and 22-24. Embodiments of the present invention provide M2 influenza protein derived peptide sequences selected from the group consisting of SEQ ID Nos: 3, 22, 23, and 24. Additionally, fragments, derivatives and homologs of SEQ ID No: Nos. 3, 22, 23, or 24 are provided. In other embodiments, the conserved epitope is derived from the NP protein. In one embodiment, the NP peptide is selected from the group consisting of SEQ ID Nos: 8-10. In another embodiment, the conserved epitope is derived from the HA protein. In one embodiment, the HA peptide is selected from the group consisting of SEQ ID Nos: 6 and 7.

In other embodiments, any combination of conserved influenza peptides derived from an influenza virus selected from the group consisting of M2, NP, or HA can be fused to a capsid protein. In one embodiment, the capsid fusion peptide contains an M2, NP, and HA peptide. In another embodiment, the capsid fusion peptide contains an M2 and an NP conserved peptide. In still another embodiment, the capsid fusion peptide contains an M2 and an HA peptide. In another embodiment, the capsid fusion peptide contains an HA and an NP conserved peptide.

The present invention utilizes capsid proteins derived from plant viruses to construct capsid fusion peptides. The capsid proteins with the fused influenza peptide can self-assemble in vivo or in vitro to form a virus or virus like particle. In one embodiment, the virus or virus like particle does not include host cell plasma membrane proteins or host cell wall proteins. In one embodiment, the plant virus will be selected from viruses that are icosahedral (including icosahedral proper, isometric, quasi-isometric, and geminate or “twinned”), polyhedral (including spherical, ovoid, and lemon-shaped), bacilliform (including rhabdo- or bullet-shaped, and fusiform or cigar-shaped), and helical (including rod, cylindrical, and filamentous). In some embodiments the plant virus can be an icosahedral plant virus species. In one embodiment, the viral capsid can be derived from a Cowpea Chlorotic Mottle Virus (CCMV) or a Cowpea Mosaic Virus (CPMV). In additional embodiments the plant virus is selected from a CCMV or CPMV virus, and the capsid includes at least one insert selected from the group consisting of SEQ ID Nos: 3, 22, 23, and 24.

In one aspect of the present invention, the isolated antigenic protein or protein fragment combined with the virus or virus like particle is an influenza protein from a newly emergent influenza viral strain, including a human or avian influenza virus. In one embodiment, the protein or protein fragment is derived from an avian influenza virus. In one embodiment of the present invention, the antigenic protein or protein fragment is derived from an influenza viral protein selected from the group consisting of matrix (M1), proton-ion channel (M2), hemagglutinin (HA), neuraminidase (NA), nucleoprotein (NP), polymerase basic protein 1 (PB1), polymerase basic protein 2 (PB2), polymerase acidic protein (PA), and nonstructural protein 2 (NP2). In one embodiment of the present invention, the protein or protein fragment is derived from an avian influenza HA or NA.

In certain embodiments, the virus or virus like particle is combined with more than one isolated antigenic protein or protein fragment. In certain embodiments, these isolated antigenic peptide or peptide fragments are derived from the same species. In other embodiments, these isolated antigenic peptide or peptide fragments are derived from different species. In certain embodiments, the virus or virus like particle Is combined with at least one NA protein or protein fragment and at least one HA protein or protein fragment. In certain embodiments, the NA and/or the HA fragments are derived from an avian influenza virus. In certain other embodiments, the NA and/or the HA fragments are derived from a human influenza virus. In an additional embodiment, the virus like particle is combined with at least one NA protein or protein fragment, at least one HA protein or protein fragment, and any combination of avian influenza viral proteins or protein fragments selected from the group consisting of M1, M2, NP, PB1, PB2, PA, and NP2. In certain embodiments the NA protein or protein fragment is derived from the group of influenza NA proteins selected from the group consisting of N1, N2, N3, N4, N5, N6, N7, N8, and N9. In additional embodiments the HA protein or protein fragment is derived from influenza H1, H2, H3, H4, H5, H6, H7, H8, H9, H10, H11, H12, H13, H14, and H15

In certain embodiments, the isolated antigenic peptide is in a mixture with the virus or virus like particle but is not covalently linked to the virus or virus like particle. The mixture can include additional excipients. In one embodiment, at least one antigenic protein fragment is less than the full length protein. In certain embodiments, the antigenic protein fragment is derived from an avian or human influenza virus. In certain embodiments, the protein fragment comprises at least 10, 15, 20, 25, 50, 75, 100, 150, 200 or more amino acids.

In one embodiment, the peptide(s) derived from an influenza virus, the capsid protein(s) derived from a plant virus, and the antigenic protein(s) or protein fragment(s) derived from an influenza virus can be altered to provide for increased desirable characteristics. Such characteristics include increased antigenicity, increased recombinant expression in a host cell, more efficient assembly, or improved covalent binding properties. In one embodiment, the influenza peptide inserted into the capsid protein is modified by changing its amino acid sequence, wherein the alteration does not reduce the antigenic nature of the peptide. In another embodiment, the influenza peptide inserted into the capsid protein is modified by post-translational modifications, such as glycosylation, phosphorylation or lipid modification. In another embodiment, the isolated antigenic protein or protein derived fragment can be modified by changing its amino acid sequence, wherein the alteration does not reduce the antigenic nature of the peptide. In another embodiment, the isolated antigenic protein or protein derived fragment is modified by post-translational modification.

In other embodiments of the present invention, at least one isolated protein or protein fragment can be covalently attached to the surface of the peptide-containing virus or virus like particle. In another embodiment, at least one avian or human influenza viral protein fragment consisting of less than the entire amino acid sequence of the protein is covalently attached to the surface of the peptide containing virus or viral like particle. In one embodiment, the covalently linked antigenic protein fragment includes at least 10, 15, 20, 25, 50, 75, 100, 150, 200 or more amino acids.

In another embodiment of the present invention, at least one M2, NP, or HA peptide derived from an influenza virus is fused to a capsid protein derived from a plant virus forming a first recombinant capsid fusion peptide and the recombinant capsid fusion peptide is combined with at least one peptide derived from an avian and/or human influenza virus fused to a capsid protein derived from a plant virus forming a second recombinant capsid fusion peptide. In this embodiment, the first recombinant capsid fusion peptide and second recombinant capsid fusion peptide are capable of assembly, in vivo or in vitro, to form a virus or virus like particle. The resultant virus like particle can then be combined with an isolated antigenic protein derived from an influenza virus. In one embodiment, the peptide contained in the second recombinant capsid fusion peptide is derived from a human or avian influenza virus protein selected from the group consisting of M1, M2, hHA, NA, NP, PB1, PB2, PA, and NP2. In one embodiment of the present invention, the peptide contained in the second recombinant capsid fusion peptide is derived from influenza virus proteins HA or NP. In some embodiments the peptide contained in the second recombinant capsid fusion peptide is NP. In other embodiments the peptide contained in the second recombinant capsid fusion peptide is HA. In some embodiments the HA peptide is derived from H1, H2, H3, H4, H5, H6, H7, H8, H9, H10, H11, H12, H13, H14, or H15.

In still another embodiment of the present invention, a composition is provided comprising a virus or virus like particle, wherein the virus or virus like particle comprises a capsid protein derived from a plant virus fused to i) at least one conserved peptide from an influenza virus, and ii) at least one additional isolated influenza viral peptide, wherein the capsid fusion peptides are capable of assembly, in vivo or in vitro, into virus or virus like particles, and iii) an isolated antigenic peptide derived from an influenza virus.

In still another embodiment, the present invention provides a composition comprising a mixture of virus or virus like particles, wherein the mixture comprises i) a first virus or virus like particle containing at least one peptide from an influenza virus, and ii) at least one second virus or virus like particle containing at least one different influenza viral peptide than that contained in the first virus or virus like particle, and iii) an isolated antigenic peptide derived from an influenza virus. In one embodiment, the influenza peptides are fused to a capsid protein derived from a plant virus.

In some aspects of the present invention, the compositions can be utilized in a vaccine strategy to induce an immune response in a human or animal. The compositions can be combined with an adjuvant and administered in an effective amount to a human or animal in order to elicit an immune response. In other embodiments, the compositions are administered without an adjuvant to a human or animal. In some embodiments the composition includes immuno-stimulatory nucleic acid(s), such as CpG sequences. In certain embodiment the immuno-stimulatory nucleic acid(s) can be encapsulated into the virus like particles.

Embodiments of the present invention include wherein the compositions can be administered to a human or animal in a substantially purified form, for example, substantially free of host cell proteins. In other embodiment, the compositions can be administered to a human or animal in a partially purified form, for example, in a form that includes host cell proteins, which can be plant cell proteins.

In another aspect of the present invention, a method of producing a composition for use in an influenza vaccine in a human or animal is provided comprising:
i) providing a first nucleic acid encoding a plant virus capsid protein sequence operably linked to an influenza viral peptide sequence, and expressing the first nucleic acid in a host cell to produce a capsid fusion peptide;
ii) assembling the capsid fusion peptide to form a virus or virus like particle;
iii) providing at least one second nucleic acid encoding at least one antigenic protein or protein fragment derived from an influenza virus strain, and expressing the second nucleic acid in a host cell to produce the antigenic protein or protein fragment;
iv) isolating and purifying the antigenic protein or protein fragment; and
v) combining the virus or virus like particle and the isolated antigenic protein or protein fragment to form a composition capable of administration to a human or animal.

In some embodiments the virus or virus like particle is produced in a plant host, for example, in whole plants or plant cell cultures. In other embodiments, the virus or virus like particle is produced in a Pseudomonas fluorescens host cell. In other embodiments, the capsid fusion peptide is expressed in a host cell such as a plant or Pseudomonas fluorescens cell and the virus or virus like particle is assembled in vitro. In one embodiment, the antigenic protein or protein fragment can be produced in a eukaryotic cell, such as in whole plants or plant cell cultures. In additional embodiments the antigenic protein or protein fragment can be produced in any prokaryotic cell, for example, in E. coli or Pseudomonas fluorescens. In some embodiments the capsid fusion peptide and the antigenic protein or protein fragment are co-expressed in the same eukaryotic cell, and the capsid fusion peptide assembles in vivo to form a virus or virus like particle. In other embodiments the capsid fusion peptide and the antigenic protein or protein fragment are co-expressed in the same prokaryotic cell, such as a Pseudomonas fluorescens cell, and the capsid fusion peptide assembles in vivo to form a virus like particle.

### BRIEF DESCRIPTION OF THE FIGURES

FIG. 1 shows schematic drawing of the influenza vaccine comprising virus or virus like particles displaying influenza virus epitopes and influenza virus protein or protein fragment antigens covalently linked to the VLP. Encapsidation of immuno-stimulatory nucleic acid sequences (CpGs) in the particle is also shown.

FIG. 2 shows schematic drawing of covalent attachment of influenza virus protein or protein fragment antigens to the virus or virus like particle.

FIG. 3 schematic drawing of encapsidation of immuno-stimulatory nucleic acid sequences (CpGs) in the VLP during VLP assembly.

FIG. 4 shows expression of CCMV129 CP fused with M2e-1 influenza virus peptide in Pseudomonas fluorescens as detected by SDS-PAGE stained by Simply blue safe stain (Invitrogen).

FIG. 5 shows expression of CCMV129 CP fused with M2e-2 influenza virus peptide in Pseudomonas fluorescens as detected by SDS-PAGE stained by Simply blue safe stain (Invitrogen).

FIG. 6 shows expression of CCMV129 CP fused with NP55-69 influenza virus peptide in Pseudomonas fluorescens as detected by SDS-PAGE stained by Simply blue safe stain (Invitrogen).

FIG. 7 shows expression of CCMV129 CP fused with NP147-158 influenza virus peptide in Pseudomonas fluorescens as detected by SDS-PAGE stained by Simply blue safe stain (Invitrogen).

FIG. 8 shows expression of CCMV129 CP fused with HA91-108 influenza virus peptide in Pseudomonas fluorescens as detected by SDS-PAGE stained by Simply blue safe stain (Invitrogen).

FIG. 9 shows expression and purification of CCMV129 CP fused with M2e-1 influenza virus peptide in Pseudomonas fluorescens as detected by SDS-PAGE stained by Simply blue safe stain (Invitrogen).

FIG. 10 shows expression of CCMV129 CP fused with M2e-1 influenza virus peptide in Pseudomonas fluorescens as detected by western blotting with anti-CCMV and anti-M2 antibodies 14B. The M2e peptide is recognized by anti-M2 antibodies.

FIG. 11 shows expression of CPMV fused with M2e-1 influenza virus peptide in plants as detected by SDS-PAGE and western blotting with anti-CPMV and anti-M2 antibodies 14B. The M2e peptide is recognized by anti-M2 antibodies.

FIG. 12 shows the sequence of an HA protein from an H5N1 isolate comprising signal peptide, HA1 and HA2, trans-membrane domain, and cytoplasmic tail indicated.

FIG. 13 shows the structure of an H5N1 HA monomer.

FIG. 14 shows schematic drawing of PVX-based viral vectors for expression of influenza proteins or protein fragments in plants.

FIG. 15 shows schematic drawing of plant virus vector-based system for production of influenza virus proteins in plants. The plant virus vector engineered to express influenza virus proteins or protein fragments can be delivered to plants by mechanical inoculation as plasmid DNA, viral RNA, or by Agrobacterium-mediated delivery.

### DETAILED DESCRIPTION

### I. Capsid Fusion Peptides

The present invention utilizes at least one peptide derived from an influenza virus fused to a capsid protein derived from a plant virus forming a recombinant capsid fusion peptide. The recombinant capsid fusion peptide is capable of assembly to form a virus or virus like particle that does not contain host cell plasma membranes.

The recombinant capsid fusion peptide can contain influenza virally derived peptides. In embodiments of the current invention, the recombinant capsid fusion peptide contains a peptide derived from an influenza viral protein. In additional embodiments, the peptide is derived from a conserved peptide, derivate or homologous peptide thereof. The conserved peptide can be derived from an M2, HA, or NP protein. In some embodiments, one, more than one, or combinations of conserved peptides, or derivatives or homologs thereof, derived from M2, HA, or NP can be fused to the capsid protein. A derivative or homolog is generally considered to be an amino acid sequence that is at least about at least 75, 80, 85, 90, 95, 98 or 99% identical with a reference sequence.

a. Human and/or Avian Influenza Derived Peptides

In one embodiment of the present invention, a peptide derived from a human and/or avian influenza virus is genetically fused with a capsid protein derived from a plant virus. Human and avian influenza viral protein sequences are well known in the art. For example, the National Center for Biotechnology Information maintains an Influenza Resource Database containing nucleic acid sequences encoding proteins, and amino acid sequences, from isolated strains of human and avian influenza virus. The database is available at http://www.ncbi.nlm.nih.gov/genomes/FLU/FLU.html.

The peptide selected for insertion into the plant viral capsid protein can be derived from the amino acid sequence of full length influenza virus proteins. In other embodiments, the peptide selected for insertion comprises at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more amino acids in length. The peptide selected can be at least 75, 80, 85, 90, 95, 98 or 99% homologous to an antigenic peptide comprising at least 4, 5, 6, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more amino acids from within the influenza protein from which it is derived.

Preferably, the influenza peptide selected for insertion comprises an epitope capable of eliciting an immune response in a human or animal. Determination of epitopes is well known in the art. For example, a peptide selected for insertion into the plant viral capsid protein can be tested to determine if it is capable of eliciting an immune response by administering the selected peptide to an animal such as a mouse, rabbit, goat, or monkey, and subsequently testing serum from the animal for the presence of antibodies to the peptide. In other embodiments, the influenza derived antigenic peptide can be altered to improve the characteristics of the insert, such as, but not limited to, improved expression in the host, enhanced immunogenicity, and improved covalent binding properties.

b. Influenza M2 Peptide

The influenza M2 protein is a 97 amino acid membrane protein. The protein has 24 amino acids which are exposed extracellularly at the N-terminus, 19 amino acids which span the lipid bilayer, and 54 residues which are located on the cytoplasmic side of the membrane.

In one embodiment, the M2 peptide utilized in the present invention is derived from a 97 amino acid sequence of an influenza virus capable of infecting a human or bird. The derived peptide can comprise the entire 97 amino acid sequence, or be a subset thereof comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 97 amino acids chosen from within the 97 amino acid sequence. The peptide selected can be at least 75, 80, 85, 90, 95, 98 or 99% homologous to the M2 antigenic peptide sequence comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 97 amino acids.

Additional embodiments of the present invention include the M2 peptide utilized in the present invention is derived from the amino acid extra-cellular domain. Embodiments of the present invention include wherein the M2 peptide utilized in the present invention is the 23 amino acid extracellular domain sequence M2e-1 (SEQ ID No: 1, Table 1) derived from the universally conserved M2 sequence. In another embodiment, the M2 peptide utilized in the present invention is comprised of an amino acid subset of the M2e-1 peptide comprising at least 4, 5, 6, 7, 8, 9, 10, 11, 12, 13, 14, 15, 16, 17, 18, 19, 20, 21, 22, or 23 amino acids chosen from within the M2e-1 peptide. The peptide selected can be at least 75, 80, 85, 90, 95, 98 or 99% homologous to the M2e-1 peptide sequence comprising at least 4, 5, 6, 7, 8, 9, 10, 11, 12, 13, 14, 15, 16, 17, 18, 19, 20, 21, 22, or 23 amino acids of SEQ ID No: 1.

In other embodiments, the M2 peptide utilized in the present invention is derived from the 23 amino acid extracellular domain sequence M2e-2 (SEQ ID No: 2, Table 1). In another embodiment, the M2 peptide utilized in the present invention is comprised of an amino acid subset of the M2e-2 peptide comprising at least 4, 5, 6, 7, 8, 9, 10, 11, 12, 13, 14, 15, 16, 17, 18, 19, 20, 21, 22, or 23 amino acids chosen from within the M2e-2 peptide. The peptide selected can be at least 75, 80, 85, 90, 95, 98 or 99% homologous to the M2e-2 peptide sequence comprising at least 4, 5, 6, 7, 8, 9, 10, 11, 12, 13, 14, 15, 16, 17, 18, 19, 20, 21, 22, or 23 amino acids of SEQ ID No: 2.

In another embodiment, the M2 peptide utilized in the present invention is derived from the 22 amino acid extracellular domain sequence M2e-3 (SEQ ID No: 3, Table 1). In another embodiment, the M2 peptide utilized in the present invention is comprised of an amino acid subset of the M2e-3 peptide comprising at least 4, 5, 6, 8, 9, 10, 11, 12, 13, 14, 15, 16, 17, 18, 19, 20, 21, or 22 amino acids chosen from the M2e-3 peptide. The peptide selected can be at least 75, 80, 85, 90, 95, 98 or 99% homologous to the M2e-3 peptide sequence comprising at least 4, 5, 6, 7, 8, 9, 10, 11, 12, 13, 14, 15, 16, 17, 18, 19, 20, 21, or 22 amino acids of SEQ ID No: 3.

In still another embodiment, the M2 peptide utilized in the present invention is derived from the 23 amino acid extracellular domain sequence of influenza strain A/PR/8/34 (H1N1) (SEQ ID No: 4, Table 1). In another embodiment, the M2 peptide utilized in the present invention is comprised of an amino acid subset of the M2 peptide from influenza strain A/PR/8/34 (H1N1) comprising at least 4, 5, 6, 7, 8, 9, 10, 11, 12, 13, 14, 15, 16, 17, 18, 19, 20, 21, 22, or 23 amino acids chosen from the M2 peptide from influenza strain A/PR/8/34 (H1N1). The peptide selected can be at least 75, 80, 85, 90, 95, 98 or 99% homologous to the M2 peptide from influenza strain A/PR/8/34 (H1N1) comprising at least 4, 5, 6, 7, 8, 9, 10, 11, 12, 13, 14, 15, 16, 17, 18, 19, 20, 21, 22, 23 amino acids of SEQ ID No: 4.

In yet another embodiment, the M2 peptide utilized in the present invention is derived from the 23 amino acid extracellular sequence of influenza strain A/Fort Monmouth/1/47 (H1N1) (SEQ ID No: 5, Table 1). In another embodiment, the M2 peptide utilized in the present invention is comprised of an amino acid subset of the M2 peptide from influenza strain A/Fort Monmouth/1/47 (H1N1) comprising at least 4, 5, 6, 7, 8, 9, 10, 11, 12, 13, 14, 15, 16, 17, 18, 19, 20, 21, 22, or 23 amino acids chosen from the M2 peptide from influenza strain A/Fort Monmouth/1/47 (H1N1). The peptide selected can be at least 75, 80, 85, 90, 95, 98 or 99% homologous to the M2 peptide from influenza strain A/Fort Monmouth/1/47 (H1N1) comprising at least 4, 5, 6, 8, 9, 10, 11, 12, 13, 14, 15, 16, 17, 18, 19, 20, 21, 22, or 23 amino acids of SEQ ID No: 5.

In other embodiments, the M2 peptide utilized in the present invention is derived from the 22 amino acid sequence M2e-2(W-) (SEQ ID No: 22, Table 1). In another embodiment, the M2 peptide utilized in the present invention is comprised of an amino acid subset of the M2e-2(W-) peptide comprising at least 4, 5, 6, 7, 8, 9, 10, 11, 12, 13, 14, 15, 16, 17, 18, 19, 20, 21, or 22 amino acids chosen from within the M2e-2(W-) peptide. The peptide selected can be at least 75, 80, 85, 90, 95, 98 or 99% homologous to the M2e-2(W-) peptide sequence comprising at least 4, 5, 6, 7, 8, 9, 10, 11, 12, 13, 14, 15, 16, 17, 18, 19, 20, 21, or 22 amino acids of SEQ ID No: 22.

In still another embodiment, the M2 peptide utilized in the present invention is derived from the 22 amino acid sequence of A/PR/8/34 (H1N1)(W-) (SEQ ID No: 23, Table 1). In another embodiment, the M2 peptide utilized in the present invention is comprised of an amino acid subset of M2-A/PR/8/34 (H1N1)(W-) comprising at least 4, 5, 6, 7, 8, 9, 10, 11, 12, 13, 14, 15, 16, 17, 18, 19, 20, 21, or 22 amino acids chosen from A/PR/8/34 (H1N1)(W-). The peptide selected can be at least 75, 80, 85, 90, 95, 98 or 99% homologous to the A/PR/8/34 (H1N1)(W-) peptide comprising at least 4, 5, 6, 7, 8, 9, 10, 11, 12, 13, 14, 15, 16, 17, 18, 19, 20, 21, 22, 23 amino acids of SEQ ID No: 23.

In yet another embodiment, the M2 peptide utilized in the present invention is derived from the 22 amino acid sequence of A/Fort Monmouth/1/47 (H1N1)(W-) (SEQ ID No: 24, Table 1). In another embodiment, the M2 peptide utilized in the present invention is comprised of an amino acid subset M2-A/Fort Monmouth/1/47 (H1N1)(W-) comprising at least 4, 5, 6, 7, 8, 9, 10, 11, 12, 13, 14, 15, 16, 17, 18, 19, 20, 21, or 22 amino acids chosen from the M2-A/Fort Monmouth/1/47 (H1N1)(W-). The peptide selected can be at least 75, 80, 85, 90, 95, 98 or 99% homologous to the peptide M2-A/Fort Monmouth/1/47 (H1N1)(W-) comprising at least 4, 5, 6, 8, 9, 10, 11, 12, 13, 14, 15, 16, 17, 18, 19, 20, 21, or 22 amino acids of SEQ ID No: 24.

In additional embodiments, the M2 peptide inserted into the plant virus capsid protein can be the entire amino acid sequence selected from the group consisting of SEQ ID Nos: 1-5 and 22-24, or a subset thereof having at least 4, 5, 6, 7, 8, 9, 10, 11, 12, 13, 14, 15, 16, 17, 18, 19, 20, 21, 22, 23, 24 or more amino acids in length. The peptide selected for insertion can be at least 75, 80, 85, 90, 95, 98 or 99% homologous to a peptide at least 4, 5, 6, 7, 8, 9, 10, 11, 12, 13, 14, 15, 16, 17, 18, 19, 20, 21, 22, 23, 24, or more amino acids in length from within the peptide sequences selected from the group consisting of SEQ ID Nos: 1-5 and 22-24.

In other embodiments, any combination of M2 peptides selected from the group consisting of SEQ ID No: 1-5 and 22-24, or a subset thereof having at least 4, 5, 6, 7, 8, 9, 10, 11, 12, 13, 14, 15, 16, 17, 18, 19, 20, 21, 22, 23, 24 or more amino acids in length can be inserted into the plant virus capsid protein. The peptide combinations selected can be at least 75, 80, 85, 90, 95, 98 or 99% homologous to at least 4, 5, 6, 7, 8, 9, 10, 11, 12, 13, 14, 15, 16, 17, 18, 19, 20, 21, 22, 23, 24, or more amino acids selected from the group consisting of SEQ ID No: 1-5 and 22-24.

In additional embodiments, the M2 influenza derived antigenic peptide can be altered to improve the characteristics of the insert, such as, but not limited to, improved expression in the host, enhanced immunogenicity, and improved covalent binding properties. Embodiments of the present invention include wherein the amino acid tryptophan in SEQ ID No: 1, 2, 4, or 5 is removed or replaced with any amino acid that is not tryptophan.

TABLE 1
M2 peptide sequences

| | | |
|---|---|---|
| Sequence | Name | Seq. ID. No. |

| | | |
|---|---|---|
| SLLTEVETPIRNEWGCRCNDSSD | M2e-1 | SEQ ID No: 1 |
| SLLTEVETPIRNEWECRCNGSSD | M2e-2 | SEQ ID No: 2 |
| SLLTEVETPIRNEGCRCNDSSD | M2e-3 | SEQ ID No: 3 |
| SLLTEVETPIRNEWGCRCNGSSD | M2e-A/PR/8/34 (H1N1) | SEQ ID No: 4 |
| SLLTEVETPTKNEWECRCNDSSD | M2e-A/Fort Monmouth/1/47 | SEQ ID No: 5 |
| | (H1N1) | |
| SLLTEVETPIRNEECRCNGSSD | M2e-2(W-) | SEQ ID No: 22 |
| SLLTEVETPIRNEGCRCNGSSD | M2e-A/PR/8/34 (H1N1)(W-) | SEQ ID No: 23 |
| SLLTEVETPTKNEECRCNDSSD | M2e-A/Fort Monmouth/1/47 | SEQ ID No: 24 |
| | (H1N1)(W-) | |

The present invention also provides novel M2 derived peptides. In one embodiment, the novel M2 peptide M2e-3 comprising SEQ ID No: 3 is provided. In one embodiment, amino acid sequences at least 70, 75, 80, 90, 95, 98 or 99% homologous to SEQ ID No: 3 are provided. In another embodiment, a peptide comprising at least, 4, 5, 6, 7, 8, 9, 10, 11, 12, 13, 14, 15, 16, 17, 18, 19, 20, 21, or 22 amino acids derived from SEQ ID No: 3 is provided. The M2e-3 peptide is derived from the M2e-1 peptide, wherein the amino acid tryptophan has been removed. The removal of the tryptophan provides for increased assembly of certain capsid fusion peptides, while not adversely affecting the immunogenicity of the peptide. In one embodiment, the M2e-3 peptide is inserted into a plant viral capsid protein. Embodiments of the present invention include wherein the M2e-3 peptide is inserted into a capsid protein derived from CCMV or CPMV.

In one embodiment, the novel M2 peptide M2e-2(W-) comprising SEQ ID No: 22 is provided. In one embodiment, amino acid sequences at least 70, 75, 80, 90, 95, 98 or 99% homologous to SEQ ID No: 22 are provided. In another embodiment, a peptide comprising at least, 4, 5, 6, 7, 8, 9, 10, 11, 12, 13, 14, 15, 16, 17, 18, 19, 20, 21, or 22 amino acids derived from SEQ ID No: 22 is provided. The M2e-2(W-) peptide is derived from the M2e-2 peptide, wherein the amino acid tryptophan has been removed. In one embodiment, the M2e-2(W-) peptide is inserted into a capsid protein derived from a plant virus. Embodiments of the present invention include wherein the M2e-2(W-) peptide is inserted into a capsid protein derived from CCMV or CPMV.

In one embodiment, the novel M2 peptide M2e-A/PR/8/34 (H1N1)(W-) comprising SEQ ID No: 23 is provided. In one embodiment, amino acid sequences at least 70, 75, 80, 90, 95, 98 or 99% homologous to SEQ ID Nos: 23 are provided. In another embodiment, a peptide comprising at least, 4, 5, 6, 7, 8, 9, 10, 11, 12, 13, 14, 15, 16, 17, 18, 19, 20, 21, or 22 amino acids derived from SEQ ID No: 23 is provided. The M2e-A/PR/8/34 (H1N1)(W-) peptide is derived from the M2e-A/PR/8/34 (H1N1) peptide, wherein the amino acid tryptophan has been removed. In one embodiment, the M2e-A/PR/8/34 (H1N1)(W-) peptide is inserted into a capsid protein derived from a plant virus. Embodiments of the present invention include wherein the M2e-A/PR/8/34 (H1N1)(W-) peptide is inserted into a capsid protein derived from CCMV or CPMV.

In one embodiment, the novel M2 peptide M2e-A/Fort Monmouth/1/47 (H1N1)(W-) comprising SEQ ID No: 24 is provided. In one embodiment, amino acid sequences at least 70, 75, 80, 90, 95, 98 or 99% homologous to SEQ ID No: 24 are provided. In another embodiment, a peptide comprising at least, 4, 5, 6, 7, 8, 9, 10, 11, 12, 13, 14, 15, 16, 17, 18, 19, 20, 21, or 22 amino acids derived from SEQ ID No: 24 is provided. The peptide is derived from the M2e-A/Fort Monmouth/1/47 (H1N1) peptide, wherein the amino acid tryptophan has been removed. In one embodiment, the M2e-A/Fort Monmouth/1/47 (H1N1)(W-) peptide is inserted into a capsid protein derived from a plant virus. Embodiments of the present invention include wherein the M2e-A/Fort Monmouth/1/47 (H1N1)(W-) peptide is inserted into a capsid protein derived from CCMV or CPMV.

Novel compositions comprising a capsid fusion peptide comprising a capsid protein derived from a virus, including a plant virus, fused to a peptide selected from the group consisting of SEQ ID Nos: 3, 22, 23, and 24 are also provided.

b. HA Protein

Influenza virus hemagglutinin (HA) is a type I transmembrane glycoprotein that appears on influenza virus particles as homotrimers with multiple folding domains. The monomer has six intrachain disulfide bonds and seven N-linked glycans in the N-terminal ectodomain, a transmembrane domain and a cytosolic tail. Wilson et al. (1981) “Structure of the haemagglutinin membrane glycoprotein of influenza virus at 3 A° resolution,” Nature 289:366-373; Wiley D. C. and J J. Skehel (1987) “The structure and function of hemagglutinin membrane glycoprotein of influenza virus,” Annu. Rev. Biochem. 56:365-394. The crystal structure of the ectodomain of the proteolytically activated trimers reveals a 135 A° long trimeric spike protein in which each subunit has two major domains: a globular NH₂-terminal top domain and a COOH-terminal domain which forms the stem of the spike protein. The stem region contains the fusion peptides known to be involved in the membrane fusion activity of the protein. Wilson et al. (1981) “Structure of the haemagglutinin membrane glycoprotein of influenza virus at 3 A° resolution,” Nature 289:366-373; Wiley D. C. and J J. Skehel (1987) “The structure and function of hemagglutinin membrane glycoprotein of influenza virus,” Annu. Rev. Biochem. 56:365-394.

In one embodiment, the HA peptide utilized in the present invention is derived from a HA protein contained in an influenza virus selected from the group of fifteen classes of hemagglutinin antigens H1-H15. The derived peptide can comprise the entire HA amino acid sequence, or be a subset thereof comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 331, 332, or 333 or more amino acids chosen from within the HA amino acid sequence. The peptide selected can be at least 75, 80, 85, 90, 95, 98 or 99% homologous to the HA antigenic peptide sequence of the influenza protein comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 331, 332, 333 or more amino acids chosen from within the HA amino acid sequence from which it is derived.

Additional embodiments of the present invention include the HA peptide utilized in the present invention is derived from an influenza virus capable of infecting a human or bird. Embodiments of the present invention include wherein the HA peptide inserted into the plant virus capsid protein utilized in the present invention is derived from an H3 subtype. In additional embodiments the HA peptide can be derived from the 333 amino acid HA protein of influenza strain A/Texas/1/77 (H3N2) (SEQ ID No: 6, Table 2). CB Smith et al. (2002) “Molecular epidemiology of influenza A(H3N2) virus re-infections,” J. Infect. Dis. 185 (7):980-985. In another embodiment, the HA peptide utilized for insert in the plant capsid protein can comprise the entire HA amino acid sequence of SEQ ID No: 6, or be a subset thereof comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 331, 332, 333 or more amino acids chosen from within the HA amino acid sequence of SEQ ID No: 6. The peptide selected can be at least 75, 80, 85, 90, 95, 98 or 99% homologous to the HA antigenic peptide sequence comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 331, 332, 333 or more amino acids of SEQ ID No: 6.

Additional embodiments of the present invention include the HA peptide utilized in the present invention is derived from the 18 amino acid sequence HA91-108-A/Texas/1/77 (H3N2) (SEQ ID No: 7, Table 2) or a subset thereof having at least 4, 5, 6, 7, 8, 9, 10, 11, 12, 13, 14, 15, 16, 17 or 18 amino acids in length derived from HA amino acids 91-108 of the influenza A/Texas/1/77 (H3N2) strain. The peptide selected can be at least 75, 80, 85, 90, 95, 98 or 99% homologous to the HA antigenic peptide sequence comprising the 18 amino acid sequence HA91-108-A/Texas/1/77 (H3N2) (SEQ ID No: 7, Table 2) or a subset thereof having at least 4, 5, 6, 7, 8, 9, 10, 11, 12, 13, 14, 15, 16, 17, or 18 amino acids in length derived from HA amino acids 91-108 of the influenza A/Texas/1/77 (H3N2) strain.

In additional embodiments, the HA peptide inserted into the plant virus capsid protein can be the entire amino acid sequence selected from the group consisting of SEQ ID Nos: 6 and 7, or a subset thereof having at least at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 331, 332, 333 or more amino acids in length. In other embodiments, any combination of HA peptides selected from the group consisting of SEQ ID Nos: 6 and 7, or a subset thereof having at least 4, 5, 6, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 331, 332, 333 or more amino acids in length can be inserted into the plant virus capsid protein. The peptide selected can be at least 75, 80, 85, 90, 95, 98 or 99% homologous to the HA antigenic peptide sequence from the selected peptide derived from the group consisting of SEQ ID Nos: 6-7.

In additional embodiments, the influenza derived HA antigenic peptide can be altered to improve the characteristics of the insert, such as, but not limited to, improved expression in the host, enhanced immunogenicity, and improved covalent binding properties.

TABLE 2
HA peptide sequences

| | | |
|---|---|---|
| Sequence | Name | Seq. ID. No. |

| | | |
|---|---|---|
| QNLPGNDNSTATLCLGH | HA-A/Texas/1/77 | SEQ ID No: 6 |
| HAVPNGTLVKTITNDQI | (H3N2) | |
| EVTNATELVQSSSTGRI | | |
| CDSPHRILDGKNCTLID | | |
| ALLGDPHCDGFQNEKWD | | |
| LFVERSKAFSNCYPYDV | | |
| PDYASLRSLVASSGTLE | | |
| FINEGFNWTGVTQNGGS | | |
| YACKRGPDNGFFSRLNW | | |
| LYKSESTYPVLNVTMPN | | |
| NGNFDKLYIWGVHHPST | | |
| DKEQTNLYVQASGRVTV | | |
| STKRSQQTIIPNVGSRP | | |
| WVRGLSSRISIYWTIVK | | |
| PGDILLINSNGNLIAPR | | |
| GYFKIRTGKSSIMRSDA | | |
| PIGTCSSECITPNGSIP | | |
| NDKPFQNVNKITYGACP | | |
| KYVKQNTLKLATGMRNV | | |
| PEKQTRGLFG | | |
| SKAFSNCYPYDVPDYASL | HA91-108- | SEQ ID No: 7 |
| | A/Texas/1/77 (H3N2) | |

c. NP Protein

Influenza virus nucleoprotein (NP) is a helical nucleoprotein closely associated with the viral single stranded RNA genome. The influenza NP protein is rich in arginine, glycine and serine residues and has a net positive charge at neutral pH. The influenza type A NP protein is generally composed of a polypeptide of 498 amino acids in length, while the influenza B and C viruses, the length of the homologous NP polypeptide is generally 560 and 565 residues, respectively. See Londo et al. (1983) “Complete nucleotide sequence of the nucleoprotein gene of influenza B virus,” Journal of Virology 47:642-648; S. Nakada et al. (1984) “Complete nucleotide sequence of the influenza C/California/78 virus nucleoprotein gene,” Virus Research 1: 433-441. Alignment of the predicted amino acid sequences of the NP genes of the three influenza virus types reveals significant similarity among the three proteins, with the type A and B NPs showing the highest degree of conservation. See Portela and Digard (2002) “The influenza virus nucleoprotein: a multifunctional RNA-binding protein pivotal to virus replication 2002,” JGV 83:723-734. Phylogenetic analysis of virus strains isolated from different hosts reveals that the NP gene is relatively well conserved, with a maximum amino acid difference of less than 11%. See Shu et al. (1993) “Analysis of the evolution and variation of the human influenza A virus nucleoprotein gene from 1933 to 1990,” Journal of Virology 67:2723-2729.

In one embodiment, the NP peptide utilized in the present invention is derived from an NP protein contained in an influenza type A, B, or C virus. The derived peptide can comprise the entire NP amino acid sequence, or be a subset thereof comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 350, 360, 375, 380, 390, 400, 410, 425, 435, 445, 450, 460, 470, 480, 490, 495, 498 or more amino acids chosen from within the NP amino acid sequence. The peptide selected can be at least 70, 75, 80, 85, 90, 95, 98 or 99% homologous to the NP antigenic peptide sequence of the influenza protein comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 320, 325, 330, 350, 360, 375, 380, 390, 400, 410, 425, 435, 445, 450, 460, 470, 480, 490, 495, 498 or more amino acids chosen from within the NP amino acid sequence.

Additional embodiments of the present invention include the NP peptide utilized in the present invention is derived from an influenza virus capable of infecting a human or bird. Embodiments of the present invention include wherein the NP peptide inserted into the plant virus capsid protein utilized in the present invention is derived from the NP protein derived from an influenza Type A virus. The NP protein is derived from the 498 amino acid NP protein of influenza strain A/Texas/1/77 (H3N2) (SEQ ID No: 8, Table 3) or be a subset thereof comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 350, 360, 375, 380, 390, 400, 410, 425, 435, 445, 450, 460, 470, 480, 490, 495, 498 or more amino acids chosen from within the NP amino acid sequence of SEQ ID No: 8. The peptide selected can be at least 70, 75, 80, 85, 90, 95, 98 or 99% homologous to the NP antigenic peptide sequence of the influenza protein comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 320, 325, 330, 350, 360, 375, 380, 390, 400, 410, 425, 435, 445, 446, or more amino acids chosen from within the NP amino acid sequence of SEQ ID No: 8.

Additional embodiments of the present invention include the NP peptide utilized in the present invention is derived from the 15 amino acid sequence NP55-69-A/Texas/1/77 (H3N2) (SEQ ID No: 9, Table 3) or a subset thereof having at least 4, 5, 6, 7, 8, 9, 10, 11, 12, 13, 14, or 15 amino acids in length derived from NP amino acids 55-69 of the influenza A/Texas/1/77 (H3N2) strain. The peptide selected can be at least 70, 75, 80, 85, 90, 95, 98 or 99% homologous to the NP antigenic peptide sequence or a subset thereof having at least 4, 5, 6, 7, 8, 9, 10, 11, 12, 13, 14, 15 amino acids in length derived from NP amino acids 55-69 of the influenza A/Texas/1/77 (H3N2) strain.

In other embodiments, the NP peptide utilized in the present invention is derived from the 12 amino acid sequence NP147-158-A/Texas/1/77 (H3N2) (SEQ ID No: 10, Table 3) or a subset thereof having at least 4, 5, 6, 7, 8, 9, 10, 11, or 12 amino acids in length derived from NP amino acids 147-158 of the influenza A/Texas/1/77 (H3N2) strain. The peptide selected can be at least 70, 75, 80, 85, 90, 95, 98 or 99% homologous to the NP antigenic peptide sequence or a subset thereof having at least 4, 5, 6, 7, 8, 9, 10, 11, or 12 amino acids in length derived from NP amino acids 147-158 of the influenza A/Texas/1/77 (H3N2) strain.

In additional embodiments, the NP peptide inserted into the plant virus capsid protein can be the entire amino acid sequence selected from the group consisting of SEQ ID Nos: 8-10, or a subset thereof having at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 320, 325, 330, 350, 360, 375, 380, 390, 400, 410, 425, 435, 445, 450, 460, 470, 480, 490, 495, 498, or more amino acids in length. The peptide selected can be at least 70, 75, 80, 85, 90, 95, 98 or 99% homologous to the NP antigenic peptide sequence of the influenza protein comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 320, 325, 330, 350, 360, 375, 380, 390, 400, 410, 425, 435, 445, 450, 460, 470, 480, 490, 495, 498, or more amino acids chosen from within the NP amino acid sequence selected from the group consisting of SEQ ID Nos: 8-10. In other embodiments, any combination of NP peptides selected from the group consisting of SEQ ID Nos: 8-10, or a subset thereof having 70, 75, 80, 85, 90, 95, 98 or 99% homologous to the NP antigenic peptide sequence of the influenza protein comprising at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 125, 135, 140, 150, 160, 170, 180, 190, 200, 210, 225, 235, 250, 260, 275, 280, 290, 300, 310, 320, 325, 330, 320, 325, 330, 350, 360, 375, 380, 390, 400, 410, 425, 435, 445, 450, 460, 470, 480, 490, 495, 498 or more amino acids in length derived from the group consisting of SEQ ID Nos: 8-10 can be inserted into the plant virus capsid protein.

In additional embodiments, the influenza NP derived antigenic peptide can be altered to improve the characteristics of the insert, such as, but not limited to, improved expression in the host, enhanced immunogenicity, and improved covalent binding properties.

TABLE 3
NP amino acid sequences

| | | SEQ ID |
|---|---|---|
| Sequence | Name | NO: |
| MASQGTKRSYEQMETDG | NP-A/Texas/1/77 | SEQ ID No: 8 |
| ERQNATEIRASVGKMID | (H3N2) | |
| GIGRFYIQMCTELKLSD | | |
| YEGRLIQNSLTIERMVL | | |
| SAFDERRNKYLEEHPSA | | |
| GKDPKKTGGPIYKRVDG | | |
| KWMRELVLYDKEEIRRI | | |
| WRQANNGDDATRGLTHM | | |
| MIWHSNLNDTTYQRTRA | | |
| LVRTGMDPRMCSLMQGS | | |
| TLPRRSGAAGAAVKGIG | | |
| TMVMELIRMIKRGINDR | | |
| NFWRGENGRKTRSAYER | | |
| MCNILKGKFQTAAQRAM | | |
| MDQVRESRNPGNAEIED | | |
| LIFSARSALILRGSVAH | | |
| KSCLPACVYGPAVASGY | | |
| DFEKEGYSLVGIDPFKL | | |
| LQNSQVYSLIRPNENPA | | |
| HKSQLVWMACHSAAFED | | |
| LRLLSFIRGTKVSPRGK | | |
| LSTRGVQIASNENMDTM | | |
| ESSTLELRSRYWAIRTR | | |
| SGGNTNQQRASAGQISV | | |
| QPTFSVQRNLPFDKSTI | | |
| MAAFTGNTEGRTSDMRA | | |
| EIIRMMEGAKPEEVSFR | | |
| GRGVFELSDEKATNPIV | | |
| PSFDMSNEGSYFFGDNA | | |
| EEYDN | | |
| RLIQNSLTIERMVLS | NP55-69- | SEQ ID No: 9 |
| | A/Texas/1/77 (H3N2) | |
| TYQRTRALVRTG | NP147-158- | SEQ ID No: |
| | A/Texas/1/77 (H3N2) | 10 |

d. Capsid Protein

The present invention utilizes capsid proteins derived from plant viruses to construct capsid fusion peptides. One potential advantage to the use of capsid proteins from a plant virus is the reduced potential for adverse reactions when administered to a human or animal, while maintaining the advantageous form of a viral particle to present the influenza epitope.

In additional embodiments, the capsid protein will be derived from plant viruses selected from members of any one of the taxa that are specific for at least one plant host.

Viral taxonomies recognize the following taxa of encapsidated-particle entities: Group I Viruses, i.e. the dsDNA viruses; Group II Viruses, i.e. the ssDNA viruses; Group III Viruses, i.e. the dsRNA viruses; Group IV Viruses, i.e. the ssRNA (+)-stranded viruses with no DNA stage; Group V Viruses, i.e. the ssRNA (−)-stranded viruses; Group VI Viruses, i.e. the RNA retroid viruses, which are ssRNA reverse transcribing viruses; Group VII Viruses, i.e. the DNA retroid viruses, which are dsDNA reverse transcribing viruses; Deltaviruses; Viroids; and Satellite phages and Satellite viruses, excluding Satellite nucleic acids and Prions.

Members of these taxa are well known to one of ordinary skill in the art and are reviewed in: H. V. Van Regenmortel et al. (eds.), Virus Taxonomy: Seventh Report of the International Committee on Taxonomy of Viruses (2000) (Academic Press/Elsevier, Burlington Mass., USA); the Virus Taxonomy web-page of the University of Leicester (UK) Microbiology & Immunology Department at http://wwwmicro.msb.le.ac.uk/3035/Virusgroups.html; and the on-line “Virus” and “Viroid” sections of the Taxonomy Browser of the National Center for Biotechnology Information (NCBI) of the National Library of Medicine of the National Institutes of Health of the US Department of Health & Human Services (Washington, D.C., USA) at http://www.ncbi.nlm.nih.gov/Taxonomy/tax.html.

The amino acid sequence of the capsid may be selected from the capsids of any members of any of these taxa that are infectious to plants. Amino acid sequences for capsids of the members of these taxa may be obtained from sources, including, but not limited to, e.g.: the on-line “Nucleotide” (Genbank), “Protein,” and “Structure” sections of the PubMed search facility offered by the NCBI at http://www.ncbi.nlm.nih.gov/entrez/query.fcgi.

Viruses can be classified into those with helical symmetry or icosahedral symmetry. Generally recognized capsid morphologies include: icosahedral (including icosahedral proper, isometric, quasi-isometric, and geminate or “twinned”), polyhedral (including spherical, ovoid, and lemon-shaped), bacilliform (including rhabdo- or bullet-shaped, and fusiform or cigar-shaped), and helical (including rod, cylindrical, and filamentous); any of which may be tailed and/or may contain surface projections, such as spikes or knobs. In one embodiment of the invention, the amino acid sequence of the capsid is selected from the capsids of viruses classified as having any morphology.

In one embodiment, the capsid is derived from a rod shaped plant virus. Additional embodiments of the present invention include the capsid is a rod shaped viral capsid derived from the group selected from Tobacco Mosaic Virus (TMV) and Potato Virus X (PVX). TMV consists of a single plus-sense genomic RNA (6.5 kb) encapsidated with a unique coat protein (17.5 kDa) which results in rod-shaped particles (300 nm). A wide host range of tobacco mosaic virus allows one to use a variety of plant species as production and delivery systems. It has previously been shown that foreign genes inserted into this vector can produce high levels of protein. Yusibov et al. (1995) “High-affinity RNA-binding domains of alfalfa mosaic virus coat protein are not required for coat protein-mediated resistance,” Proc. Natl. Acad. Sci. U.S. 92:8980-8984. Potato Virus X are filamentous, non enveloped; usually flexuous viruses with a clear modal length of 515 nm and 13 nm wide. The capsid structure forms a basic helix with a pitch of 3.4 nm. Varma A, Gibbs A J, Woods R D, Finch J T (1968) “Some observations on the structure of the filamentous particles of several plant viruses,” J Gen Virol. 2(1):107-14. In other embodiments, the capsid protein is derived from a plant virus that is not TMV.

In one embodiment, the capsid has an icosahedral morphology. Generally, viral capsids of icosahedral viruses are composed of numerous protein sub-units arranged in icosahedral (cubic) symmetry. Native icosahedral capsids can be built up, for example, with 3 subunits forming each triangular face of a capsid, resulting in 60 subunits forming a complete capsid. Representative of this small viral structure is e.g. bacteriophage ØX174. Many icosahedral virus capsids contain more than 60 subunits. Many capsids of icosahedral viruses contain an antiparallel, eight-stranded beta-barrel folding motif. The motif has a wedge-shaped block with four beta strands (designated BIDG) on one side and four (designated CHEF) on the other. There are also two conserved alpha-helices (designated A and B), one is between betaC and betaD, the other between betaE and betaF.

In one embodiment the icosahedral plant virus species will be a plant-infectious virus species that is or is a member of any of the Bunyaviridae, Reoviridae, Rhabdoviridae, Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In one embodiment, the icosahedral plant virus species is a plant-infectious virus species that is or is a member of any of the Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In specific embodiments, the icosahedral plant virus species is a plant infectious virus species that is or is a member of any of the Caulimoviridae, Geminiviridae, Comoviridae, Sobemovirus, Tombusviridae, or Bromoviridae. In other embodiments the icosahedral plant virus species will be a plant-infectious virus species that is or is a member of any of the Comoviridae, Sobemovirus, Tombusviridae, or Bromoviridae. In additional embodiments the capsid is derived from an Ilarvirus or an Alfamovirus. In additional embodiments the capsid is derived from a Tobacco streak virus, Alfalfa mosaic virus (AMV), or Brome Mosaic Virus (BMV). In other embodiments the icosahedral plant virus species can be a plant-infectious virus species that is a member of the Comoviridae or Bromoviridae family. Embodiments of the present invention include wherein the viral capsid is derived from a Cowpea Mosaic Virus (CPMV) or a Cowpea Chlorotic Mottle Virus (CCMV).

Embodiments of the present invention include wherein the capsid protein utilized in the present invention is derived from a CCMV capsid protein. More specifically, the capsid protein is derived from the CCMV capsid amino acid sequence represented by SEQ ID No: 11 (Table 4). In other embodiments the capsid protein utilized in the present invention can be the entire amino acid sequence of the CCMV large capsid protein, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, or more amino acids selected from SEQ ID No: 11. The capsid protein selected can be at least 75, 80, 85, 90, 95, 98, or 99% homologous to the amino acid sequence of the CCMV large capsid protein, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, or more amino acids selected from SEQ ID No: 11. In other embodiments, the capsid protein can be altered to improve the characteristics of the capsid fusion peptide, such as, but not limited to, improved expression in the host, enhanced immunogenicity, improved covalent binding properties, or improved folding or reassembly.

In other embodiments, the capsid protein utilized in the present invention is derived from the CPMV small capsid protein (S CPMV Capsid). More specifically, the capsid protein is derived from the S CPMV capsid amino acid sequence represented by SEQ ID No: 12 (Table 4). In other embodiments, the capsid protein utilized in the present invention can be the entire amino acid sequence of the CPMV small capsid protein, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 210, 213 or more amino acids selected from SEQ ID No: 12. The capsid protein selected can be at least 75, 80, 85, 90, 95, 98, or 99% homologous to the amino acid sequence of the CPMV small capsid protein, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 210, 213 or more amino acids selected from SEQ ID No: 12. In other embodiments, the capsid protein can be altered to improve the characteristics of the capsid fusion peptide, such as, but not limited to, improved expression in the host, enhanced immunogenicity, improved covalent binding properties, or improved folding or reassembly.

In another embodiment, the capsid protein utilized in the present invention is derived from the CPMV large capsid protein (L CPMV Capsid). More specifically, the capsid protein is derived from the L CPMV capsid amino acid sequence represented by SEQ ID No: 13 (Table 4). In other embodiments, the capsid protein utilized in the present invention can be the entire amino acid sequence of the CPMV large capsid protein, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 210, 225, 240, 250, 265, 275, 285, 290, 300, 310, 320, 330, 340, 350, 360, 370, 374 or more amino acids selected from SEQ ID No: 12. The capsid protein selected can be at least 75, 80, 85, 90, 95, 98, or 99% homologous to the amino acid sequence of the CPMV large capsid protein, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 210, 225, 240, 250, 265, 275, 285, 290, 300, 310, 320, 330, 340, 350, 360, 370, 374 or more amino acids selected from SEQ ID No: 13. In other embodiments, the capsid protein can be altered to improve the characteristics of the capsid fusion peptide, such as, but not limited to, improved expression in the host, enhanced immunogenicity, improved covalent binding properties, or improved folding or reassembly.

TABLE 4

Plant Viral Capsid Amino
Acid and Nucleotide Sequences.

| | | |
|---|---|---|
| Sequence | Name | SEQ ID No: |

| | | |
|---|---|---|
| MSTVGTGKLTRAQRRAAARKNKRNTRVVQPV | CCMV | SEQ ID No: 11 |
| IVEPIASGQGKAIKAWTGYSVSKWTASCAAA | Capsid | |
| EAKVTSAITISLPNELSSERNKQLKVGRVLL | | |
| WLGLLPSVSGTVKSCVTETQTTAAASFQVAL | | |
| AVADNSKDVVAAMYPEAFKGITLEQLTADLT | | |
| IYLYSSAALTEGDVIVHLEVEHVRPTFDDSF | | |
| TPVY | | |
| GPVCAEASDVYSPCMIASTPPAPFSDVTAVT | S CPMV | SEQ ID No: 12 |
| FDLINGKLITPVGDDNWNTHIYNPPIMNVLR | Capsid | |
| TAAWKSGTIHVQLNVRGAGVKRADWDGQVFV | | |
| YLRQSMNPESYDARTFVISQPGSAMLNESFD | | |
| IIGPNSGFEFAESPWANQTTWYLECVATNPR | | |
| QIQQFEVNMRFDPNFRVAGNILMPPF PLST | | |
| ETPPLLKFRFRDIERSKRSVMVGHTATAA | | |
| MEQNLFALSLDDTSSVRGSLLDTKFAQTRVL | L CPMV | SEQ ID No: 13 |
| LSKAMAGGDVLLDEYLYDVVNGQDFRATVAF | Capsid | |
| LRTHVITGKIKVTATTNISDNSGCCLMLAIN | | |
| SGVRGKYSTDVYTIGSQDSMTWNPGCKKNFS | | |
| FTFNPNPCGDSWSAEMISRSRVRMTVICVSG | | |
| WTLSPTTDVIAKLDWSIVNEKCEPTIYHLAD | | |
| CQNWLPLNRWMGKLTFPQGVTSEVRRMPLSI | | |
| GGGAGATQAFLANMPNSWISMWRYFRGELHF | | |
| EVTKMSSPYIKATVTFLIAFGNLSDAFGFYE | | |
| SFPHRIVQFAEVEEKCTLVFSQQEFVTAWST | | |
| QVNPRTTLEADGCPYLYAIIHDSTTGTISGD | | |
| FNLGVKLVGIKDFCGIGSNPGIDGSRLLGAI | | |
| AQ | | |

e. Capsid Fusion Peptide Generation

A nucleic acid encoding a peptide derived from an influenza virus is genetically fused to a nucleic acid encoding a plant viral capsid protein to produce a construct capable of being expressed as a recombinant fusion peptide. The recombinant capsid peptides for use in the present invention can be produced in biological expression systems utilizing well-known techniques in the art. For example, nucleic acid constructs encoding a fusion peptide of a plant viral capsid protein operably linked to at least one antigenic influenza peptide can be introduced into a host cell and expressed. Transcriptional and translational regulatory elements, such as transcriptional enhancer sequences, translational enhancer sequences, promoters, ribosomal entry sites, including internal ribosomal entry sites, activators, translational start and stop signals, transcription terminators, cistronic regulators, polycistronic regulators, tag sequences, such as nucleotide sequence “tags” and “tag” peptide coding sequences, which facilitates identification, separation, purification, or isolation of the expressed recombinant capsid protein fusion peptide, including His-tag, Flag-tag, T7-tag, S-tag, HSV-tag, B-tag, Strep-tag, polyarginine, polycysteine, polyphenylalanine, polyaspartic acid, (Ala-Trp-Trp-Pro)n, thioredoxin, beta-galactosidase, chloramphenicol acetyltransferase, cyclomaltodextrin gluconotransferase, CTP:CMP-3-deoxy-D-manno-octulosonate cytidyltransferase, trpE or trpLE, avidin, streptavidin, T7 gene 10, T4 gp55, Staphylococcal protein A, streptococcal protein G, GST, DHFR, CBP, MBP, galactose binding domain, Calmodulin binding domain, KSI, c-myc, ompT, ompA, pelB, NusA, ubiquitin, hex-histidine, glutathione-S-transferase, GFP, YFP, or analogs of such fluorescent proteins, antibody molecules, hemosylin A, or a known antigen or ligand for a known binding partner useful for purification can be included in the nucleic acid sequence for expression in the host cell.

The nucleic acid coding sequence for the influenza peptide or peptides can be inserted into the nucleic acid coding sequence for the viral capsid protein in a predetermined site. In one embodiment, the influenza peptide is inserted into the capsid coding sequence so as to be expressed as a loop during formation of a virus or virus like particle.

Influenza peptides may be inserted at more than one insertion site in the plant capsid. Thus, influenza peptides may be inserted in more than one surface loop motif of a capsid when the capsid fusion peptides reassemble to form a virus or virus like particle. Alternatively, influenza peptides may also be inserted at multiple sites within a given loop motif when the capsid fusion peptides assemble to form a virus or virus like particle.

In addition, influenza peptides may be inserted within external-facing loop(s) and/or within internal-facing loop(s), i.e. within loops of the capsid that face respectively away from or toward the center of the capsid. Any amino acid or peptide bond in a surface loop of a capsid can serve as an insertion site for the influenza peptide. Typically, the insertion site can be selected at about the center of the loop, i.e. at about the position located most distal from the center of the tertiary structure of the folded capsid peptide. The influenza peptide coding sequence may be operably inserted within the position of the capsid coding sequence corresponding to this approximate center of the selected loop(s) when the capsid fusion peptides assemble to form a virus or virus like particle. This includes the retention of the reading frame for that portion of the peptide sequence of the capsid that is synthesized downstream from the peptide insertion site.

In another embodiment, the influenza peptide can be inserted at the amino terminus of the capsid. The influenza peptide can be linked to the capsid through one or more linker sequences. In yet another embodiment, the influenza peptide can be inserted at the carboxy terminus of the capsid. The influenza peptide can also be linked to the carboxy terminus through one or more linkers, which can be cleavable by chemical or enzymatic hydrolysis. In one embodiment, the influenza peptide sequences are linked at both the amino and carboxy termini, or at one terminus and at least one internal location, such as a location that is expressed on the surface of the capsid in its three dimensional conformation. In one embodiment, at least one influenza antigenic peptide is expressed within at least one internal loop, or in at least one external surface loop, when the capsid fusion peptides are assembled to form a virus like particle.

More than one loop of the viral capsid can be modified. Embodiments of the present invention include wherein the influenza antigenic peptide is exposed on at least two surface loops when assembled as a virus or virus like particle. In another embodiment, at least two influenza antigenic peptides are inserted into a capsid protein and exposed on at least two surface loops of the viral capsid, cage, virus, or virus like particle. In another embodiment, at least three influenza antigenic peptides are inserted into the capsid protein and exposed on at least three surface loops of the virus or virus like particle. The influenza peptides in the surface loops can have the same amino acid sequence. In separate embodiments, the amino acid sequence of the influenza peptides in the surface loops can differ.

The nucleic acid sequence encoding the viral capsid protein can also be modified to alter the formation of a virus of virus like particle (see e.g. Brumfield, et al. (2004) J. Gen. Virol. 85: 1049-1053). For example, three general classes of modification are most typically generated for modifying virus or virus like particle assembly. These modifications are designed to alter the interior, exterior or the interface between adjacent subunits in the assembled protein cage. To accomplish this, mutagenic primers can be used to: (i) alter the interior surface charge of the viral nucleic acid binding region by replacing basic residues (e.g. K, R) in the N terminus with acidic glutamic acids (Douglas et al., 2002b); (ii) delete interior residues from the N terminus (for example, in CCMV, usually residues 4-37); (iii) insert a cDNA encoding an 11 amino acid peptide cell-targeting sequence (Graf et al., 1987) into a surface exposed loop; and (iv) modify interactions between viral subunits by altering the metal binding sites (for example, in CCMV, residues 81/148 mutant).

In one embodiment, the influenza antigenic peptide can be inserted into the capsid from a Cowpea Chlorotic Mottle Virus (CCMV). Embodiments of the present invention include wherein the influenza peptide can be inserted at amino acid 129 of the CCMV capsid protein in Seq ID. No. 11. In another embodiment, the influenza peptide sequence can be inserted at amino acids 60, 61, 62 or 63 of the CCMV capsid protein in SEQ ID No: 11. In still another embodiment, the influenza peptide can be inserted at amino acids 129 and amino acids 60-63 of the CCMV capsid protein in SEQ ID No: 11. In one embodiment, an M2 peptide selected from the group consisting of SEQ ID Nos: 3, 22, 23, and 24, or derivative or homologue thereof is inserted into the CCMV capsid protein.

In one embodiment, the influenza antigenic peptide can be inserted into the small capsid from a Cowpea Mosaic Virus (CPMV). Embodiments of the present invention include wherein the influenza peptide can be inserted between amino acid 22 and 23 of the CPMV small capsid protein (S CPMV Capsid) in SEQ ID No: 12. In one embodiment, an M2 peptide selected from the group consisting of SEQ ID Nos: 3, 22, 23, and 24, or derivative or homologue thereof is inserted into the CPMV small capsid protein.

In one embodiment, the influenza antigenic peptide can be inserted into the large capsid from a Cowpea Mosaic Virus (CPMV). Embodiments of the present invention include wherein the influenza peptide can be inserted into CPMV large capsid protein (L CPMV) in SEQ ID No: 13. In one embodiment, an M2 peptide selected from the group consisting of SEQ ID Nos: 3, 22, 23, and 24 or derivative or homologue thereof is inserted into the CPMV large capsid protein.

In one embodiment, a tag sequence adjacent to the influenza antigenic peptide of interest, or linked to a portion of the viral capsid protein, can also be included. In one embodiment, this tag sequence allows for purification of the recombinant capsid fusion peptide. The tag sequence can be an affinity tag, such as a hexa-histidine affinity tag. In another embodiment, the affinity tag can be a glutathione-S-transferase molecule. The tag can also be a fluorescent molecule, such as YFP or GFP, or analogs of such fluorescent proteins. The tag can also be a portion of an antibody molecule, or a known antigen or ligand for a known binding partner useful for purification.

The present invention contemplates the use of synthetic or any type of biological expression system to produce the recombinant capsid peptides containing the influenza peptide. Current methods of capsid protein expression include insect cell expression systems, bacterial cell expression systems such as E. coli, B. subtilus, and P. fluorescens, plant and plant cell culture expression systems, yeast expression systems such as S. cervisiae and P. Pastoris, and mammalian expression systems.

In one embodiment, a nucleic acid construct encoding a capsid fusion peptide is expressed in a host cell selected from a plant cell, including whole plants and plant cell cultures, or a Pseudomonas fluorescens cell. In one embodiment, a nucleic acid construct encoding the capsid fusion peptide is expressed in a whole plant host. In other embodiments, a nucleic acid construct encoding the capsid fusion peptide is expressed in a plant cell culture. In still another embodiment, a nucleic acid construct encoding the capsid fusion peptide is expressed in a Pseudomonas fluorescens. Techniques for expressing capsid fusion peptides in the above host cells are described in, for example, U.S. Pat. No. 5,874,087, U.S. Pat. No. 5,958,422, U.S. Pat. No. 6,110,466. U.S. application Ser. No. 11/001,626, and U.S. application Ser. No. 11/069,601 as well as in the Examples below.

d. Assembly of Virus or Virus Like Particles

The capsid fusion peptides of the present invention can be purified from a host cell and assembled in vitro to form virus like particles or cage structures, wherein the virus like particle does not contain host cell plasma membrane. Once the recombinant capsid fusion peptide is expressed in a host cell, it can be isolated and purified to substantial purity by standard techniques well known in the art. The isolation and purification techniques can depend on the host cell utilized to produce the capsid fusion peptides. Such techniques can include, but are not limited to, PEG, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, selective precipitation with such substances as column chromatography, immunopurification methods, size exclusion chromatography, immunopurification methods, centrifugation, ultracentrifugation, density gradient centrifugation (for example, on a sucrose or on a cesium chloride (CsCl) gradient), ultrafiltration through a size exclusion filter, and any other protein isolation methods known in the art. For example, capsid protein fusion peptide having established molecular adhesion properties can be reversibly fused to a ligand. With the appropriate ligand, the capsid protein fusion peptide can be selectively adsorbed to a purification column and then freed from the column in a relatively pure form. The capsid protein is then removed by enzymatic activity. In addition, the capsid protein fusion peptide can be purified using immunoaffinity columns or Ni-NTA columns. General techniques are further described in, for example, R. Scopes, Peptide Purification Principles and Practice, Springer-Verlag: N.Y. (1982); Deutscher, Guide to Peptide Purification, Academic Press (1990); U.S. Pat. No. 4,511,503; S. Roe, Peptide Purification Techniques: A Practical Approach (Practical Approach Series), Oxford Press (2001); D. Bollag, et al., Peptide Methods, Wiley-Lisa, Inc. (1996); A K Patra et al., Peptide Expr Purif, 18(2): p/182-92 (2000); and R. Mukhija, et al., Gene 165(2): p. 303-6 (1995). See also, for example, Ausubel, et al. (1987 and periodic supplements); Deutscher (1990) “Guide to Peptide Purification,” Methods in Enzymology vol. 182, and other volumes in this series; Coligan, et al. (1996 and periodic Supplements) Current Protocols in Peptide Science Wiley/Greene, NY; and manufacturer's literature on use of peptide purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, for example, Hochuli (1989) Chemische Industrie 12:69-70; Hochuli (1990) “Purification of Recombinant Peptides with Metal Chelate Absorbent” in Setlow (ed.) Genetic Engineering, Principle and Methods 12:87-98, Plenum Press, NY; and Crowe, et al. (1992) QIAexpress: The High Level Expression & Peptide Purification System QIAGEN, Inc., Chatsworth, Calif.

In other embodiments, the capsid fusion peptides expressed in host cells, especially bacterial host cells, may form insoluble aggregates (“inclusion bodies”). Several protocols are suitable for purification of peptides from inclusion bodies. For example, purification of inclusion bodies typically involves the extraction, separation and/or purification of inclusion bodies by disruption of the host cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension is typically lysed using 2-3 passages through a French Press. The cell suspension can also be homogenized using a Polytron (Brinknan Instruments) or sonicated on ice. Alternate methods of lysing bacteria are apparent to those of skill in the art (see, e.g., Sambrook et al., supra; Ausubel et al., supra).

If necessary, the inclusion bodies can be solubilized, and the lysed cell suspension typically can be centrifuged to remove unwanted insoluble matter. Capsid fusion peptides that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Although guanidine hydrochloride and similar agents are denaturants, this denaturation is not irreversible and renaturation may occur upon removal (by dialysis, for example) or dilution of the denaturant. Other suitable buffers are known to those skilled in the art.

Alternatively, it is possible to purify the recombinant capsid fusion peptides, virus like particles, or cage structures from the host periplasm. After lysis of the host cell, when the recombinant peptide is exported into the periplasm of the host cell, the periplasmic fraction of the bacteria can be isolated by cold osmotic shock in addition to other methods known to those skilled in the art. To isolate recombinant peptides from the periplasm, for example, the bacterial cells can be centrifuged to form a pellet. The pellet can be resuspended in a buffer containing 20% sucrose. To lyse the cells, the bacteria can be centrifuged and the pellet can be resuspended in ice-cold 5 mM MgSO₄ and kept in an ice bath for approximately 10 minutes. The cell suspension can be centrifuged and the supernatant decanted and saved. The recombinant peptides present in the supernatant can be separated from the host peptides by standard separation techniques well known to those of skill in the art.

An initial salt fractionation can separate many of the unwanted host cell peptides (or peptides derived from the cell culture media) from the recombinant capsid protein fusion peptides of interest. One such example can be ammonium sulfate. Ammonium sulfate precipitates peptides by effectively reducing the amount of water in the peptide mixture. Peptides then precipitate on the basis of their solubility. The more hydrophobic a peptide is, the more likely it is to precipitate at lower ammonium sulfate concentrations. A typical protocol includes adding saturated ammonium sulfate to a peptide solution so that the resultant ammonium sulfate concentration is between 20-30%. This concentration will precipitate the most hydrophobic of peptides. The precipitate is then discarded (unless the peptide of interest is hydrophobic) and ammonium sulfate is added to the supernatant to a concentration known to precipitate the capsid protein fusion peptide of interest. The precipitate is then solubilized in buffer and the excess salt removed if necessary, either through dialysis or diafiltration. Other methods that rely on solubility of peptides, such as cold ethanol precipitation, are well known to those of skill in the art and can be used to fractionate complex capsid protein fusion peptide mixtures.

The molecular weight of a recombinant capsid protein fusion peptide can be used to isolate it from peptides of greater and lesser size using ultrafiltration through membranes of different pore size (for example, Amicon or Millipore membranes). As a first step, the capsid protein fusion peptide mixture can be ultrafiltered through a membrane with a pore size that has a lower molecular weight cut-off than the molecular weight of the recombinant capsid fusion peptide of interest. The retentate of the ultrafiltration can then be ultrafiltered against a membrane with a molecular cut off greater than the molecular weight of the capsid protein fusion peptide of interest. The recombinant capsid protein fusion peptide will pass through the membrane into the filtrate. The filtrate can then be chromatographed as described below. Recombinant capsid fusion peptides can also be separated from other peptides on the basis of its size, net surface charge, hydrophobicity, and affinity for ligands. In addition, antibodies raised against the capsid proteins can be conjugated to column matrices and the capsid proteins immunopurified. All of these methods are well known in the art. It will be apparent to one of skill that chromatographic techniques can be performed at any scale and using equipment from many different manufacturers (e.g., Pharmacia Biotech).

Virus like particle assembly requires correctly folded capsid proteins. However, additional factors significant for VLP formulation and stability may exist, including pH, ionic strength, di-sulfide bonds, divalent cation bonding, among others. See, for example, Brady et al, (1977) “Dissociation of polyoma virus by the chelation of calcium ions found associated with purified virions,” J. Virol. 23(3):717-724; Gajardo et al, (1997) “Two proline residues are essential in the calcium binding activity of rotavirus VP7 outer capsid protein,” J. Virol., 71:2211-2216; Walter et al, (1975) “Intermolecular disulfide bonds: an important structural feature of the polyoma virus capsid,” Cold Spring Har. Symp. Quant. Biol., 39:255-257 (1975); Christansen et al, (1977) “Characterization of components released by alkali disruption of simian virus 40,” J Virol., 21:1079-1084; Salunke et al, (1986) “Self-assembly of purified polyomavirus capsid protein VP1,” Cell 46:895-904; Salunke et al, (1989) “Polymorphism in the assembly of polyomavirus capsid protein VP,” Biophys. J., 56:887-900; Garcea et al, (1983) “Host range transforming gene of polyoma virus plays a role in virus assembly,” Proc. Natl. Acad. Sci. USA, 80:3613-3617; Xi et al, (1991) “Baculovirus expression of the human papillomavirus type 16 capsid proteins: detection of L1-L2 protein complexes,” J. Gen. Virol., 72:2981-2988. Techniques that may be utilized for the re-assembly are well known in the art, and include, but are not limited to, techniques as described in the Example 6.

In addition, the capsid fusion peptides of the present invention can be expressed in a host cell, and assembled in vivo as virus, virus like particles, or cage structures, wherein the virus or virus like particle does not contain host cell plasma membrane. In one embodiment, a virus, virus like particle (VLP), or cage structure is formed in the host cell during or after expression of the capsid fusion peptide. In one embodiment, the virus, virus like particle, or cage exposes the influenza peptide on the surface of the virus or virus like particle.

In one embodiment, the virus, virus like particle, or cage structure is assembled as a multimeric assembly of recombinant capsid fusion peptides, including from three to about 200 capsid fusion peptides. In one embodiment, the virus, virus like particle, or cage structure includes at least 30, at least 50, at least 60, at least 90 or at least 120 capsid fusion peptides. In another embodiment, each virus, virus like particle, or cage structure includes at least 150 capsid fusion peptides, at least 160, at least 170, or at least 180 capsid fusion peptides.

In one embodiment, the virus or virus like particle is assembled as an icosahedral structure. In another embodiment, the virus like particle or virus is assembled in the same geometry as the native virus that the capsid sequence is derived of. In a separate embodiment, however, the virus or virus like particle does not have the identical geometry of the native virus. In other embodiments, for example, the structure is assembled in a particle formed of multiple capsids fusion peptides but not forming a native-type virus particle. For example, a cage structure of as few as 3 viral capsids can be formed. In separate embodiments, cage structures of about 6, 9, 12, 15, 18, 21, 24, 27, 30, 33, 36, 39, 42, 45, 48, 51, 54, 57, or 60 capsids can be formed.

Purification of plant viruses or plant virus particles assembled in vivo has been previously described. For example, see Dijkstra, J. and De Jager, C. P., 1998; Matthews, R. E. F., 1991, Plant Virology, Third Edition, Academic Press, Inc., Harcourt Brace Jovanovich, Publishers, and the Examples below. Most viruses can be isolated by a combination of two or more of the following procedures: high speed sedimentation, density gradient fractionation, precipitation using polyethylene glycol, salt precipitation, gel filtration, chromatography, and dialysis. Once virus or virus like particle containing cells are broken and the cell contents released and mixed, the virus or virus like particles find themselves in an environment that is abnormal. Therefore, it is often necessary to use an artificial medium designed to preserve the virus or virus like particles in an intact and unaggregated state during the various stages of isolation. The conditions that favor stability of purified virus or virus like particle preparations may be different from those needed in crude extracts or partially purified preparations. Moreover, different factors may interact strongly in the extent to which they affect virus stability. The main factors to be considered in developing a suitable medium are: pH and buffer system, metal ions and ionic strength, reducing agents and substances protecting against phenolic compounds, additives that remove plant proteins and ribosomes, enzymes, and detergents.

Many viruses are stable over a rather narrow pH range, and the extract must be maintained within this range. Choice of buffer may be important. Phosphate buffers have often been employed, but these may have deleterious effects on some virus or virus like particles. Some virus or virus like particles require the presence of divalent metal ions for the preservation of structural integrity. Ionic strength may be also important. Reducing agents are frequently added to the extraction media. These materials assist in preservation of virus or virus like particles that readily lose infectivity through oxidation. They may also reduce adsorption of host constituents to the virus. Phenolic materials may cause serious difficulties in the isolation and preservation of virus or virus like particles. Several methods have been used more or less successfully to minimize the effect of phenols on plant virus or virus like particles during isolation. EDTA as the sodium salt at 0.01 M in pH 7.4 buffer causes the disruption of most ribosomes, preventing their co-sedimentation with the virus particles. This substance can be used for viruses that do not require divalent metal ions for stability. Ribonucleases, ribosomes, 19 S protein, and green particulate material from fragmented chloroplasts can readily be absorbed by bentonite under certain magnesium concentration. Charcoal may be used to absorb and remove host materials, particularly pigments. Enzymes can be added to the initial extract for various purposes. For example, pectinase and cellulase aids in the release of the virus or virus like particles that would otherwise remain in the fiber fraction. The enzymes also digest materials that would otherwise co-precipitate with the virus or virus like particles. Triton X-100 or Tween 80 can sometimes be used in the initial extraction medium to assist in release of virus or virus like particles from insoluble cell components. Detergents may also assist in the initial clarification of the plant extract. Nonionic detergents dissociate cellular membranes, which may contaminate virus or virus like particles.

A variety of procedures can be used to crush or homogenize the virus or virus like particle containing plant tissue. These include (i) a pestle and mortar, (ii) various batch-type food blenders and juice extractors, and (iii) roller mills, colloid mills, and commercial meat mincers, which can cope with kilograms of tissue. If an extraction medium is used, it is often necessary to ensure immediate contact of broken cells with the medium. The homogenized tissue is usually pressed through cheesecloth to separate virus containing plant sap and crushed plant tissue. In the crude extract, the virus or virus like particles are mixed with a variety of cell constituents that are in the same broad size range as the virus or virus like particle and that may have properties that are similar in some respects. These particles include ribosomes, 19 S protein from chloroplasts, which has a tendency to aggregate, phytoferritin, membrane fragments, and fragments of broken chloroplasts. Also present are unbroken cells, all the smaller soluble proteins of the cell, and low molecular weight solutes. The first step in virus isolation is usually designed to remove as much of the macromolecular host material as possible, leaving the virus or virus like particles in solution. The extraction medium may be designed to precipitate ribosomes and other high molecular weight host materials or to disintegrate them. The extract may be subject to such treatment as heating, organic solvents such as chloroform or n-butanol-chloroform. The treated extract is then subjected to centrifugation at fairly low speed. This treatment sediments cell debris and coagulated host material. Centrifugation at high speed for a sufficient time will sediment the virus or virus like particles. This is a very useful step, as it serves the double purpose of concentrating the virus particles and removing low molecular weight materials. Certain plant viruses are preferentially precipitated in a single phase polyethylene glycol (PEG) system, although some host DNA may also be precipitated. Precipitation with PEG is one of the most common procedures used in virus or virus like particle isolation. The exact conditions for precipitation depend on pH, ionic strength, and concentration of macromolecules. Its application to the isolation of any particular virus is empirical. The main advantage of PEG precipitation is that expensive ultracentrifuges are not required, although differential centrifugation is often used as a second step in purification procedures. Many viruses may form pellets that are very difficult to re-suspend. Density gradient centrifugation offers the possibility of concentrating such virus or virus like particles without pelleting and is used in the isolation procedure for many viruses. A centrifuge tube is partially filled with a solution having a decreasing density from the bottom to the top of the tube. For plant viruses, sucrose is commonly used to form the gradient, and the virus solution is layered on top of the gradient. With gradients formed with cesium salts, the virus or virus like particles may be distributed throughout the solution at the start of the sedimentation or they may be layered on top of the density gradient. Density gradients may be used in three ways: (i) isopycnic gradient centrifugation, (ii) rate zonal sedimentation, and (iii) equilibrium zonal sedimentation. Following centrifugation, virus bands may be visualized due to their light scattering properties. Salt precipitation is also commonly employed. Ammonium sulfate at concentrations up to about one-third saturation is most commonly used, although many other salts will precipitate virus or virus like particles. After standing for some hours or days the virus or virus like particles are centrifuged down at low speed and re-dissolved in a small volume of a suitable medium. Many proteins have low solubility at or near their isoelectric points. Isoelectric precipitation can be used for virus or virus like particles that are stable under the conditions involved. The precipitate is collected by centrifugation or filtration and is re-dissolved in a suitable medium. Dialysis through cellulose membranes can be used to remove low molecular weight materials from an initial extract and to change the medium. It is more usually employed to remove salt following salt precipitation or crystallization, or following density gradient fractionation in salt or sucrose solutions.

Virus or virus like particle preparations taken through one step of purification and concentration will still contain some low and high molecular weight host materials. More of these can be removed by further purification steps. The procedure depends on the stability of the virus or virus like particle and the scale of the preparation. Sometimes highly purified preparations can be obtained by repeated application of the same procedure. For example, a preparation may be subjected to repeated PEG precipitations, or may be given several cycles of high and low speed sedimentation. The latter procedure leads to the preferential removal of host macromolecules because they remain insoluble when the pellets from a high speed sedimentation are resuspended. Generally speaking, during an isolation it is useful to apply at least two procedures that depend on different properties of the virus or virus like particles. This is likely to be more effective in removing host constituents than repeated application of the same procedure. One of the most useful procedures for further purification, particularly of less stable virus or virus like particles, is density gradient centrifugation. Sucrose is the most commonly used material for making the gradient. Sucrose density gradient centrifugation is frequently the method of choice for further purification. Strong solutions of salts such as cesium chloride are also effective gradient materials for viruses that are sufficiently stable. Successive fractionation in two different gradients may sometimes give useful results. Filtration through agar gel or Sephadex may offer a useful step for the further purification of virus or virus like particles that are unstable to the pelleting involved in the high speed centrifugation. Monoclonal antiviral antibodies can be bound to a support matrix such as agarose to form a column that will specifically bind the virus from a solution passed through the column. Virus can be eluted by lowering the pH. Chromatographic procedures can be used to give an effective purification step for partially purified preparations. For example, a column of calcium phosphate gel in phosphate buffer, cellulose column, or fast protein liquid chromatography can be used to purify various viruses.

At various stages in the isolation of a virus, it is necessary to concentrate virus and remove salts or sucrose. High speed centrifugation is commonly employed for the concentration of virus and the reduction of the amount of low molecular weight material. Dialysis is used for removal or exchange of salts.

### II. Antigenic Influenza Whole Protein or Protein Fragments

The present invention utilizes, in combination with the above described capsid fusion peptides containing an influenza peptide, at least one isolated antigenic protein or protein fragment, derivative, or homologue thereof, derived from an influenza virus, including a human and/or avian influenza virus. In one embodiment, the isolated antigenic protein or protein fragment, derivative, or homologue thereof, is derived from a newly emergent influenza viral strain.

The influenza viral protein or protein fragment utilized in the present invention can be a protein or protein fragment derived from the M1, M2, HA, NA, NP, PB1, PB2, PA or NP2 proteins, derivative, or homologue thereof, of an identified influenza viral strain. A large number of influenza strains, and corresponding protein sequences, have been identified and the sequences are publicly available through the National Center for Biotechnology Information (NCBI) Influenza Virus Resource site, available at http://www.ncbi.nlm.nih.gov/genomes/FLU/FLU.html.

In one embodiment of the present invention, the protein or protein fragment derived from an influenza virus is selected from the group consisting of an HA and NA proteins or protein fragments. Additional embodiments of the present invention include the NA protein or protein fragment is derived from the group of influenza NA proteins selected from the group consisting of subtypes N1, N2, N3, N4, N5, N6, N7, N8, and N9. In one embodiment, the influenza viral peptide is a protein or protein fragment derived from a human and/or avian influenza NA protein.

In other embodiments, the influenza viral antigenic protein or protein fragment is derived from an influenza HA protein. Additional embodiments of the present invention include the HA protein or protein fragment is derived from the group of influenza HA proteins selected from the group consisting of the subtypes H1, H2, H3, H4, H5, H6, H7, H8, H9, H10, H11, H12, H13, H14, and H15. Embodiments of the present invention include wherein the HA peptide is derived from the group of human and/or avian influenza HA proteins. In a additional embodiments the HA peptide can be derived from an avian influenza HA protein. In one embodiment, the avian HA protein is selected from the subtypes H5, H7, and H9.

In one embodiment of the present invention, the isolated antigenic protein or protein fragment is selected from a newly emergent strain of influenza. The World Health Organization reviews the world influenza epidemiological data twice annually, and updates periodically the identification of newly emergent strains of influenza. Genetic information useful in deriving isolated antigenic proteins or protein fragments for use in the present invention is available to those of skill in the art. For example, the Los Alamos National Laboratory maintains an Influenza Sequence Database available at http://www-flu.lanl.gov/ which contains genetic information on newly emergent strains of influenza.

Embodiments of the present invention also include wherein the HA protein or protein fragment combined with the virus like particle is derived from the 568 amino acid sequence of the A/Thailand/3(SP-83)/2004(H5N1) strain in SEQ ID No: 15 (Table 5), derivative, or homologue thereof, that is encoded by the nucleotide sequence SEQ ID No: 16 (Table 5). In other embodiments, the influenza virus protein utilized in the present invention can be the entire amino acid sequence of the HA protein or protein fragment of the A/Thailand/3(SP-83)/2004(H5N1) strain, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 540, 560, 565, 568 or more amino acids selected from SEQ ID No: 15. The influenza virus protein selected can be at least 75, 80, 85, 90, 95, 98, or 99% homologous to the amino acid sequence of the HA protein or protein fragment of the A/Thailand/3(SP-83)/2004(H5N1) strain, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 540, 560, 565, 568 or more amino acids selected from SEQ ID No: 15, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, or more amino acids selected from SEQ ID No: 15. In other embodiments, the influenza protein or nucleic acid sequence can be altered to improve the characteristics of the protein, such as, but not limited to, improved expression in the host, enhanced immunogenicity, or improved covalent binding properties.

Alternatively, the HA protein or protein fragment combined with the virus like particle is derived from SEQ ID No: 17 (Table 5). In other embodiments, the influenza virus protein utilized in the present invention can be the entire amino acid sequence of the HA protein or protein fragment of SEQ ID No: 17, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 530, 537, or more amino acids selected from SEQ ID No: 17. The influenza virus protein selected can be at least 75, 80, 85, 90, 95, 98, or 99% homologous to the amino acid sequence of SEQ ID No: 17, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 530, 537, or more amino acids selected from SEQ ID No: 17. In other embodiments, the influenza protein or nucleic acid sequence can be altered to improve the characteristics of the protein, such as, but not limited to, improved expression in the host, enhanced immunogenicity, or improved covalent binding properties.

In other embodiments the HA protein fragment will be the 36 kDa HA1 fragment of the A/Thailand/3(SP-83)/2004(H5N1) strain (SEQ ID No: 18, Table 5) encoded by the nucleotide sequence SEQ ID No: 19 (Table 5). In other embodiments, the influenza virus protein utilized in the present invention can be the entire amino acid sequence of the HA protein or protein fragment of SEQ ID No: 18, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 350, 352, or more amino acids selected from SEQ ID No: 18. The influenza virus protein selected can be at least 75, 80, 85, 90, 95, 98, or 99% homologous to the amino acid sequence of SEQ ID No: 18, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 350, 352, or more amino acids selected from SEQ ID No: 18. In other embodiments, the influenza protein or nucleic acid sequence can be altered to improve the characteristics of the protein, such as, but not limited to, improved expression in the host, enhanced immunogenicity, or improved covalent binding properties.

In another embodiment the HA protein fragment will be the 26 kDa HA2 fragment of the A/Thailand/3(SP-83)/2004(H5N1) strain (SEQ ID No: 20, Table 5) encoded by the nucleotide sequence SEQ ID No: 21 (Table 5). In other embodiments, the influenza virus protein utilized in the present invention can be the entire amino acid sequence of the HA protein or protein fragment of SEQ ID No: 20, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, or more amino acids selected from SEQ ID No: 20. The influenza virus protein selected can be at least 75, 80, 85, 90, 95, 98, or 99% homologous to the amino acid sequence of SEQ ID No: 20, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, or more amino acids selected from SEQ ID No: 20. In other embodiments, the influenza protein or nucleic acid sequence can be altered to improve the characteristics of the protein, such as, but not limited to, improved expression in the host, enhanced immunogenicity, or improved covalent binding properties.

In embodiments of the present invention, the HA protein or protein fragment combined with the virus like particle is derived from the 565 amino acid sequence of the A/Vietnam/CL20/2004(H5N1) strain in SEQ ID No: 25 (Table 5), derivative, or homologue thereof, that is encoded by the nucleotide sequences SEQ ID No: 26-28 (Table 5). In other embodiments, the influenza virus protein utilized in the present invention can be the entire amino acid sequence of the HA protein or protein fragment of the A/Vietnam/CL20/2004(H5N1) strain, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 540, 560, 565 or more amino acids selected from SEQ ID No: 25. In one embodiment the influenza virus protein utilized in the present invention can be the HA protein fragment of the A/Vietnam/CL20/2004(H5N1) strain in SEQ ID No: 29 (Table 5) that lacks the native N-terminal signal and C-terminal transmembrane domain and cytoplasmic tail. The influenza virus protein selected can be at least 75, 80, 85, 90, 95, 98, or 99% homologous to the amino acid sequence of the HA protein or protein fragment of the A/Vietnam/CL20/2004(H5N1) strain, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 520, 540, 560, 565 or more amino acids selected from SEQ ID No: 25 and SEQ ID No: 29, or a subset thereof comprising at least 20, 25, 30, 35, 40, 45, 50, 55, 60, 65, 70, 75, 80, 85, 90, 95, 100, 110, 120, 130, 140, 150, 160, 170, 180, 190, or more amino acids selected from SEQ ID No: 25 and SEQ ID No: 29. In other embodiments, the influenza protein or nucleic acid sequence can be altered to improve the characteristics of the protein, such as, but not limited to, improved expression in the host, enhanced immunogenicity, or improved covalent binding properties.

TABLE 5
HA Protein and Nucleic Acid Sequence

| | | |
|---|---|---|
| Sequence | Name | SEQ ID No: |

| | | |
|---|---|---|
| MEKIVLLFAIVSLVKSDQICIGYHANNSTEQVDTIMEKNV | HA-A/Thailand/3 | SEQ ID No: 15 |
| TVTHAQDILEKTHNGKLCDLDGVKPLILRDCSVAGWLLGN | (SP-83)/2004 | |
| PMCDEFINVPEWSYIVEKANPVNDLCYPGDFNDYEELKHL | (H5N1) | |
| LSRINHFEKIQIIPKSSWSSHEASLGVSSACPYQGKSSFF | | |
| RNVVWLIKKNSTYPTIKRSYNNTNQEDLLVLWGIHHPNDA | | |
| AEQTKLYQNPTTYISVGTSTLNQRLVPRIATRSKVNGQSG | | |
| RMEFFWTILKYNDAINFESNGNFIAYEYAYKIVKKGDSTI | | |
| MKSELEYGNCNTKCQTPMGAINSSMPFHNIHPLTIGECPK | | |
| YVKSNRLVLATGLRNSPQRERRRKKRGLFGAIAGFIEGGW | | |
| QGMVDGWYGYHHSNEQGSGYAADKESTQKAIDGVTNKVNS | | |
| IIDKMNTQFEAVGREFNNLERRIENLNKKMEDGFLDVWTY | | |
| NAELLVLMENERTLDFHDSNVKNLYDKVRLQLRDNAKELG | | |
| NGCFEFYHKCDNECMESVRNGTYDYPQYSEEARLKREEIS | | |
| GVKLESIGIYQILSIYSTVASSLALAIMVAGLSLWMCSNG | | |
| SLQCRICI | | |
| ATGGAGAAGATAGTTCTCTTGTTTGCCATCGTCAGTTTGG | Plant codon optimized | SEQ ID No: 16 |
| TCAAATCAGATCAGATTTGTATAGGATACCATGCAAACAA | nucleic acid sequence | |
| CAGTACCGAACAAGTTGACACAATCATGGAGAAGAATGTA | HA-A/Thailand/3 | |
| ACAGTGACTCACGCCCAGGACATTCTTGAGAAGACCCACA | (SP-83)/2004(H5N1) | |
| ATGGCAAGCTTTGCGACTTGGATGGTGTTAAGCCACTCAT | | |
| TCTTCGTGATTGTTCTGTGGCAGGTTGGCTTCTCGGAAAC | | |
| CCAATGTGTGACGAGTTCATCAACGTTCCAGAGTGGTCTT | | |
| ACATCGTCGAGAAGGCAAACCCTGTGAATGATGTTTGCTA | | |
| CCCAGGAGACTTCAACGACTACGAGGAATTGAAACATCTC | | |
| TTGTCTAGGATCAACCACTTTGAGAAGATTCAGATCATTC | | |
| CTAAGTCCTCTTGGTCTTCACATGAGGCAAGCCTTGGTGT | | |
| GTCATCCGCCTGCCCTTATCAAGGAAAGTCATCTTTCTTC | | |
| AGAAATGTTGTGTGGCTTATCAAGAAGAACTCTACATATC | | |
| CAACCATCAAGAGGAGCTACAACAACACAAACCAGGAAGA | | |
| TCTCTTGGTGCTCTGGGGAATTCATCATCCAAATGACGCA | | |
| GCAGAGCAAACTAAGCTTTACCAGAACCCTACAACTTACA | | |
| TCTCCGTGGGCACTTCTACACTCAATCAGAGACTTGTGCC | | |
| AAGGATTGCTACTAGGTCAAAGGTTAACGGACAATCAGGT | | |
| CGTATGGAGTTCTTCTGGACAATCTTGAAGCCAAACGATG | | |
| CCATCAACTTCGAGTCAAATGGAAACTTCATCGCTCCAGA | | |
| GTACGCTTACAAGATTGTGAAGAAAGGAGATAGTACCATC | | |
| ATGAAGTCTGAACTCGAGTACGGAAACTGCAACACCAAGT | | |
| GTCAGACTCCAATGGGAGCTATCAATAGCTCTATGCCATT | | |
| TCACAACATTCACCCTTTGACAATAGGAGAATGCCCTAAG | | |
| TACGTGAAGAGCAACAGGCTCGTCCTCGCAACTGGTTTGA | | |
| GAAACAGTCCACAAAGAGAACGTAGACGTAAGAAGAGAGG | | |
| ATTGTTCGGTGCAATTGCCGGGTTCATCGAAGGAGGCTGG | | |
| CAGGGTATGGTGGATGGTTGGTATGGGTATCATCACAGTA | | |
| ATGAGCAAGGATCAGGATATGCTGCAGACAAAGAAAGCAC | | |
| CCAGAAAGCAATAGATGGAGTCACTAACAAAGTCAATTCC | | |
| ATAATCGACAAGATGAACACACAGTTCGAAGCTGTTGGAC | | |
| GTGAGTTCAACAACCTTGAGAGGAGGATTGAGAATCTTAA | | |
| CAAGAAGATGGAAGATGGGTTCTTGGACGTGTGGACTTAC | | |
| AATGCTGAATTGTTAGTTCTTATGGAGAACGAAAGAACTC | | |
| TCGACTTCCATGATTCTAACGTGAAGAACTTGTACGACAA | | |
| GGTGCGTCTTCAACTTCGTGATAACGCTAAAGAGCTCGGG | | |
| AACGGTTGCTTTGAGTTCTATCACAAGTGTGACAATGAGT | | |
| GCATGGAATCTGTTAGAAATGGAACTTACGATTACCCTCA | | |
| GTATTCAGAGGAGGCAAGGCTCAAGAGAGAAGAGATCTCC | | |
| GGCGTGAAGTTGGAGAGCATTGGTATCTACCAACATCATC | | |
| ACCATCACCACTAA | | |
| MEKIVLLFAIVSLVKSDQICIGYHANNSTEQVDTIMEKNV | HA-A/Thailand/3 | SEQ ID No: 17 |
| TVTHAQDILEKTHNGKLCDLDGVKPLILRDCSVAGWLLGN | (SP-83)/2004(H5N1) | |
| PMCDEFINVPEWSYIVEKANPVNDLCYPGDFNDYEELKHL | | |
| LSRINHFEKIQIIPKSSWSSHEASLGVSSACPYQGKSSFF | | |
| RNVVWLIKKNSTYPTIKRSYNNTNQEDLLVLWGIHHPNDA | | |
| AEQTKLYQNPTTYISVGTSTLNQRLVPRIATRSKVNGQSG | | |
| RMEFFWTILKPNDAINFESNGNFIAPEYAYKIVKKGDSTI | | |
| MKSELEYGNCNTKCQTPMGAINSSMPFHNIHPLTIGECPK | | |
| YVKSNRLVLATGLRNSPQRERRRKKRGLFGAIAGFIEGGW | | |
| QGMVDGWYGYHHSNEQGSGYAADKESTQKAIDGVTNKVNS | | |
| IIDKMNTQFEAVGREFNNLERRIENLNKKMEDGFLDVWTY | | |
| NAELLVLMENERTLDFHDSNVKNLYDKVRLQLRDNAKELG | | |
| NGCFEFYHKGDNECMESVRNGTYDYPQYSEEARLKREEIS | | |
| GVKLESIGIYQHHHHHH | | |
| MEKIVLLFAIVSLVKSDQICIGYHANNSTEQVDTIMEKNV | HA1-A/Thailand/3 | SEQ ID No: 18 |
| TVTHAQDILEKTHNGKLCDLDGVKPLILRDCSVAGWLLGN | (SP-83)/2004(H5N1) | |
| PMCDEFINVPEWSYIVEKANPVNDLCYPGDFNDYEELKHL | | |
| LSRINHFEKIQIIPKSSWSSHEASLGVSSACPYQGKSSFF | | |
| RNVVWLIKKNSTYPTIKRSYNNTNQEDLLVLWGIHHPNDA | | |
| AEQTKLYQNPTTYISVGTSTLNQRLVPRIATRSKVNGQSG | | |
| RMEFFWTILKPNDAINFESNGNFIAPEYAYKIVKKGDSTI | | |
| MKSELEYGNCNTKCQTPMGAINSSMPFHNIHPLTIGECPK | | |
| YVKSNRLVLATGLRNSPQRERRRKKRHHHHHH | | |
| ATGGAGAAGATAGTTCTCTTGTTTGCCATCGTCAGTTTGG | Plant codon optimized | Seq. ID. No. 19 |
| TCAAATCAGATCAGATTTGTATAGGATACCATGCAAACAA | HA1-A/Thailand/3 | |
| CAGTACCGAACAAGTTGACACAATCATGGAGAAGAATGTA | (SP-83)/2004(H5N1) | |
| ACAGTGACTCACGCCCAGGACATTCTTGAGAAGACCCACA | | |
| ATGGCAAGCTTTGCGACTTGGATGGTGTTAAGCCACTCAT | | |
| TCTTCGTGATTGTTCTGTGGCAGGTTGGCTTCTCGGAAAC | | |
| CCAATGTGTGACGAGTTCATCAACGTTCCAGAGTGGTCTT | | |
| ACATCGTCGAGAAGGCAAACCCTGTGAATGATCTTTGCTA | | |
| CCCAGGAGACTTCAACGACTACGAGGAATTGAAACATCTC | | |
| TTGTCTAGGATCAACCACTTTGAGAAGATTCAGATGATTC | | |
| CTAAGTCCTCTTGGTCTTCACATGAGGCAAGCCTTGGTGT | | |
| GTCATCCGCCTGCCCTTATCAAGGAAAGTCATCTTTCTTC | | |
| AGAAATGTTGTGTGGCTTATCAAGAAGAACTCTACATATC | | |
| CAACCATCAAGAGGAGCTACAACAACACAAACCAGGAAGA | | |
| TCTCTTGGTGCTCTGGGGAATTCATCATCCAAATGACGCA | | |
| GCAGAGCAAACTAAGCTTTACCAGAACCCTACAACTTACA | | |
| TCTCCGTGGGCACTTCTACACTGAATCAGAGACTTGTGCC | | |
| AAGGATTGCTACTAGGTCAAAGGTTAACGGACAATCAGGT | | |
| CGTATGGAGTTCTTCTGGACAATCTTGAAGCCAAACGATG | | |
| CCATCAACTTCGAGTCAAATGGAAACTTCATCGCTCCAGA | | |
| GTACGCTTACAAGATTGTGAAGAAAGGAGATAGTACCATC | | |
| ATGAAGTCTGAACTCGAGTACGGAAACTGCAACACCAAGT | | |
| GTCAGACTCCAATGGGAGCTATCAATAGCTCTATGCCATT | | |
| TCACAACATTCACCCTTTGACAATAGGAGAATGCCCTAAG | | |
| TACGTGAAGAGCAACAGGCTCGTCCTCGCAACTGGTTTGA | | |
| GAAACAGTCCACAAAGAGAACGTAGACGTAAGAAGAGACA | | |
| TCATCACCATCACCACTAA | | |
| MEKIVLLFAIVSLVKSGLFGAIAGFIEGGWQGMVDGWYGY | HA2-A/Thailand/3 | Seq. ID. No. 20 |
| HHSNEQGSGYAADKESTQKAIDGVTNKVNSIIDKMNTQFE | (SP-83)/2004(H5N1) | |
| AVGREFNNLERRIENLNKKMEDGFLDVWTYNAELLVLMEN | | |
| ERTLDFHDSNVKNLYDKVRLQLRDNAKELGNGCFEFYHKC | | |
| DNECMESVRNGTYDYPQYSEEARLKREEISGVKLESIGIY | | |
| QHHHHHH | | |
| ATGGAGAAGATAGTTCTCTTGTTTGCCATCGTCAGTTTGG | Plant codon optimized | Seq. ID. No. 21 |
| TCAAATCAGGATTGTTCGGTGCAATTGCCGGGTTCATCGA | HA2-A/Thailand/3 | |
| AGGAGGCTGGCAGGGTATGGTGGATGGTTGGTATGGGTAT | (SP-83)/2004(H5N1) | |
| CATCACAGTAATGAGCAAGGATCAGGATATGCTGCAGACA | | |
| AAGAAAGCACCCAGAAAGCAATAGATGGAGTCACTAACAA | | |
| AGTCAATTCCATAATCGACAAGATGAACACACAGTTCGAA | | |
| GCTGTTGGACGTGAGTTCAACAACCTTGAGAGGAGGATTG | | |
| AGAATCTTAACAAGAAGATGGAAGATGGGTTCTTGGACGT | | |
| GTGGACTTACAATGCTGAATTGTTAGTTCTTATGGAGAAC | | |
| GAAAGAACTCTCGACTTCCATGATTCTAACGTGAAGAACT | | |
| TGTACGACAAGGTGCGTCTTCAACTTCGTGATAACGCTAA | | |
| AGAGCTCGGGAACGGTTGCTTTGAGTTCTATCACAAGTGT | | |
| GACAATGAGTGCATGGAATCTGTTAGAAATGGAACTTACG | | |
| ATTACCCTCAGTATTCAGAGGAGGCAAGGCTCAAGAGAGA | | |
| AGAGATCTCCGGCGTGAAGTTGGAGAGCATTGGTATCTAC | | |
| CAACATCATCACCATCACCACTAA | | |
| MEKIVLLFAIVSLVKSDQICIGYHANNSTEQVDTLMEKNV | HA-A/Vietnam | Seq. ID. No. 25 |
| TVTHAQDILEKTHNGKLCDLDGVKPLILRDCSVAGWLLGN | CL20/2004(H5N1) | |
| PMCDEFINVPEWSYIVEKANPVNDLCYPGDFDDYEELKHL | | |
| LSRINHFEKIQIIPKSSWSSHEASLGVSSACPYQGKSSFF | | |
| RNVVWLIKKNSTYPTIKRSYNNTNQEDLLVMWGIHHPNDA | | |
| AEQTKLYQNPTTYISVGTSTLNQRLVPRIATRSKVNGQSG | | |
| RMEFFWTILKPNDAINFESNGNFIAPEYAYKIVKKGDSTI | | |
| MKSELEYGNCNTKCQTPMGAINSSMPFHNIHPLTIGECPK | | |
| YVKSNRLVLATGLRNSPQRERRRKKRGLFGAIAGFIEGGW | | |
| QGMVDGWYGYHHSNEQGSGYAADKESTQKAIDGVTNKVNS | | |
| IIDKMNTQFEAVGREFNNLERRILENLNKKMEDGFLDVWT | | |
| YNAELLVLMENERTLDFHDSNVKNLYDKVRLQLRDNAKEL | | |
| GNGCFEFYHKCDNECMESVRNGTYDYPQYSEEARLKREEI | | |
| SGVKLESIGIYQILSIYSTVASSLALAIMVAGLSLWMCSN | | |
| GSLQCR | | |
| GGACTAGTAGGAGGTAACTTATGGAGAAAATCGTCCTGTT | Codon optimized | Seq. ID. No. 26 |
| GTTTGCCATTGTCTCCCTGGTGAAGAGCGACCAGATTTGC | HA-A/Vietnam/ | |
| ATCGGCTATCACGCGAACAATTCCACCGAACAAGTGGATA | CL20/2004(H5N1) | |
| CGATCATGGAGAAGAATGTGACCGTCACCCACGCTCAGGA | containing the SpeI and | |
| TATTCTGGAGAAGACGCATAACGGGAAACTCTGTGACTTG | XhoI restriction sites | |
| GATGGGGTTAAGCCGCTGATTCTGCGCGATTGTTCGGTGG | and a ribosome binding | |
| CCGGCTGGCTGCTGGGCAACCCAATGTGCGATGAATTTAT | site for expression in P. | |
| CAACGTGCCCGAGTGGAGCTACATTGTCGAGAAGGCCAAT | fluorescens | |
| CCCGTTAACGACTTGTGCTACCCTGGTGATTTCGACGACT | | |
| ACGAAGAACTGAAGCACCTGTTGTCCCGCATTAATCACTT | | |
| CGAGAAAATCCAGATCATCCCGAAATCGAGCTGGAGCAGC | | |
| CATGAAGCCTCGCTCGGTGTGAGTTCCGCCTGTCCGTACC | | |
| AGGGCAAGTCGTCCTTCTTCCGTAACGTGGTGTGGCTGAT | | |
| TAAGAAGAACTCCACTTACCCGACCATTAAGCGGAGCTAC | | |
| AACAACACCAACCAAGAAGACTTGTTGGTGATGTGGGGTA | | |
| TCCATCACCCCAACGACGCCGCCGAGCAAACCAAACTGTA | | |
| CCAGAATCCTACGACTTACATCTCGGTCGGCACCAGCACC | | |
| CTGAACCAACGCTTGGTTCCGCGCATCGCGACTCGCAGCA | | |
| AAGTCAACGGCCAGAGTGGGCGTATGGAATTCTTTTGGAC | | |
| CATCCTGAAGCCAAACGATGCGATCAACTTCGAATCGAAT | | |
| GGCAACTTCATTGCCCCGGAATACGCCTACAAGATCGTGA | | |
| AGAAAGGGGACTCGACCATCATGAAGTCGGAGCTGGAATA | | |
| CGGCAACTGCAACACGAAATGCCAGACGCCGATGGGCGCC | | |
| ATCAACTCCAGCATGCCGTTTCATAACATTCACCCATTGA | | |
| CTATCGGCGAATGCCCGAAATACGTCAAGTCCAATCGTCT | | |
| GGTCCTGGCGACCGGTCTGCGCAACAGCCCGCAGCGCGAA | | |
| CGTCGCCGTAAGAAACGGGGCCTGTTCGGTGCCATCGCTG | | |
| GCTTCATCGAGGGCGGCTGGCAGGGCATGGTCGACGGCTG | | |
| GTATGGCTACCATCACAGCAACGAGCAGGGCAGTGGTTAC | | |
| GCCGCTGACAAGGAAAGCACCCAAAAGGCCATCGACGGCG | | |
| TGACGAACAAGGTGAACTCCATTATCGACAAGATGAACAC | | |
| GCAGTTCGAAGCCGTCGGCCGTGAGTTCAACAACCTGGAA | | |
| CGCCGCATCGAAAACTTGAACAAGAAGATGGAAGACGGTT | | |
| TCTTGGACGTCTGGACCTATAATGCGGAATTGCTGGTTCT | | |
| GATGGAAAACGAACGCACCCTGGACTTTCATGACTCGAAC | | |
| GTGAAGAACCTGTATGATAAAGTCCGTCTGCAGCTGCGCG | | |
| ACAACGCCAAGGAACTGGGTAACGGCTGCTTTGAATTTTA | | |
| CCATAAATGTGACAATGAGTGCATGGAAAGTGTGCGCAAC | | |
| GGCACCTATGATTATCCGCAGTACAGTGAAGAGGCACGTC | | |
| TGAAGCGTGAGGAAATTAGCGGCGTTAAATTGGAGAGCAT | | |
| CGGGATCTATCAGATCCTCAGCATCTACAGCACCGTGGCC | | |
| AGCAGCTTGGCCCTGGCCATCATGGTCGCTGGCCTCTCGC | | |
| TGTGGATGTGCAGCAACGGTTCCCTGCAGTGCCGCTGATA | | |
| ATAGCTCGAGTT | | |
| GGACTAGTAGGAGGTAACTTATGGAAAAGATTGTGCTGTT | Codon optimized | Seq. ID. No. 27 |
| GTTCGCCATCGTGAGTCTGGTGAAATCGGACCAAATCTGC | HA-A/Vietnam/ | |
| ATCGGCTACCACGCTAATAACAGCACCGAACAAGTCGACA | CL20/2004(H5N1) | |
| CCATCATGGAGAAGAACGTCACTGTGACGCATGCCCAAGA | containing the SpeI and | |
| TATCTTGGAAAAGACCCATAACGGCAAGCTGTGCGACCTG | XhoI restriction sites | |
| GACGGTGTGAAGCCGTTGATCCTGCGCGACTGCTCCGTCG | and a ribosome binding | |
| CGGGTTGGCTGTTGGGCAACCCGATGTGCGATGAGTTCAT | site for expression in P. | |
| TAACGTCCCGGAATGGAGCTATATCGTCGAGAAGGCGAAT | fluorescens | |
| CCCGTCAACGACCTGTGTTACCCTGGCGATTTCGATGATT | | |
| ACGAAGAGCTGAAACATCTGCTGAGCCGCATCAACCACTT | | |
| CGAGAAGATCCAAATCATCCCGAAGAGCAGTTGGAGCAGC | | |
| CACGAAGCCTCCCTGGGCGTTTCGTCGGCCTGCCCCTATC | | |
| AGGGGAAGTCGTCCTTTTTCCGCAACGTGGTCTGGCTGAT | | |
| CAAAAAGAAGAGTACCTATCCTACTATCAAGCGCAGTTAC | | |
| AACAACACTAACCAAGAAGACCTGTTGGTCATGTGGGGCA | | |
| TTCATCATCCCAACGACGCGGCCGAGCAGACCAAGTTGTA | | |
| CCAGAACCCGACCACGTATATCAGCGTGGGGACGTCCACC | | |
| CTCAATCAGCGTCTGGTGCCGCGCATCGCGACCCGTAGCA | | |
| AGGTGAACGGGCAGTCGGGCCGGATGGAGTTCTTTTGGAC | | |
| TATCCTGAAGCCGAACGACGCAATCAACTTCGAGTCGAAT | | |
| GGTAACTTCATTGCCCCAGAGTATGCTTACAAGATCGTGA | | |
| AAAAGGGCGACTCGACTATCATGAAGAGCGAACTGGAGTA | | |
| CGGGAACTGTAACACCAAATGTCAAACCCCGATGGGCGCA | | |
| ATCAACAGCTCGATGCCCTTCCATAATATCCATCCGCTGA | | |
| CCATTGGTGAGTGCCCGAAGTACGTCAAATCGAACCGGTT | | |
| GGTGCTGGCCACTGGCCTCCGTAACTCGCCGCAGCGGGAA | | |
| CGTCGCCGTAAGAAACGCGGTTTGTTCGGCGCCATTGCAG | | |
| GGTTCATCGAGGGCGGCTGGCAGGGCATGGTCGATGGTTG | | |
| GTACGGGTACCACCACTCCAACGAACAAGGCAGCGGCTAC | | |
| GCGGCGGATAAAGAAAGTACCCAGAAGGCTATCGACGGCG | | |
| TCACCAACAAAGTGAACAGCATCATCGATAAGATGAACAC | | |
| GCAGTTCGAAGCCGTGGGCCGTGAGTTCAACAACCTCGAA | | |
| CGGCGCATCGAGAACCTGAACAAAAAGATGGAAGATGGCT | | |
| TCCTGGATGTCTGGACCTATAATGCCGAGCTGCTGGTGCT | | |
| GATGGAAAACGAGCGTACCCTGGACTTTCACGATTCGAAT | | |
| GTGAAGAATCTGTACGACAAAGTCCGGTTGCAGCTGCGCG | | |
| ACAACGCGAAAGAGCTGGGCAACGGCTGTTTCGAGTTCTA | | |
| CCATAAGTGCGACAACGAGTGTATGGAGTCCGTGCGCAAC | | |
| GGCACGTATGATTATCCTCAGTATTCCGAAGAGGCCCGCT | | |
| TGAAACGTGAAGAAATCAGCGGCGTGAAGCTGGAGAGCAT | | |
| CGGCATCTATCAAATCTTGAGCATCTATAGCACCGTGGCG | | |
| TCGTCGCTGGCCCTCGCGATCATGGTTGCCGGCCTGAGCC | | |
| TGTGGATGTGCAGCAACGGCTGGCTGCAATGCCGCTGATA | | |
| ATAGCTCGAGTT | | |
| GGACTAGTAGGAGGTAACTTATGGAGAAAATCGTCCTGTT | Codon optimized | Seq. ID. No. 28 |
| GTTTGCCATTGTCTCCCTGGTGAAGAGCGACCAGATTTGC | HA-A/Vietnam/ | |
| ATCGGCTATCACGCGAACAATTCCACCGAACAAGTGGATA | CL20/2004(H5N1) | |
| CGATCATGGAGAAGAATGTGACCGTCACCCACGCTCAGGA | containing the SpeI and | |
| TATTCTGGAGAAGACGCATAACGGGAAACTCTGTGACTTG | XhoI restriction sites | |
| GATGGGGTTAAGCCGCTGATTCTGCGCGATTGTTCGGTGG | and a ribosome binding | |
| CCGGCTGGCTGCTGGGCAACCCAATGTGCGATGAATTTAT | site for expression in P. | |
| CAACGTGCCCGAGTGGAGCTACATTGTCGAGAAGGCCAAT | fluorescens | |
| CCCGTTAACGACTTGTGCTACCCTGGTGATTTCGACGACT | | |
| ACGAAGAACTGAAGCACCTGTTGTCCCGCATTAATCACTT | | |
| CGAGAAAATCCAGATCATCCCGAAATCGAGCTGGAGCAGC | | |
| CATGAAGCCTCGCTCGGTGTGAGTTCCGCCTGTCCGTACC | | |
| AGGGCAAGTCGTCCTTCTTCCGTAACGTGGTGTGGCTGAT | | |
| TAAGAAGAACTCCACTTACCCGACCATTAAGCGGAGCTAC | | |
| AACAACACCAACCAAGAAGACTTGTTGGTGATGTGGGGTA | | |
| TCCATCACCCCAACGACGCCGCCGAGCAAACCAAACTGTA | | |
| CCAGAATCCTACGACTTACATCTCGGTCGGCACCAGCACC | | |
| CTGAACCAACGCTTGGTTCCGCGCATCGCGACTCGCAGCA | | |
| AAGTCAACGGCCAGAGTGGGCGTATGGAATTCTTTTGGAC | | |
| CATCCTGAAGCCAAACGATGCGATCAACTTCGAATCGAAT | | |
| GGCAAGTTCATTGCCCCGGAATACGCCTACAAGATCGTGA | | |
| AGAAAGGGGACTCGACCATCATGAAGTCGGAGCTGGAATA | | |
| CGGCAACTGCAACACGAAATGCCAGACGCCGATGGGCGCC | | |
| ATCAACTCCAGCATGCCGTTTCATAACATTCACCCATTGA | | |
| CTATCGGCGAATGCCCGAAATACGTCAAGTCCAATCGTCT | | |
| GGTCCTGGCGACCGGTCTGCGCAACAGCCCGCAGCGCGAA | | |
| CGTCGCCGTAAGAAACGGGGCCTGTTCGGTGCCATCGCTG | | |
| GCTTCATCGAGGGCGGCTGGCAGGGCATGGTCGACGGCTG | | |
| GTATGGCTACCATCACAGCAACGAGCAGGGCAGTGGTTAC | | |
| GCCGCTGACAAGGAAAGCACCCAAAAGGCCATCGACGGCG | | |
| TGACGAACAAGGTGAACTCCATTATCGACAAGATGAACAC | | |
| GCAGTTCGAAGCCGTCGGCCGTGAGTTCAACAACCTGGAA | | |
| CGCCGCATCGAAAACTTGAACAAGAAGATGGAAGACGGTT | | |
| TCTTGGACGTCTGGACCTATAATGCGGAATTGCTGGTTCT | | |
| GATGGAAAACGAACGCACCCTGGACTTTCATGACTCGAAC | | |
| GTGAAGAACCTGTATGATAAAGTCCGTCTGCAGCTGCGCG | | |
| ACAACGCCAAGGAACTGGGTAACGGCTGCTTTGAATTTTA | | |
| CCATAAATGTGACAATGAGTGCATGGAAAGTGTGCGCAAC | | |
| GGCACCTATGATTATCCGCAGTACAGTGAAGAGGCACGTC | | |
| TGAAGCGTGAGGAAATTAGCGGCGTTAAATTGGAGAGCAT | | |
| CGGGATCTATCAGATCCTCAGCATCTACAGCACCGTGGCC | | |
| AGCAGCTTGGCCCTGGCCATCATGGTCGCTGGCCTCTCGC | | |
| TGTGGATGTGCAGCAACGGTTCCCTGCAGTGCCGCTGATA | | |
| ATAGCTCGAGTA | | |
| DQICIGYHANNSTEQVDTIMEKNVTVTHAQDILEKTHNGK | HA-A/Vietnam/ | Seq. ID. No. 29 |
| LCDLDGVKPLILRDCSVAGWLLGNPMCDEFINVPEWSYIV | CL20/2004(H5N1) | |
| EKANPVNDLCYPGDFDDYEELKHLLSRINHFEKIQIIPKS | fragment | |
| SWSSHEASLGVSSACPYQGKSSFFRNVVWLIKKNSTYPTI | | |
| KRSYNNTNQEDLLVMWGIHHPNDAAEQTKLYQNPTTYISV | | |
| GTSTLNQRLVPRIATRSKVNGQSGRMEFFWTILKLPNDAI | | |
| NFESNGNFIAPEYAYKIVKKGDSTIMKSELEYGNCNTKCQ | | |
| TPMGAINSSMPFHNIHPLTIGECPKYVKSNRLVLATGLRN | | |
| SPQRERRRKKRGLFGAIAGFIEGGWQGMVDGWYGYHHSNE | | |
| QGSGYAADKESTQKAIDGVTNKVNSIIDKMNTQFEAVGRE | | |
| FNNLERRIENLNKKMEDGFLDVWTYNAELLVLMENERTLD | | |
| FHDSNVKNLYDKVRLQLRDNAKELGNGCFEFYHKCDNECM | | |
| ESVRNGTYDYPQYSEEARLKREEISGVKLESIGIYQ | | |

In one embodiment, the virus like particle containing the influenza peptide is combined with at least one NA protein or protein fragment derived from an influenza virus, including a human or avian influenza virus, and at least one HA protein or protein fragment derived from an influenza virus, including a human or avian influenza virus. In an additional embodiment, the virus like particle containing the influenza peptide is combined with at least one NA protein or protein fragment derived from an influenza virus, at least one HA protein or protein fragment derived from an influenza virus, and any combination of influenza viral proteins or protein fragments, including human and/or avian influenza proteins or protein fragments, selected from the group consisting of M1, M2, NP, PB1, PB2, PA, and NP2, derivative or homolog thereof.

a. Production of Antigenic Proteins or Protein Fragments

The present invention contemplates the use of synthetic or any type of biological expression system to produce the influenza antigenic proteins or protein fragments. Current methods of protein expression include insect cell expression systems, bacterial cell expression systems such as E. coli, B. subtilus, and P. fluorescens, plant and plant cell culture expression systems, yeast expression systems such as S. cervisiae and P. pastoris, and mammalian expression systems.

In one embodiment, the protein or protein fragment is expressed in a host cell selected from a plant cell, including whole plants and plant cell cultures, or a Pseudomonas fluorescens cell. Additional embodiments of the present invention include the protein or protein fragment is expressed in a whole plant host. In additional embodiments the protein or protein fragment is expressed in a plant cell culture. Techniques for expressing recombinant protein or protein fragments in the above host cells are well known in the art. In one embodiment plant viral vectors are used to express influenza proteins or protein fragments in whole plants or plant cells. Embodiments of the present invention include wherein PVX vector is used to express HA proteins or protein fragments in Nicotiana benthamiana plants In another embodiment PVX vector is used to express HA proteins or protein fragments in tobacco NT1 plant cells. Techniques for utilizing viral vectors are described in, for example, U.S. Pat. No. 4,885,248, U.S. Pat. No. 5,173,410, U.S. Pat. No. 5,500,360, U.S. Pat. No. 5,602,242, U.S. Pat. No. 5,804,439, U.S. Pat. No. 5,627,060, U.S. Pat. No. 5,466,788, U.S. Pat. No. 5,670,353, U.S. Pat. No. 5,633,447, and U.S. Pat. No. 5,846,795, as well as in the Examples 14 and 15 below. In other embodiments, transgenic plants or plant cell cultures are used to express HA proteins or protein fragments. Methods utilized for expression of proteins or protein fragments in transgenic plants or plant cells are well known in the art. In other embodiments and in Example 18 and Example 19 the HA proteins or protein fragments are expressed in the cytoplasm or periplasm of Pseudomonas fluorescens.

Methods that can be utilized for the isolation and purification of the influenza protein or protein fragment expressed in a host cell are similar to, or the same as, those previously described in the examples for capsid fusion peptide isolation and purification.

### III. Combination of Influenza Peptide Containing VLPs and Influenza Antigenic Proteins

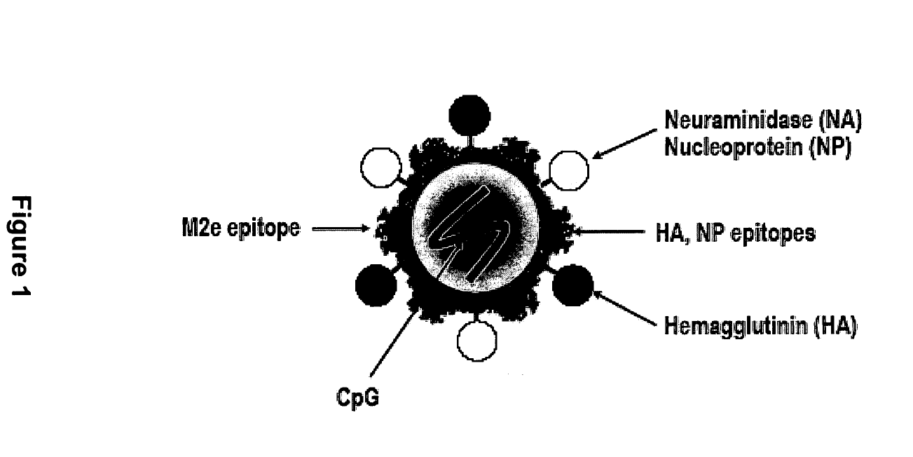

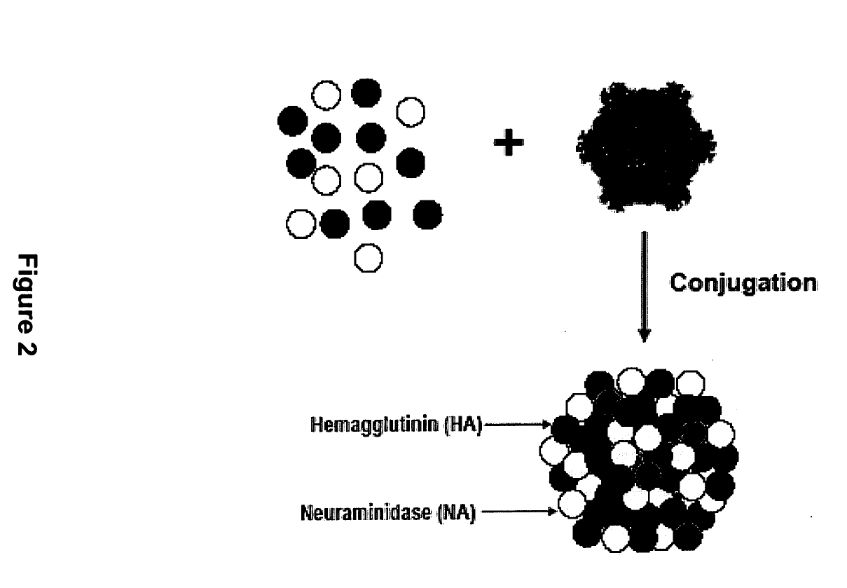

The present invention provides compositions for use as vaccines against the influenza virus comprising i) at least one peptide derived from an influenza virus, wherein the peptide is fused to a capsid protein derived from a plant virus forming a recombinant capsid fusion peptide, and wherein the recombinant capsid fusion peptide is capable of assembly to form a virus or virus like particle, and ii) at least one antigenic protein or protein fragment derived from an influenza virus. In one embodiment of the present invention, the antigenic protein or protein fragments are not chemically attached or linked to the virus like particles. In other embodiments, the antigenic influenza proteins or protein fragments are chemically conjugated to the virus or virus like particle. See, for example, FIGS. 1 and 2.

The antigenic influenza proteins or protein fragments and the virus like particles of the present invention can be conjugated using any conjugation method in the art. See for example Gillitzer E, Willits D, Young M, Douglas T. (2002) “Chemical modification of a viral cage for multivalent presentation,” Chem Commun (Camb) 20:2390-1; Wang Q, Kaltgrad E, Lin T, Johnson J E, Finn M G (2002) “Natural supramolecular building blocks. Wild-type cowpea mosaic virus,” Chem Biol. 9(7):805-11; Wang Q, Lin T, Tang L, Johnson J E, Finn M G. (2002) “Icosahedral virus particles as addressable nanoscale building blocks,” Angew Chem Int Ed Engl. 41(3):459-62; Raja et al. (2003) “Hybrid virus-polymer materials. 1. Synthesis and properties of Peg-decorated cowpea mosaic virus,” Biomacromolecules 4:472-476; Wang Q, Lin T, Johnson J E, Finn M G. (2002) “Natural supramolecular building blocks. Cysteine-added mutants of cowpea mosaic virus,” Chem Biol. 9(7):813-9.

Other methods for conjugating may include, for example, using sulfosuccinimidyl 4-(N-maleimidomethyl)cyclohexane-1-carboxylate (sSMCC), N-[ε-maleimidocaproyloxy]sulfosuccinimide ester (sEMCS), N-maleimidobenzoyl-N-hydroxysuccinimide ester (MBS), glutaraldehyde, 1-ethyl-3-(3 dimethylaminopropyl)carbodiimide (EDCI), Bis-diazobenzidine (BDB), or N-acetyl homocysteine thiolactone (NAHT).

In the carrier maleimide-activation method, the conjugation is achieved using sulfosuccinimidyl 4-(N-maleimidomethyl)cyclohexane-1-carboxylate (sSMCC), or N-maleimidobenzoyl-N-hydroxysuccinimide ester (MBS). The method using sSMCC is widely used and highly specific (See, e.g., Meyer et al. 2002, J. of Virol. 76, 2150-2158). sSMCC cross-links the SH-group of a cysteine residue to the amino group of a lysine residue on the virus like protein.

In the conjugation reaction using sSMCC, the virus like particle is first activated by binding the sSMCC reagent to the amine (e.g.: lysine) residues of the virus or virus like particle. After separation of the activated virus or virus like particle from the excess reagent and the by-product, the cysteine-containing peptide is added and the link takes place by addition of the SH-group to the maleimide function of the activated virus or virus like particle. The method using MBS conjugates the peptide and the virus or virus like particle through a similar mechanism.

The conjugation using sSMCC can be highly specific for SH-groups. Thus, cysteine residues in the antigenic influenza protein or protein fragment are essential for facile conjugation. If an antigenic protein or protein fragment does not have a cysteine residue, a cysteine residue can be added to the peptide, preferably at the N-terminus or C-terminus. If the desired epitope in the protein or protein fragment contains a cysteine, the conjugation should be achieved with a method not using a sSMCC activated virus or virus like particle. If the protein or protein fragment contains more than one cysteine residue, the protein or protein fragment should not be conjugated to the virus or virus like particle using sSMCC unless the excess cysteine residue can be replaced or modified.

The linkage should not interfere with the desired epitope in the protein or protein fragment. The cysteine is preferably separated from the desired epitope sequence with a distance of at least one amino acid as a spacer.

Another conjugation useful in the present invention is achieved using N-acetyl homocysteine thiolactone (NAHT). For example, thiolactones can be used to introduce a thiol functionality onto the virus or virus like particle to allow conjugation with maleimidated or Bromo-acetylated-peptides (Tolman et al. Int. J. Peptide Protein Res. 41, 1993, 455-466; Conley et al. Vaccine 1994, 12, 445-451).

In additional embodiments of the invention, conjugation reactions to couple the protein or protein fragment to the virus or virus like particle involve introducing and/or using intrinsic nucleophilic groups on one reactant and introducing and/or using intrinsic electrophilic groups in the other reactant. One activation scheme would be to introduce a nucleophilic thiol group to the virus or virus like particle and adding electrophilic groups (preferably alkyl halides or maleimide) to the influenza protein or protein fragment. The resulting conjugate will have thiol ether bonds linking the protein or protein fragment and the virus or virus like particle. Direct reaction of the influenza protein or protein fragment's electrophilic group (maleimide or alkyl halide) and intrinsic nucleophilic groups (preferably primary amines or thiols) of the virus or virus like particle, leading to secondary amine linkages or thiol ether bonds. Alternative schemes involve adding a maleimide group or alkyl halide to the virus or virus like particle and introducing a terminal cysteine to the influenza protein or protein fragment and/or using intrinsic influenza protein thiols again resulting in thiol ether linkages.

A sulfur containing amino acid contains a reactive sulfur group. Examples of sulfur containing amino acids include cysteine and non-protein amino acids such as homocysteine. Additionally, the reactive sulfur may exist in a disulfide form prior to activation and reaction with the virus or virus like particle. For example, cysteines present in the influenza proteins or protein fragments can be used in coupling reactions to a virus or virus like particle activated with electrophilic groups such as maleimide or alkyl halides. Introduction of maleimide groups using heterobifunctional cross-linkers containing reactive maleimide and activated esters is common.

A covalent linker joining an influenza protein to a virus like particle may be stable under physiological conditions. Examples of such linkers are nonspecific cross-linking agents, monogenetic spacers and bigeneric spacers. Non-specific cross-linking agents and their use are well known in the art. Examples of such reagents and their use include reaction with glutaraldehyde; reaction with N ethyl-N′-(3-dimethylaminopropyl) carbodiimide, with or without admixture of a succinylated virus or virus like particles; periodate oxidation of glycosylated substituents followed by coupling to free amino groups of a virus or virus like particle in the presence of sodium borohydride or sodium cyanoborohydride; periodate oxidation of non-acylated terminal serine and threonine residues can create terminal aldehydes which can then be reacted with amines or hydrazides creating Schiff base or hydrazones which can be reduced with cyanoborohydride to secondary amines; diazotization of; aromatic amino groups followed by coupling on tyrosine side chain residues of the protein; reaction with isocyanates; or reaction of mixed anhydrides. See, generally, Briand, et al., 1985 J. Imm. Meth. 78:59.

Monogeneric spacers and their use are well known in the art. Monogeneric spacers are bifunctional and require functionalization of only one of the partners of the reaction pair before conjugation takes place. Bigeneric spacers and their use are well known in the art. Bigeneric spacers are formed after each partner of the reaction pair is functionalized. Conjugation occurs when each functionalized partner is reacted with its opposite partner to form a stable covalent bond or bonds. (See, for example, Marburg, et al., 1986 J. Am. Chem. Sot. 108:5282-5287, and Marburg, et al., U.S. Pat. No. 4,695,624).

An advantage of the present invention is that one can achieve various molar ratios of influenza protein to virus or virus like particle in the conjugate. This ‘peptide coupling load’ on virus or virus like particles can be varied by altering aspects of the conjugation procedure in a trial and error manner to achieve a conjugate having the desired properties. For example, if a high coupling load is desired such that every reactive site on the virus or virus like particle is conjugated to an influenza protein or protein fragment, one can assess the reactive sites on the virus or virus like particle and include a large molar excess of influenza protein or protein fragment in the coupling reaction. If a low density coupling load is desired, one can include a molar ratio of less than 1 mol influenza protein per mole of reactive sites on the virus or virus like particle.

The particular conditions one chooses will ultimately be guided by the yields achieved, physical properties of the conjugate, the potency of the resulting conjugate, the patient population and the desired dosage one wishes to administer. If the total protein in the vaccine is not an important consideration, one could formulate doses of conjugates of differing coupling loads and different immunogenicities to deliver the same effective dose. However, if total protein or volume is an important consideration, for example, if the conjugate is meant to be used in a combination vaccine, one may be mindful of the total volume or protein contributed by the conjugate to the final combination vaccine. One could then assess the immunogenicity of several conjugates having differing coupling loads and thereafter choose to use a conjugate with adequate immunogenicity and a level of total protein or volume acceptable to add to the combination vaccine.

### IV. Vaccines

The present invention provides compositions for use as vaccines against the influenza virus. In one embodiment, pharmaceutical compositions comprising compositions of the present invention can be prepared as acidic or basic salts. Pharmaceutically acceptable salts (in the form of water- or oil-soluble or dispersible products) include conventional non-toxic salts or the customary ammonium salts that are formed, e.g., from inorganic or organic acids or bases. Examples of such salts include acid addition salts such as acetate, adipate, alginate, aspartate, benzoate, benzenesulfonate, butyrate, citrate, camphorate, camphorsulfonate, cyclopentanepropionate, digluconate, dodecylsulfate, ethanesulfonate, fumarate, glucoheptanoate, glycerophosphate, hernisulfate, heptanoate, hexanoate, hydrochloride, hydrobromide hydroiodide, 2-hydroxyethanesulfonate, lactates maleate, methanesulfonate, 2-naphthalenesulfonate, nicotinate, oxalate pamoate, pectinate, persulfate, 3-phenylpropionate, picrate, pivalate, propionate, succinate tartrate, thioeyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

In one embodiment of the present invention, the compositions of the present invention are administered to an animal or patient without an adjuvant. In other embodiments, the compositions are administered with an adjuvant.

Aluminum based adjuvants are commonly used in the art and include Aluminum phosphate, Aluminum hydroxide, Aluminum hydroxy-phosphate and aluminum hydroxy-sulfate-phosphate. Trade names of adjuvants in common use include ADJUPHOS, MERCK ALUM and ALHYDROGEL. The composition can be bound to or co-precipitated with the adjuvant as desired and as appropriate for the particular adjuvant used.

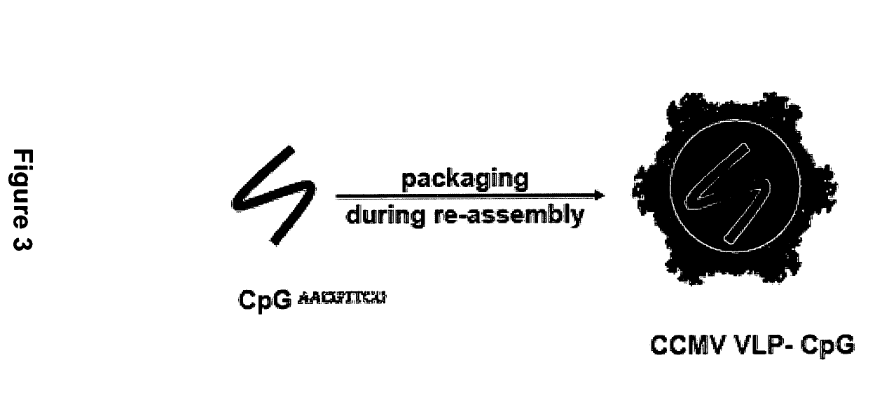

Non-aluminum adjuvants can also be used. Non-aluminum adjuvants include QS21, Lipid-A and derivatives or variants thereof, Freund's complete or incomplete adjuvant, neutral liposomes, liposomes containing vaccine and cytokines or chemokines. Additional adjuvants include immuno-stimulatory nucleic acids, including CpG sequences. See, for example, FIG. 3.

The compositions of the present invention can be administered using any technique currently utilized in the art, including, for example, orally, mucosally, intravenously, intramuscularly, intrathecally, epidurally, intraperitoneally or subcutaneously. Embodiments of the present invention include wherein the composition is delivered mucosally through the nose, mouth, or skin. Additional embodiments of the present invention include the composition is delivered intranasally. In other embodiments, the composition is administered orally by digesting a plant host cell the composition was produced in. In another embodiment, the composition is administered transdermally via a patch.

Suitable dosing regimens are preferably determined taking into account factors well known in the art including age, weight, sex and medical condition of the subject; the route of administration; the desired effect; and the particular composition employed (e.g., the influenza protein, the protein loading on the virus or virus like particle, etc.). The vaccine can be used in multi-dose vaccination formats.

In one embodiment, a dose would consist of the range from about 1 ug to about 1.0 mg total protein. In another embodiment of the present invention the range is from about 0.01 mg to 1.0 mg. However, one may prefer to adjust dosage based on the amount of peptide delivered. In either embodiment, these ranges are guidelines. More precise dosages can be determined by assessing the immunogenicity of the conjugate produced so that an immunologically effective dose is delivered. An immunologically effective dose is one that stimulates the immune system of the patient to establish an immunological response. Preferably, the level of immune system stimulation will be sufficient to develop an immunological memory sufficient to provide long term protection against disease caused by infection with a particular influenza virus.

The timing of doses depends upon factors well known in the art. After the initial administration one or more booster doses may subsequently be administered to maintain antibody titers. An example of a dosing regime would be a dose on day 1, a second dose at 1 or 2 months, a third dose at either 3, 4, 5, 6, 7, 8, 9, 10, 11, or 12 months or greater than 12 months, and additional booster doses at distant times as needed.

The immune response so generated can be completely or partially protective against disease and debilitating symptoms caused by infection with influenza virus.

### VI. Methods for Producing a Combination of Influenza Peptide Containing VLPs and Influenza Antigenic Proteins

In another aspect of the present invention, a method of producing a composition for use in an influenza vaccine in a human or animal is provided comprising:
i) providing a first nucleic acid encoding a recombinant capsid fusion peptide comprising a plant virus capsid protein genetically fused to an influenza viral peptide selected from the group consisting of M1, M2, HA, NA, NP, PB1, PB2, PA and NP2, and expressing the first nucleic acid in a host cell, wherein the host cell is selected from a plant cell or Pseudomonas fluorescens cell;
ii) assembling the capsid fusion peptides to form a virus or virus like particle, wherein the virus or virus like particle does not contain plasma membrane or cell wall proteins from the host cell;
iii) providing at least one second nucleic acid encoding at least one antigenic protein or protein fragment derived from a newly emergent influenza virus strain, and expressing the second nucleic acid in a host cell, wherein the host cell is a plant cell or Pseudomonas fluorescens cell, and optionally wherein the newly emergent influenza virus strain is identified by the World Health Organization; and
iv) isolating and purifying the antigenic protein or protein fragment; and
v) combining the virus or virus like particle and the antigenic protein or protein fragment to form a composition capable of administration to a human or animal.

In one embodiment, the virus or virus like particle is produced in a plant host, for example, in whole plants or plant cell cultures. In other embodiments, the virus like particle is produced in a Pseudomonas fluorescens host cell. In one embodiment, the antigenic protein or protein fragment is produced in a plant host, for example, in whole plants or plant cell cultures. In other embodiments, the antigenic protein or protein fragment is produced in a Pseudomonas fluorescens host cell. In one embodiment, the virus or virus like particle and the antigenic protein or protein fragment are co-produced in the same plant or Pseudomonas fluorescens host cell, and the capsid fusion peptide assembles in vivo to form a virus or virus like particle. Alternatively, the antigenic protein and virus like particles are produced in a plant and/or Pseudomonas fluorescens host cell, isolated, and purified, wherein the capsid fusion peptide is assembled in vivo or re-assembled in vitro to form a virus like particle and combined with an influenza antigenic protein or protein fragment to form a composition capable of administration to a human or animal.

### EXAMPLES

### Example 1

### Cloning of the M2-e Universal Epitope of Influenza A Virus into Cowpea Chlorotic Mottle Virus (CCMV) Coat Protein (CP)

Two 23 AA peptides derived from an M2 protein of Influenza A virus: M2e-1 and M2e-2 were independently cloned into CCMV CP gene to be expressed on CCMV virus-like particles (VLPs).

| | |
|---|---|
| M2e-1 peptide sequence: | |
| SLLTEVETPIRNEWGCRCNDSSD | (Seq. ID. No. 1) |
| M2e-2 peptide sequence: | |
| SLLTEVETPIRNEWECRCNGSSD | (Seq. ID. No. 2) |

Each of the inserts was synthesized by over-lapping DNA oligonucleotides with the thermocycling program detailed below:

PCR PROTOCOL
Reaction Mix (100 μL total volume)

| | | |
|---|---|---|
| 10 | μL | 10X PT HIFI buffer * |
| 4 | μL | 50 mM MgSO₄ * |
| 2 | μL | 10 mM dNTPs * |
| 0.25 | ng | Each Primer |
| 1-5 | ng | Template DNA |
| 1 | μL | PT HIFI Taq DNA Polymerase * |

| | |
|---|---|
| Remainder | Distilled De-ionized H₂O (ddH₂O) |

Thermocycling Steps

| | | | | |
|---|---|---|---|---|
| Step 1 | 1 Cycle | 2 | min. | 94° C. |
| Step 2 | 35 Cycles | 30 | sec. | 94° C. |
| | | 30 | sec. | 55° C. |
| | | 1 | min. | 68° C. |
| Step 3 | 1 Cycle | 10 | min. | 70° C. |

| | | | |
|---|---|---|---|
| Step 4 | 1 Cycle | Maintain | 4° C. |

* (from Invitrogen Corp, Carlsbad, CA, USA, hereinafter “Invitrogen”)

The oligonucleotides utilized include:

M2e-1F

(Seq. ID. No. 14)

5′CGG GGA TCC TGT CAC TCT TGA CAG AGG TAG AAA CAC
CGA TAC GTA ATG AAT GG3′
M2e-1R

(Seq. ID. No. 30)

5′CGC AGG ATC CCA TCT GAA GAA TCA TTA CAA CGA CAG
CCC CAT TCA TTA CGT ATC3′
M2e-2F

(Seq. ID. No. 31)

5′CGG GGA TCC TGT CAC TCT TGA CAG AGG TAG AAA CAC
CGA TAC GTA ATG AAT GG3′
M2e-2R

(Seq. ID. No. 32)

5′CGC AGG ATC CCA TCT GAA GAG CCA TTA CAA CGA CAT
TCC CAT TCA TTA CG3′

Resulting PCR products were digested with BamHI restriction enzyme and subcloned into shuttle vector pESC-CCMV129 cut with BamHI and then dephosphorylated. The coding sequences of chimeric CCMV-CP genes were then sequenced to ensure the orientation of the inserted peptide sequence and the integrity of the modified CP gene. The chimeric coat protein genes were then excised out of the shuttle plasmid at SpeI and XhoI and subcloned into Pseudomonas fluorescens expression plasmid pDOW1803 at SpeI and XhoI. The resulting plasmids were then transformed by electroporation into electro-competent P. fluorescens MB214 with Tetracycline 15 ug/ml as the selection agent.

### Example 2

### Cloning of the NP Epitopes of Influenza A Virus into Cowpea Chlorotic Mottle Virus (CCMV) Coat Protein (CP)

Two peptides derived from an NP protein of Influenza A virus: NP55-69 and NP147-158 were independently cloned into CCMV CP gene to be expressed on CCMV virus-like particles (VLPs).

| | |
|---|---|
| NP55-69 peptide sequence: | |
| RLIQNSLTIERMVLS | (Seq. ID. No.9) |
| NP147-158 peptide sequence: | |
| TYQRTRALVRTG | (Seq. ID. No. 10) |

Each of the inserts was synthesized by over-lapping DNA oligonucleotides with the thermocycling program as detailed in Example 1.

The oligonucleotides include:

NP55-69F

(Seq. ID. No. 33)

5′GATCCTGCGCCTGATCCAGAACAGCCTGACCATCGAACGCATGGTGCT
GAGCGG3′
NP55-69R

(Seq. ID. No. 34)

5′GATCCCGCTCAGCACCATGCGTTCGATGGTCAGGCTGTTCTGGATCAG
GCGCAG3′
NP147-158F

(Seq. ID. No. 35)

5′GATCCTGACCTACCAGCGCACCCGCGCTCTGGTGCGCACCGGCGG3′
NP147-158R

(Seq. ID. No. 36)

5′GATCCCGCCGGTGCGCACCAGAGCGCGGGTGCGCTGGTAGGTCAG3′

Resulting PCR products were digested with BamHI restriction enzyme and subcloned into shuttle vector pESC-CCMV129 cut with BamHI and then dephosphorylated. The coding sequences of chimeric CCMV-CP genes were then sequenced to ensure the orientation of the inserted peptide sequence and the integrity of the modified CP gene. The chimeric coat protein genes were then excised out of the shuttle plasmid at SpeI and XhoI and subcloned into Pseudomonas fluorescens expression plasmid pDOW1803 at SpeI and XhoI. The resulting plasmids were then transformed by electroporation into electro-competent P. fluorescens MB214 with Tetracycline 15 ug/ml as the selection agent.

### Example 3

### Cloning of the HA Epitope of Influenza A Virus into Cowpea Chlorotic Mottle Virus (CCMV) Coat Protein (CP)

A peptide derived from an HA protein of Influenza A virus, HA 91-108 was independently cloned into CCMV CP gene to be expressed on CCMV virus-like particles (VLPs).

| | |
|---|---|
| HA91-108 peptide sequence: | |
| SKAFSNCYPYDVPDYASL | (Seq. ID. No. 7) |

The inserts was synthesized by over-lapping DNA oligonucleotides with the thermocycling program as detailed in the Example 1.

The oligonucleotides included:

HA91-108F

(Seq. ID. No. 37)

5′GATCCTGAGCAAGGCTTTCAGCAACTGCTACCCGTACGACGTGCCGGA
CTACGCTAGCCTGGG3′
HA91-108R

(Seq. ID. No. 38)

5′GATCCCCAGGCTAGCGTAGTCCGGCACGTCGTACGGGTAGCAGTTGCT
GAAAGCCTTGCTCAG3′

Resulting PCR products were digested with BamHI restriction enzyme and subcloned into shuttle vector pESC-CCMV129 cut with BamHI and then dephosphorylated. The coding sequences of chimeric CCMV-CP genes were then sequenced to ensure the orientation of the inserted peptide sequence and the integrity of the modified CP gene. The chimeric coat protein genes were then excised out of the shuttle plasmid at SpeI and XhoI and subcloned into Pseudomonas fluorescens expression plasmid pDOW1803 at SpeI and XhoI. The resulting plasmids were then transformed by electroporation into electro-competent P. fluorescens MB214 with Tetracycline 15 ug/ml as the selection agent.

### Example 4

### Expression of Recombinant CCMV Capsid Fusion Peptides

The CCMV129-fusion peptide expression plasmids were transformed into Pseudomonas fluorescens MB214 host cells according to the following protocol. Host cells were thawed gradually in vials maintained on ice. For each transformation, 1 μL purified expression plasmid DNA was added to the host cells and the resulting mixture was swirled gently with a pipette tip to mix, and then incubated on ice for 30 min. The mixture was transferred to electroporation disposable cuvettes (BioRad Gene Pulser Cuvette, 0.2 cm electrode gap, cat no. 165-2086). The cuvettes were placed into a Biorad Gene Pulser pre-set at 200 Ohms, 25 μfarads, 2.25 kV. Cells were pulse cells briefly (about 1-2 sec). Cold LB medium was then immediately added and the resulting suspension was incubated at 30° C. for 2 hours. Cells were then plated on LB tet15 (tetracycline-supplemented LB medium) agar and grown at 30° C. overnight.

One colony was picked from each plate and the picked sample was inoculated into 50 mL LB seed culture in a baffled shake flask. Liquid suspension cultures were grown overnight at 30° C. with 250 rpm shaking. 10 mL of each resulting seed culture was then used to inoculate 200 mL of shake-flask medium (i.e. yeast extracts and salt with trace elements, sodium citrate, and glycerol, pH 6.8) in a 1 liter baffled shake flask. Tetracycline was added for selection. Inoculated cultures were grown overnight at 30° C. with 250 rpm shaking and induced with IPTG for expression of the CCMV129-fusion peptide chimeric coat proteins.

1 mL aliquots from each shake-flask culture were then centrifuged to pellet the cells. Cell pellets were resuspended in 0.75 mL cold 50 mM Tris-HCl, pH 8.2, containing 2 mM EDTA. 0.1% volume of 10% TritonX-100 detergent was then added, followed by an addition of lysozyme to 0.2 mg/mL final concentration. Cells were then incubated on ice for 2 hours, at which time a clear and viscous cell lysate should be apparent.

To the lysates, 1/200 volume 1M MgCl2 was added, followed by an addition of 1/200 volume 2 mg/mL DNase I, and then incubation on ice for 1 hour, by which time the lysate should have become a much less viscous liquid. Treated lysates were then spun for 30 min at 4° C. at maximum speed in a tabletop centrifuge and the supernatants were decanted into clean tubes. The decanted supernatants are the “soluble” protein fractions. The remaining pellets were then resuspended in 0.75 mL TE buffer (10 mM Tris-Cl, pH 7.5, 1 mM EDTA). The resuspended pellets are the “insoluble” fractions.

### Example 5

### Analysis of Recombinant CCMV Capsid Fusion Peptides

The “soluble” and “insoluble” fractions were electrophoresed on NuPAGE 4-12% Bis-Tris gels (from Invitrogen, Cat. NP0323), having 1.0 mm×15 wells, according to manufacturer's specification. 5 ul of each fraction were combined with 5 ul of 2× reducing SDS-PAGE loading buffer, and boiled for 5 minutes prior to running on the gel. The gels were stained with SimplyBlue Safe Stain, (from Invitrogen, Cat. LC6060) and destained overnight with water.

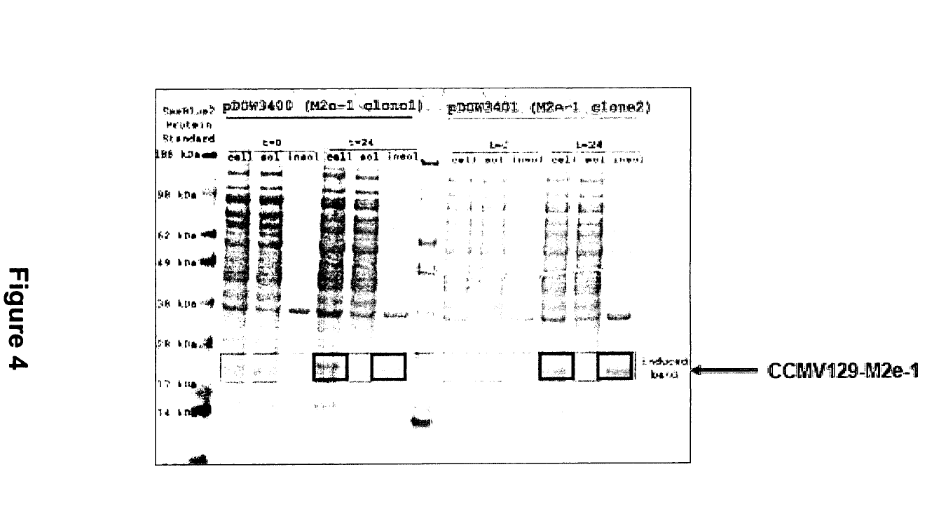

FIG. 4 shows expression of CCMV129 CP fused with M2e-1 influenza virus peptide in Pseudomonas fluorescens as detected by SDS-PAGE stained by Simply blue safe stain (Invitrogen).

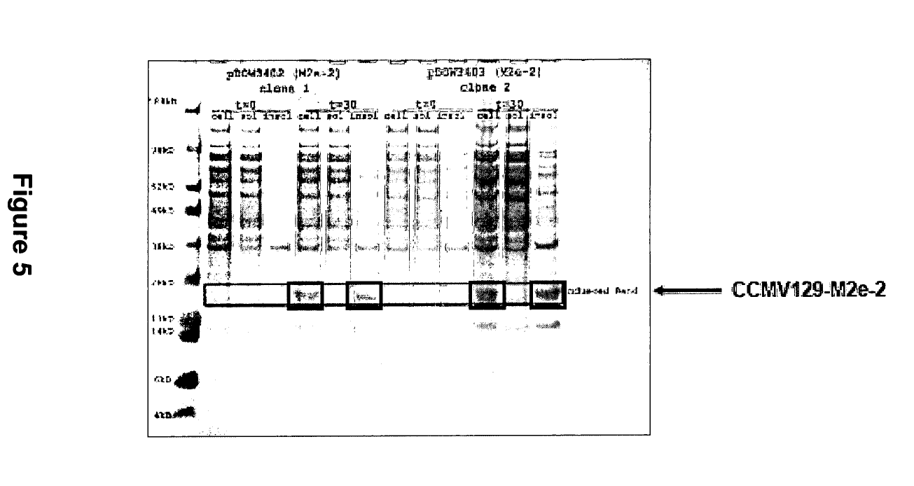

FIG. 5 shows expression of CCMV129 CP fused with M2e-2 influenza virus peptide in Pseudomonas fluorescens as detected by SDS-PAGE stained by Simply blue safe stain (Invitrogen).

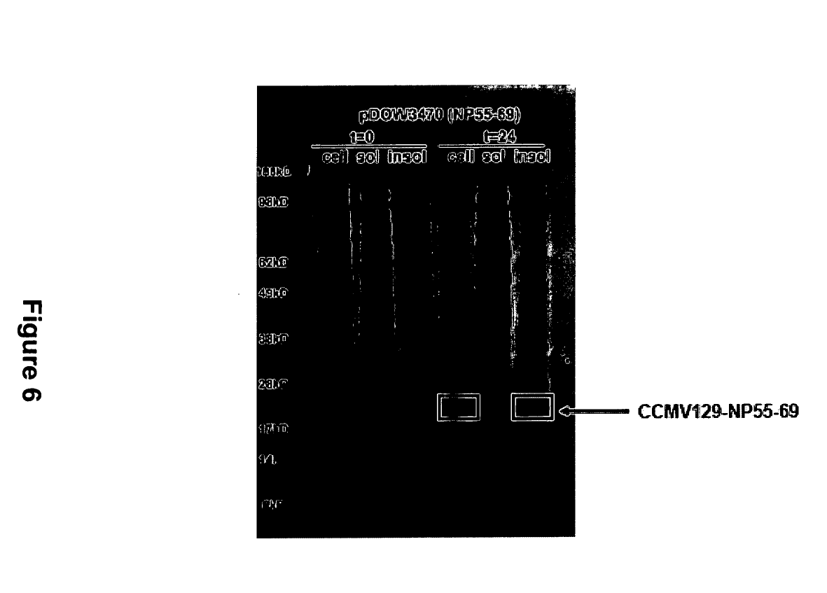

FIG. 6 shows expression of CCMV129 CP fused with NP55-69 influenza virus peptide in Pseudomonas fluorescens as detected by SDS-PAGE stained by Simply blue safe stain (Invitrogen).

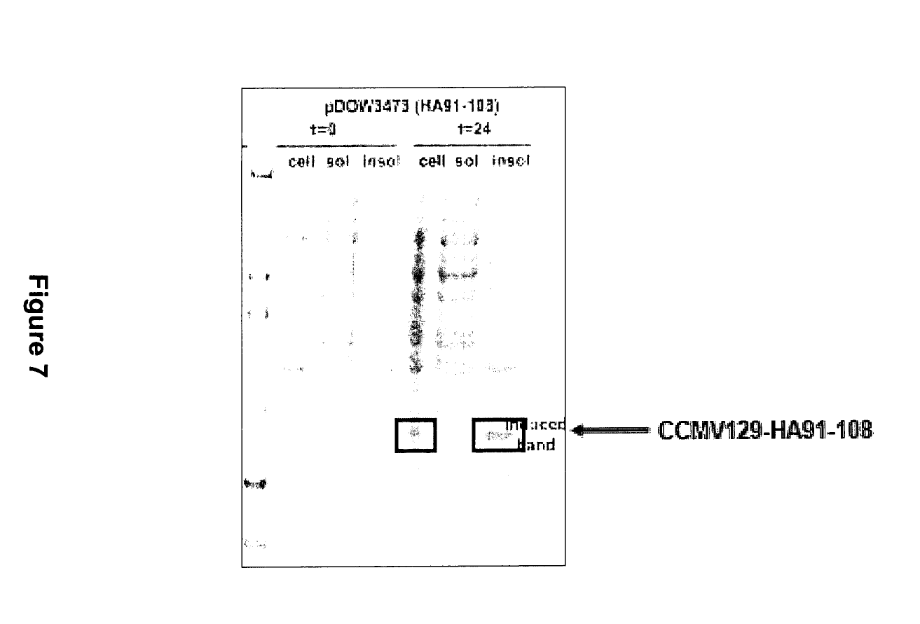

FIG. 7 shows expression of CCMV129 CP fused with NP147-158 influenza virus peptide in Pseudomonas fluorescens as detected by SDS-PAGE stained by Simply blue safe stain (Invitrogen).

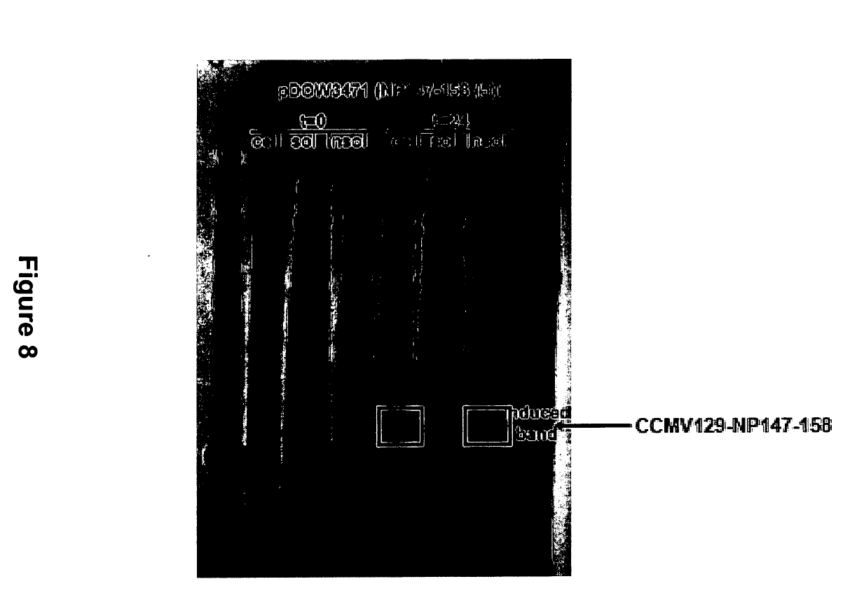

FIG. 8 shows expression of CCMV129 CP fused with HA91-108 influenza virus peptide in Pseudomonas fluorescens as detected by SDS-PAGE stained by Simply blue safe stain (Invitrogen).

### Example 6

### Purification of Recombinant CCMV VLPs

The protocol used to purify chimeric CCMV VLPs comprised the following steps: (1) Cell lysis, (2) Inclusion body (IB) wash and separation, (3) IB solubilization, (4) Heat-shock protein (HSP) contaminant removal, (5) Endotoxin removal, (6) Renaturation of coat protein, (7) Clarification, (8) VLP assembly, (9) Buffer exchange into PBS, pH 7.0, and (10) Sterile filtration.

The following buffers were used:
a. Lysis Buffer—100 mM NaCl/5 mM EDTA/0.1-0.2 mM PMSF/50 mM Tris, pH 7.5
b. Buffer AU-Low Ionic Strength—8M urea/1 mM DTT/20 mM Tris, pH 7.5
c. Buffer B w/8M urea—1M NaCl/8M urea/1 mM DTT/20 mM Tris, pH 7.5
d. CIP solution—0.5N NaOH/2M NaCl
e. Column preparation solution—100 mM Tris, pH 7.5
f. Storage solution—20% EtOH
g. Buffer B—1M NaCl/1 mM DTT/20 mM Tris, pH 7.5
h. Mustang E (Pall, cat. # MSTG25E3)-Filtered Virus Assembly Buffer—0.1 NaOAc, pH 4.8, 0.1 M NaCl, 0.0002 M PMSF
i. Mustang E-Filtered PBS pH 7.0

15-20 g of P. fluorescens wet cell paste was measured into a 50 ml conical tube and the Lysis Buffer was added to a total volume of 40 ml. Cell paste solution was vortexed and stirred until somewhat homogenous. Cells were lysed with two passes over a French Press at 1280 psi using high gear. The lysate (˜33 ml) was spun at 10000×G for 10 minutes at 4 C. The supernatant was discarded. The pellet was tight and of a powdery consistency, light in color and distinct from the cell paste. 4-5 ml of the Lysis Buffer was added to the pellet and the solution was vortexed and stirred with a spatula until the pellet has dissolved. The Lysis Buffer was added to a total volume of 40 ml. The sample was vortexed until the pellet was dissolved. The sample was spun at 10000×G for 10 minutes at 4 C. The IB wash was repeated at least one more time with the Lysis Buffer and one final time using DI water. IBs were dissolved in 4-5 ml of 8M urea/1 mM DTT/20 mM Tris, pH 7.5 by vortexing. The volume of IB solution was adjusted to 40 ml with 8M urea/1 mM DTT/20 mM Tris, pH 7.5. The solution was sonicated for 15 minutes in a chilled bath sonicator if needed and rocked overnight at 4 C followed by clarification (by spinning for 10 minutes at 10000×G at 4 C or by filtration through 0.45 um Whatman GD/X, cat. # 6976-2504). The Q-Sepharose Fast Flow (GE Healthcare) column was equilibrated using 10 Column Volume (CV) Buffer AU-Low Ionic Strength −8M urea/1 mM DTT/20 mM Tris, pH 7.5 (AU-Low). 8 ml of IB solution was loaded per ml resin and 2 ml flow-through (FT) fractions were collected. The column was washed with 6 CV using Buffer AU-Low and eluted with 5 CV of Buffer B with 8M urea. The column was cleaned and regenerated by using 6 CV CIP solution and stored in 20% ETOH. CCMV coat protein with HSP contaminant removed was found in the FT fractions that were pooled. Sartobind Q15X or Q100X filter (Sartorius) membrane was equilibrated with 10 ml Buffer AU-Low. IB solution was filtered through the filter and the filtrate was clarified. The filtered solution was added to the vessel with 5× volume of Buffer B and mixed immediately. The diluted solution was allowed to mix at 4 C for several minutes and then dialyzed against Buffer B using a 3,500 Da membrane at 4 C overnight while stirring slowly. The buffer was changed at least once. After dialysis the solution was clarified if necessary. The renatured protein solution was dialyzed into Virus Assembly Buffer for 12 hours and clarified by centrifugation or 0.2 μm filtration. The re-assembled VLP solution was concentrated in Virus Assembly Buffer over a 300 kDa membrane and exchanged into PBS, pH 7.0 using 3 buffer exchanges. The final sterile filtration was through a 0.2 μm filter.

### Example 7

### Analysis of Purified Recombinant CCMV VLPs

The purified VLPs were electrophoresed on NuPAGE 4-12% Bis-Tris gels (from Invitrogen, Cat. NP0323), having 1.0 mm×15 wells, according to manufacturer's specification. 5 ul of the sample was combined with 5 ul of 2× reducing SDS-PAGE loading buffer, and boiled for 5 minutes prior to running on the gel. The gels were stained with SimplyBlue Safe Stain, (from Invitrogen, Cat. LC6060) and destained overnight with water. Western blot detection employed anti-CCMV IgG (Accession No. AS0011 from DSMZ, Germany, the anti-Influenza A M2 protein (mouse monoclonal IgG1 kappa, cat #: MA1-082) from ABR (Affinity BioReagents) as primary antibodies, and the WESTERN BREEZE kit (from Invitrogen, Cat. WB7105), following manufacturer's protocols.

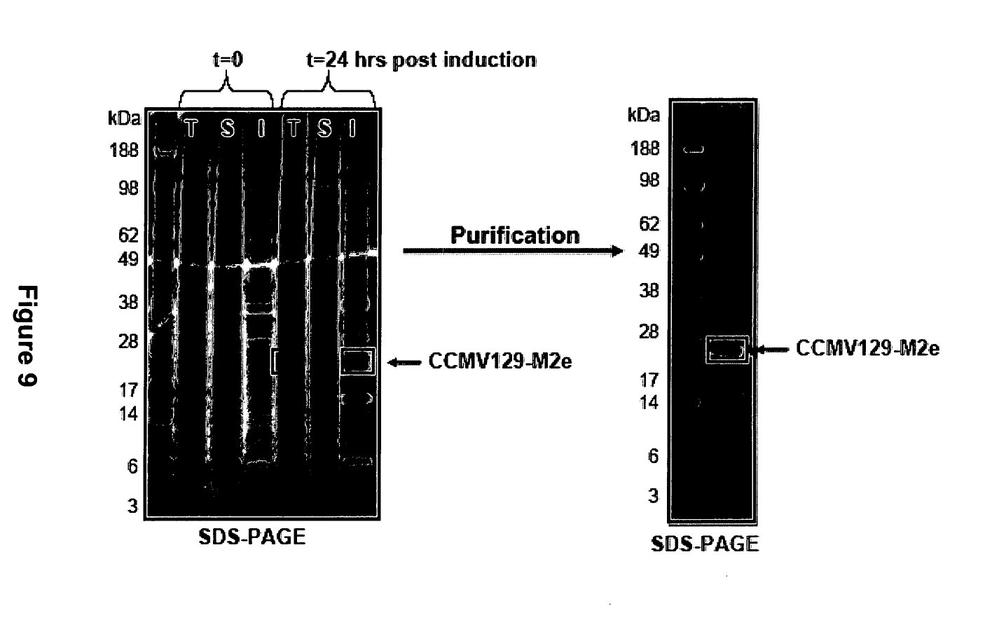

FIG. 9 shows expression and detection of purified CCMV129 CP fused with M2e-1 influenza virus peptide in Pseudomonas fluorescens as detected by SDS-PAGE stained by Simply blue safe stain (Invitrogen).

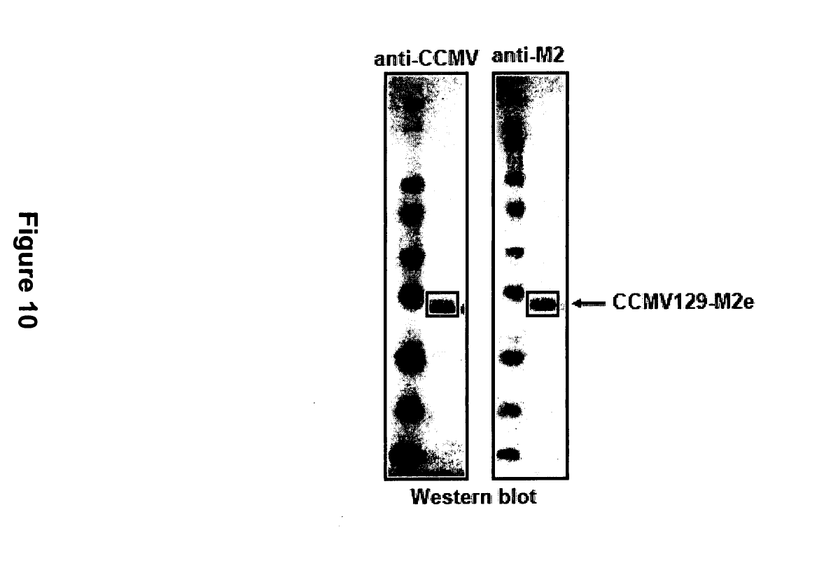

FIG. 10 shows expression of CCMV129 CP fused with M2e-1 influenza virus peptide in Pseudomonas fluorescens as detected by western blotting with anti-CCMV and anti-M2 antibodies 14B. The M2e peptide is recognized by anti-M2 antibodies.

### Example 8

### Cloning of the M2-e Universal Epitope of Influenza A Virus into Cowpea Mosaic Virus (CPMV) Coat Protein (CP)

A peptide M2e-3 derived from an M2 protein of Influenza A virus was independently cloned into CPMV small CP gene to be expressed on CPMV virus particles.

| | |
|---|---|
| M2e-3 peptide sequence: | |
| SLLTEVETPIRNEGCRCNDSSD | (Seq. ID. No. 3) |

The insert was synthesized by over-lapping DNA oligonucleotides with the thermocycling program as detailed in the Example 1.

The oligonucleotides were:

M2e-3F

(Seq. ID. No. 39)

5′ATG GAT AGC TAG CAC TCC TCC TGC TAG TCT GCT CAG
CGA AGT GGA AAC CCC GAT TCG CAA CGA AGG CTG3′
M2e-3R

(Seq. ID. No. 40)

5′TGC CTG TGA CGT CTG AAA ATG GAT CGC TGC TAT CGT
TGC AGC GGC AGC CTT CGT TGC GAA TCG G3′

Resulting PCR products were digested with AatII and NheI restriction enzymes (NEB) and subcloned into vector pDOW2604 cut with AatII, NheI and dephosphorylated. 2 μl of ligation product was transformed into Top 10 Oneshot E. coli cells (Invitrogen). Cell/ligation product mixture was incubated on ice for 30 minutes, heat-shocked for 45 seconds before addition of 0.5 ml LB animal free-soy hydrolysate (Teknova). The transformants were shaken at 37° C. for 1 hour before being plated on LB animal free-soy hydrolysate agar plate with 100 μg/ml ampicillin for selection.

The coding sequences of chimeric CPMV-CP genes (pDOW-M2e-3) were then sequenced to ensure the orientation of the inserted peptide sequence and the integrity of the modified CP gene.

### Example 9

### Production of Recombinant CPMV Containing the M2-e Universal Epitope of Influenza A Virus in Cowpea Plants

### Production of Chimeric CPMV Particles in Plants

Cowpea California #5 seeds from Ferry Morse, part number 1450, were germinated over night at room temperature in wet paper towels. Germinated seeds were transferred into soil. Seven days post germination the seedlings were inoculated with CPMV RNA1 and chimeric CPMV RNA2 in the presence of abrasive. The CPMV RNAs were produced by in vitro transcription from plasmids pDOW2605 cut with MluI and pDOW-M2e-3 cut with EcoRI. The linearized plasmid DNA was column purified by using a Qiagen clean-up column or an equivalent clean-up kit. The transcription reaction was performed by using T7 MEGAscript kit (Ambion, catalog #1334) containing CAP (40 mM) according to manufacturer instructions. Quality of transcripts was analyzed by running 1 μl of the RNA transcripts on an agarose gel.

After inoculation, the plants were grown at 25 C with a photo period of 16 hours light and 8 hours dark for two to three weeks. The leaves that showed symptoms were harvested and frozen at −80 C prior to purification.

### Purification of Chimeric CPMV Particles

40 g of CPMV infected leaf tissue was frozen at −80 C. The frozen leaf tissue was crushed by hand and poured into a Waring high speed blender, part number 8011S. 120 ml of cold AIEC binding buffer with PMSF (30 mM Tris Base pH 7.50, 0.2 mM PMSF) was poured onto the crushed leaves. The leaves were ground 2 times for 3 seconds at high speed. The solution was decanted into a 500 ml centrifuge bottle. The blender was washed with 30 ml of cold AIEC binding buffer and the wash was poured into a 500 ml centrifuge bottle. The solution was centrifuged at 15,000 G for 30 minutes to remove the plant cellular debris. The supernatant was decanted into a graduated cylinder. To precipitate the CPMV virus, cold PEG 6000 solution (20% PEG 6000, 1M NaCl) was added to the supernatant to bring the final PEG concentration to 4% PEG 6000 with 0.2M NaCl, and the solution was gently mixed. The solution was allowed to precipitate for 1 hour on ice. The virus precipitate solution was then centrifuged at 15,000 G for 30 minutes to collect the CPMV virus pellet. The supernatant was poured off and the virus pellet was immediately resuspended in anion exchange binding buffer (30 mM Tris base pH 7.50). To further purify the virus like particles, the protein mixture was fractionated by anion exchange chromatography using POROS 50 HQ strong anion exchange resin from Applied Biosystems, part number 1-2559-11. The 20 column volume gradient was from buffer A, 30 mM Tris base pH 6.75, to buffer B, 30 mM Tris base pH 6.75 with 1M NaCl. The chromatography was run with an AKTAexplorer from Amersham Biosciences, part number 18-1112-41. The first peak on the gradient, which contained the desired virus like particles, was buffer exchanged into PBS using a 100 kDa cutoff membrane Millipore spin concentrator, part number UFC910096. The samples were then stored at −80 C.

### Example 10

### Analysis of Recombinant CPMV Containing the M2-e Universal Epitope of Influenza A Virus

The stability of the small and large coat proteins were assayed with SDS PAGE. The integrity of the assembled chimeric CPMV virus particles was assayed using size exclusion chromatography. The purified particles were electrophoresed on NuPAGE 4-12% Bis-Tris gels (from Invitrogen, Cat. NP0323), having 1.0 mm×15 wells, according to manufacturer's specification. 5 ul of the sample was combined with 5 ul of 2× reducing SDS-PAGE loading buffer, and boiled for 5 minutes prior to running on the gel. The gels were stained with SimplyBlue Safe Stain, (from Invitrogen, Cat. LC6060) and destained overnight with water. Western blot detection employed polyclonal anti-CPMV polyclonal rabbit IgG J16, the anti-Influenza A M2 protein (mouse monoclonal IgG1 kappa, cat #: MA1-082) from ABR (Affinity BioReagents) as primary antibodies, and the WESTERN BREEZE kit (from Invitrogen, Cat. WB7105), following manufacturer's protocols.

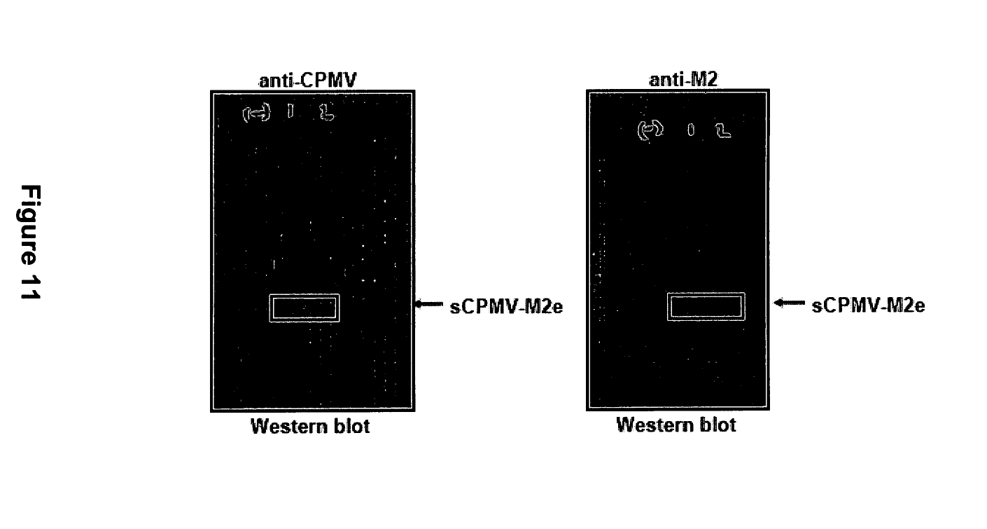

FIG. 11 shows expression of CPMV fused with M2e-1 influenza virus peptide in plants as detected by SDS-PAGE and western blotting with anti-CPMV and anti-M2 antibodies 14B. The M2e peptide is recognized by anti-M2 antibodies.

### Example 11

### HA Gene and Gene Fragments Used for Expression in Plants and Plant Cells

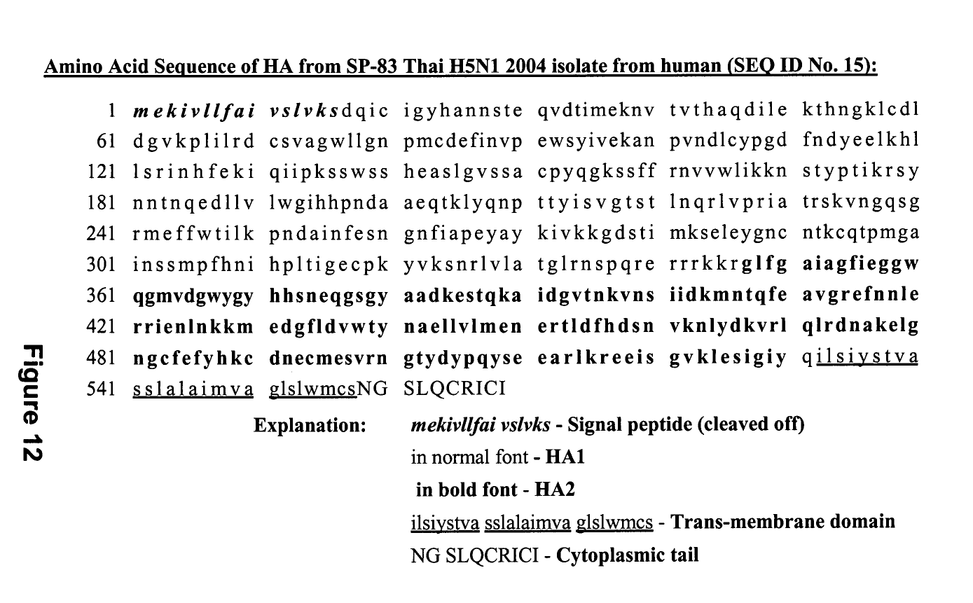

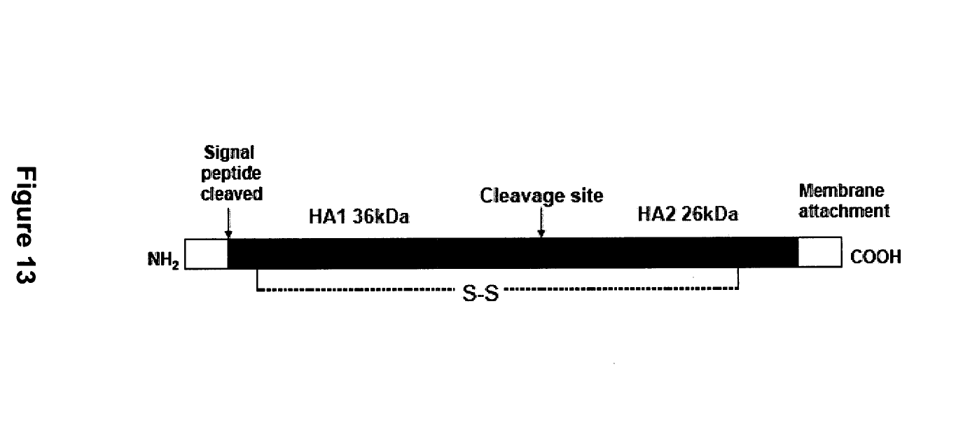

A gene encoding for the influenza HA, identified from the influenza virus A/Thailand/3(SP-83)/2004(H5N1) strain in SEQ ID No: 15 was ordered from DNA 2.0 (DNA 2.0, Menlo Park, Calif. 94025, USA) for synthesis. The HA gene synthesized was engineered to favor a plant codon usage bias and contain manufactured restriction sites flanking the gene in the absences of the same restriction sites within the gene for cloning purposes. The synthesized full length HA gene lacked the C-terminal trans-membrane domain and cytoplasmic tail. See FIG. 12 and FIG. 13. The codon optimized nucleotide sequence of the full HA gene ORF that was used for cloning and expression in plant cells is shown in Table 5, sequence SEQ ID No: 16. Amino acid sequence of the full-length HA protein translated from SEQ ID No: 16 is shown in Table 5, SEQ ID No: 17. It lacks the C-terminal trans-membrane domain and cytoplasmic tail and contains His-tag at the C-terminus of the protein. The codon optimized nucleotide sequences for HA protein fragments, HA1 and HA2, are shown in Table 5, SEQ ID No: 19 and 21. Amino acid sequence of the HA1 and HA2 protein fragments translated from SEQ ID No: 19 and 21 are shown in Table 5, SEQ ID No: 18 and 20. Both HA fragments contain the native signal peptide, have C-terminal trans-membrane domain and cytoplasmic tail removed, and contain His-tag at the C-terminus of the protein fragments.

### Example 12

### Cloning of Influenza HA Full Length, HA1, and HA2 into pDOW3451 PVX Based Expression Vector

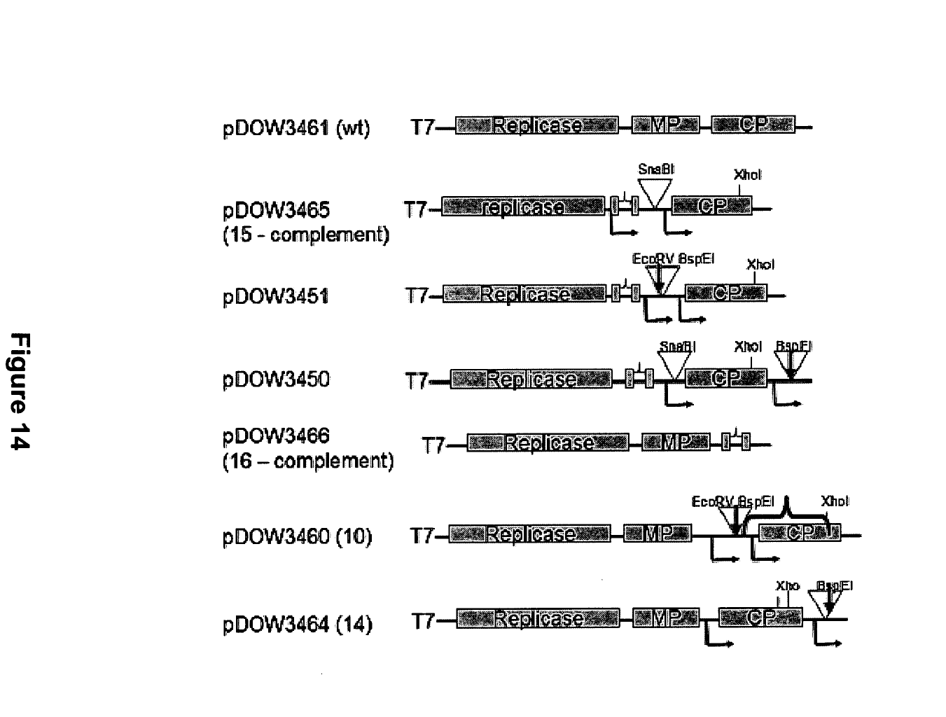

Full length HA gene was isolated using restriction enzymes EcoRV and BspE1 which allowed it to be excised from vector G01129 (DNA 2.0). Digested G01129 was run on agarose gel to separate the vector backbone from the HA gene. The HA gene was gel purified and then sub-cloned into vector pDOW3451 which was also cut with EcoRV+BspE1 and dephosphorylated using calf alkaline phosphatase (CIP). See FIG. 14. Successful cloning of the new vector pDOW3471 was verified by restriction mapping and colony PCR screening for the HA gene.

HA1 and HA2 gene fragments were isolated using G01129 as a template in a PCR reaction. The first PCR reaction served to amplify the HA1 gene which included the EcoRV restriction site, signal peptide, and the start of the ORF. The reverse primer served to add a 6×His tag on the C-terminus and the BspEI restriction site. PCR reactions were carried out using SuperPCR Mix (Invitrogen) according to the manufacturer instructions.

Primers used to amplify HA1 fragment were:

Thai 1 FHA1

(Seq. ID. No. 41)

5′GCGCGATATCAACAATGGAGAAGATAGTTC3′
Thai 3 HA1 BspE1

(Seq. ID. No. 42)

5′GCGCTCCGGATTTAGTGGTGATGGTGATGATGTCTCTTCTTACGTC3′

The second reaction served to amplify the HA2 fragment. The following primers were used:

Thai 8 FHA2 EcoRV

(Seq. ID. No. 43)

GCGATATCAACAATGGAGAAGATAGTTCTCTTGTTTGCCATCGTCAGTTT
GGTCAAATCAGGATTGTTCG3′
Thai 7 HA2 BspEI
5′GCGCTCCGGATTTAGTGGTGATGGTGATGATGTTGGTAGATACC3′

Thermocycler settings for the PCR reaction included:

1. 95° C. for 2 min

2. 94° C. for 30 sec

3. 56° C. for 30 sec

4. 68° C. for 1:10 min

5. Go to step 2 34 times

6. 68° C. for 10 min

7. 4° C.

Following PCR, products for HA1 and HA2 fragments were digested with EcoRV and BspE1, run on a DNA gel and the bands were excised for use for cloning into vector pDOW3451. Successful cloning was verified by restriction digest mapping and colony PCR screening for the HA fragments.

### Example 13

### Preparation of RNA Transcripts from pDOW3475 and pDOW3466

pDOW3471 and pDOW3466 (a helper plasmid containing the PVX genome with a deletion in the coat protein) were both linearized using restriction enzyme SpeI, Quickspin column cleaned (Qiagen) and eluted with nuclease free water (Ambion). In vitro transcription reactions were assembled as follows using components of the mMessage Machine T7 capped kit (Ambion).

| Amount | | Component |
|---|---|---|
| 10 | μL | 2X NTP/CAP |
| 2 | μL | 10X Reaction Buffer |
| 1 | μg | Linearized template DNA |
| 0.4 | μL | GTP |
| 2 | μL | Enzyme Mix |
| to 20 | μL | Nuclease-free water |

Reactions were assembled on ice, then incubated at 37° C. for 2 hours. Following in vitro RNA transcription, a small sample of each reaction was run to visualize for the RNA products.

### Example 14

### Inoculation of Nicotiana benthamiana Plants and Production of HA Protein in Plants

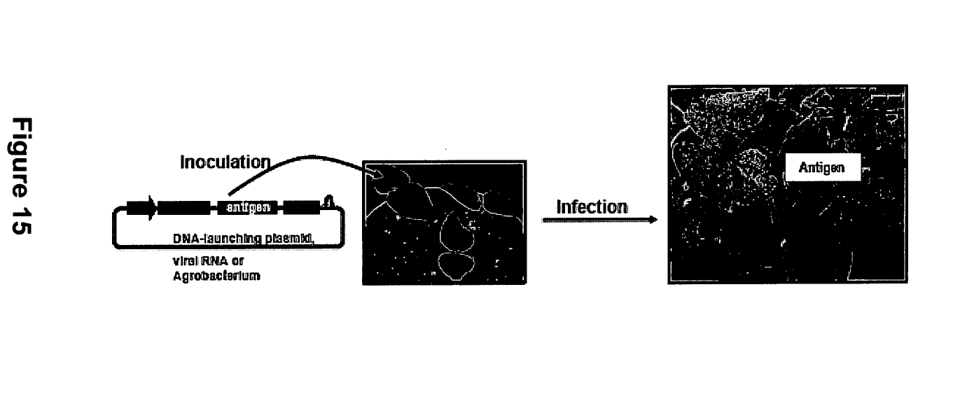

Nicotiana benthamiana plants were inoculated using in vitro transcribed RNA from pDOW3475 and pDOW3466. A single leaf from 2-3 week old plant was dusted with small amount carborundum. RNA inoculum was applied to the young leaf and on the carborundum. Using clean gloves the RNA was rubbed into the leaf tissue. One inoculum (20 μL of in vitro transcribed RNA) of pDOW3475 combined with pDOW3466 was used per plant inoculation. Plants were observed for symptom formation. See FIG. 15.

### Example 15

### Inoculation of NTI-Tobacco Cells and Production of HA Protein in Plant Cells

Transfection of Tobacco NT1 cells was performed via electroporation of in vitro transcribed RNA into NT1 protoplasts. NT1 protoplasts were prepared for electroporation by the removal of the cell wall using cellulysin and macerase. Five minutes prior to the electroporation of the pDOW3475 RNA into cells, 5 ug of a plasmid containing the HcPro gene was incubated with cells. HcPro has been previously demonstrated to prevent gene silencing hence increasing the amount of viral replication and activity. Complementation was reasoned not to be necessary for plant cell cultures to propagate the viral RNA expressing the HA gene. pDOW3466 derived RNA was used as inoculum. Immediately before electroporating, 5 μL of in vitro transcribed RNA was added to the 1 mL of processed plant cells in an ice chilled 0.4 cm gap cuvette (Biorad), and mixed quickly. Cells were pulsed at 500 μF and 250 V at a time constant of 11-13 seconds. Cells were plated onto 5 mL of NT1 plating media in a petri dish, sealed with parafilm and then allowed to grow for 48 hours at room temp.

Cells were assayed for successful transfection and production of HA. Whole cell cultures were pelleted, frozen, crushed with a pestle, and lysed in order to purify the his-tagged HA protein under native and denaturing conditions through a Ni-NTA spin column (Qiagen). Samples were then detected via a western blot utilizing primary anti-His antibodies (Qiagen 3 pack set), and secondary anti-mouse AP (Western Breeze, Invitrogen).

### Example 16

### Expression of HA or HA Fragments in Pseudomonas fluorescens

A gene encoding for the influenza HA, identified from the influenza virus A/Vietnam/2004(H5N1) strain in SEQ ID No: 25 was ordered from DNA 2.0 (DNA 2.0, Menlo Park, Calif. 94025, USA) for synthesis. The HA gene synthesized was engineered to favor a P. fluorescens codon usage bias, and contain a ribosome binding site and manufactured restriction sites flanking the gene in the absences of the same restriction sites within the gene for cloning purposes. The codon optimized nucleotide sequence of the full HA gene ORF that was used for cloning and expression in P. fluorescens cells is shown in Table 5, sequence SEQ ID No: 26. The HA protein gene was excised out of the plasmid at SpeI and XhoI and subcloned into Pseudomonas fluorescens expression plasmid pDOW1803 at SpeI and XhoI in place of buibui gene.

The resulting plasmids were transformed by electroporation into electro-competent P. fluorescens MB214. Host cells were thawed gradually in vials maintained on ice. For each transformation, 1 μL purified expression plasmid DNA was added to the host cells and the resulting mixture was swirled gently with a pipette tip to mix, and then incubated on ice for 30 min. The mixture was transferred to electroporation disposable cuvettes (BioRad Gene Pulser Cuvette, 0.2 cm electrode gap, cat no. 165-2086). The cuvettes were placed into a Biorad Gene Pulser pre-set at 200 Ohms, 25 μfarads, 2.25 kV. Cells were pulse cells briefly (about 1-2 sec). Cold LB medium was then immediately added and the resulting suspension was incubated at 30° C. for 2 hours. Cells were then plated on LB tet15 (15 ug/ml tetracycline-supplemented LB medium) agar and grown at 30° C. overnight.

One colony was picked from each plate and the picked sample was inoculated into 50 mL LB seed culture in a baffled shake flask. Liquid suspension cultures were grown overnight at 30° C. with 250 rpm shaking. 10 mL of each resulting seed culture was then used to inoculate 200 mL of shake-flask medium (i.e. yeast extracts and salt with trace elements, sodium citrate, and glycerol, pH 6.8) in a 1 liter baffled shake flask. Tetracycline was added for selection. Inoculated cultures were grown overnight at 30° C. with 250 rpm shaking and induced with IPTG for expression of the HA protein.

### Example 17

### Cloning and Expression of pbp-HA in the Periplasm of P. fluorescens DC454

Cloning:

A 24 residue phosphate binding protein secretion (pbp) signal was fused to the N-terminus of the modified influenza virus A/Vietnam/2004(H5N1) strain in SEQ ID No: 29 without its native secretion signal and C-terminal transmembrane domain.

The pbp signal was amplified out of pDOW1113 with the following primer pair:

pbpF-SpeI

(Seq. ID. No. 45)

5′-GGACTAGTAGGAGGTAACTTATGAAACTGAAACGTTTGATG-3′
pbp-HA-Rev

(Seq. ID. No. 46)

5′-GTGATAGCCGATGCAAATCTGGTCGGCCACCGCGTTGGC-3′

The modified HA protein was amplified from the shuttle plasmid containing the HA gene in SEQ ID No: 26 by PCR with the following primer pair:

pbp-HA-For

(Seq. ID. No. 47)

5′-GCCAACGCGGTGGCCGACCAGATTTGCATCGGCTATCAC-3′
HA-XhoI-Rev

(Seq. ID. No. 48)

5′-CCGCTCGAGTCATTACTGATAGATCCCGATGCTCTCC-3′

The fusion pbp-HA gene was then amplified out using the primer pairs below:

pbpF-SpeI

(Seq. ID. No. 49)

5′-GGACTAGTAGGAGGTAACTTATGAAACTGAAACGTTTGATG-3′
HA-XhoI-Rev

(Seq. ID. No. 48)

5′-CCGCTCGAGTCATTACTGATAGATCCCGATGCTCTCC-3′

PCR PROTOCOL
Reaction Mix (100 μL total volume)

| | | |
|---|---|---|
| 10 | μL | 10X PT HIFI buffer * |
| 4 | μL | 50 mM MgSO₄ * |
| 2 | μL | 10 mM dNTPs * |
| 0.25 | ng | Each Primer |
| 1-5 | ng | Template DNA |
| 1 | μL | PT HIFI Taq DNA Polymerase * |

| | |
|---|---|
| Remainder | Distilled De-ionized H₂O (ddH₂O) |

Thermocycling Steps

| | | | | |
|---|---|---|---|---|
| Step 1 | 1 Cycle | 2 | min. | 94° C. |
| Step 2 | 35 Cycles | 30 | sec. | 94° C. |
| | | 30 | sec. | 55° C. |
| | | 1 | min. | 68° C. |
| Step 3 | 1 Cycle | 10 | min. | 70° C. |

| | | | |
|---|---|---|---|
| Step 4 | 1 Cycle | Maintain | 4° C. |

Step 1: Plasmid harboring pbp signal was used as PCR template. pbpF-SpeI and pbp-HA-Rev primers were used in reaction 1. pbp-HA-For and HA-XhoI-Rev primers were used in reaction 2. PCRs were carried out according to the thermocycling protocols described above.

Step 2: PCR products 1 and 2 were used as PCR templates for this reaction. pbpF-SpeI and HA-XhoI-Rev primers were used to amplify out final PCR product.

Final PCR product was then digested by SpeI and XhoI and subcloned into P. fluorescens expression vector pDow1169 restricted with SpeI and XhoI and dephosphorylated. The ligation product was transformed by electroporation into P. fluorescens strain DC454 after purification with Micro Bio-spin 6 Chromatography columns (Biorad). The tranformants were plated on M9 Glucose plate (Teknova) after two hours shaking in LB media at 30° C. The plates were incubated at 30° C. for 48 hours. The presence of the insert was confirmed by restriction digest and sequencing.

Protein Expression:

Single transformants were inoculated into 50 ml M9 Glucose media and grown overnight. P. fluorescens cultures of 3.0-5.0 OD600 were used to inoculate shake flask cultures. Shake flasks were incubated at 30° C. with 300 rpm shaking overnight. Overnight cultures of 15.0-20.0 OD600 were induced with 300 μM isopropyl-β-D-thiogalactopyranoside (IPTG). Cultures were harvested at 24 hours post induction.

### Example 18

### Conjugation of HA or HA Fragments to CCMV Virus or Virus Like Particles In Vitro

The chimeric CCMV VLP particles containing the influenza insert are produced as described in Examples 1-7 and then further processed to conjugate the HA protein, or fragments of the HA protein, or mutant of the HA protein, or mutants of HA fragments derived as described in Examples 11-17 to the CCMV coat protein. The HA protein or protein fragments are attached to the surface exposed cysteine residues on CCMV particle or its mutants. This is achieved by oxidative coupling of cysteine thiols on CCMV to free thiol groups on the protein in the presence of 1 mM CuSO4 in 50 mM sodium acetate pH 4.8, with a molar ratio of 93 pM CCMV coat protein to 385 pM HA. The reaction is incubated for 1-4 hours. Alternatively, the HA protein is attached to the CCMV VLP surface via a method as described in Gillitzer, et al., Chemical modification of a viral cage for multivalent presentation, Chem. Commun., 2002, 2390-2391 and Chatterji et al., Chemical conjugation of heterologous proteins on the surface of Cowpea Mosaic Virus. Bioconjugate Chem., 2004, Vol. 15, 807-813.

Conjugated particles are separated from non-conjugated particles through size exclusion chromatography using a 1 cm×30 cm Superose 6 column from GE Bioscience with a mobile phase of 0.1M NaPO4 pH 7.00. Alternatively, the conjugated particles are separated from the free HA proteins or protein fragments through 4% PEG 0.2M NaCl precipitation followed by resuspension in 30 mM Tris pH 7.50.

### Example 19

### Conjugation of HA or HA Fragments to CPMV Virus or Virus Like Particles In Vitro

The chimeric CPMV VLP particles containing the influenza insert are produced as described in Examples 8-10 and further processed to conjugate the HA protein, or fragments of the HA protein, or mutants of the HA protein, or mutants of HA fragments produced as described in Examples 11-17 to the CPMV coat protein.

The HA protein is attached to the surface exposed cysteine residues on CPMV or its mutants. This is achieved by oxidative coupling of cysteine thiols on CPMV to free thiol groups on the protein in the presence of 1 mM CuSO4 in 50 mM sodium acetate pH 4.8, with a molar ratio of 93 μM CPMV coat protein to 385 μM HA. The reaction is incubated for 1-4 hours. Alternatively, the HA protein is attached to the CPMV VLP surface via a method as described in Gillitzer, et al., Chemical modification of a viral cage for multivalent presentation, Chem. Commun., 2002, 2390-2391 and Chatterji et al., Chemical conjugation of heterologous proteins on the surface of Cowpea Mosaic Virus. Bioconjugate Chem., 2004, Vol. 15, 807-813.

Conjugated particles are separated from non-conjugated particles through size exclusion chromatography using a 1 cm×30 cm Superose 6 column from GE Bioscience with a mobile phase of 0.1M NaPO4 pH 7.00. Alternatively, conjugate particles are separated from the non-conjugated HA proteins or protein fragments through 4% PEG 0.2M NaCl precipitation followed by resuspension in 30 mM Tris pH 7.50.

### Example 20

### Immunization of Mice with Chimeric CCMV and CPMV Particles Containing M2e Epitope

CCMV VLPs containing an influenza peptide insert and conjugated to an HA protein is produced as described in Example 18 and administered to Female Balb/c mice. 7 week old Balb/c mice are injected intraperitoneally with 100 μg purified of the HA conjugated CCMV VLP once every three weeks.

For intranasal immunization, 100 μg of the HA conjugated CCMV VLP is administered to anesthetized mice. A total volume of 100 μl is administered in two nostrils (50 μl per each nostril). Control mice are given a CCMV VLP with an unrelated peptide insert such as anthrax protective antigen (PA) at the same dosage schedule. Optionally, control mice are given PBS, pH 7.0.

Sera samples, nasal, and lung washes are obtained 1 day before the first administration and 2 weeks after each of the two subsequent administrations. The immunized mice are then challenged with 4000 PFU/mouse of a live mouse adapted influenza strain 2-3 weeks after the last immunization. The mice are then observed for survival. The samples are then processed for Ab titers to determine the immune response to the CCMV, HA, and M2e proteins by ELISA assay.

We claim:

1. A method of producing a composition for use as an influenza vaccine in a human or animal comprising:
i) providing a first nucleic acid encoding a recombinant capsid fusion peptide, the capsid fusion peptide comprising a plant virus capsid protein fused to an influenza viral peptide, and expressing the capsid fusion peptide in a host cell;
ii) assembling the capsid fusion peptide to form a virus or virus like particle;
iii) providing at least one second nucleic acid encoding at least one antigenic protein or protein fragment derived from an influenza virus strain, and expressing the antigenic protein or protein fragment in a host cell;
iv) isolating and purifying the antigenic protein or protein fragment; and
v) combining the virus or virus like particle and the antigenic protein or protein fragment to form a composition capable of administration to a human or animal.

2. The method of claim 1, wherein the isolated antigenic protein or protein fragment is conjugated to the virus or virus like particle.

3. The method of claim 1, wherein the isolated antigenic protein or protein fragment is derived from a newly emergent strain of influenza.

4. The method of claim 1, wherein the capsid fusion peptide comprises an influenza vial peptide derived from a conserved influenza viral peptide.

5. The method of claim 4, wherein the conserved influenza viral peptide is derived from an influenza M2 peptide.

6. The method of claim 5, wherein the M2 peptide is selected from the group consisting of Seq. ID. NO. 1-5 and 22-24.

7. The method of claim 6, wherein the M2 peptide is selected from the group consisting of Seq. ID. NO. 3, 22, 23, and 24.

8. The method of claim 1, wherein the plant virus capsid protein is derived from a CCMV or CPMV plant virus.

9. The method of claim 8, wherein the plant virus capsid protein is selected from the group consisting of Seq. ID. NO. 11-13.

10. The method of claim 1, wherein the virus or virus like particle does not contain proteins from the host cell plasma membrane or cell wall.

11. A composition comprising:
i) a recombinant capsid fusion peptide, the capsid fusion peptide comprising a plant virus capsid protein fused to an influenza viral peptide, wherein the capsid fusion peptide assembles to form a virus or virus like particle, and
ii) at least one isolated antigenic protein or protein fragment derived from an influenza virus.

12. The composition of claim 11, wherein the isolated antigenic protein or protein fragment is conjugated to the virus or virus like particle.

13. The composition of claim 11, wherein the isolated antigenic protein or protein fragment is derived from a newly emergent strain of influenza.

14. The composition of claim 11, wherein the capsid fusion peptide comprises a influenza viral peptide derived from a conserved influenza viral peptide.

15. The composition of claim 14, wherein the conserved influenza viral peptide is derived from an influenza M2 peptide.

16. The composition of claim 15, wherein the M2 peptide is selected from the group consisting of Seq. ID. NO. 1-5 and 22-24.

17. The composition of claim 16, wherein the M2 peptide is selected from the group consisting of Seq. ID. NO. 3, 22, 23, and 24.

18. The composition of claim 11, wherein the plant virus capsid protein is derived from a CCMV or CPMV plant virus.

19. The composition of claim 18, wherein the plant virus capsid protein is selected from the group consisting of Seq. ID. NO. 11-13.

20. The composition of claim 11, wherein the virus or virus like particle does not contain proteins from the host cell plasma membrane or cell wall.

21. The composition of claim 11, wherein the composition further comprises an immunostimulatory molecule.

22. The composition of claim 21, wherein the immunostimulatory molecule comprises a CpG sequence.

23. A peptide sequence selected from the group consisting of Seq. ID. NO. 3, 22, 23, and 24.
